(19) United States
(12) Patent Application Publication
(10) Pub. No.: US 2026/0218236 A1
(43) Pub. Date: Jul. 30, 2026

(54) COMPOSITIONS AND METHODS FOR THE TREATMENT OF METABOLIC LIVER DISORDERS

(71) Applicant: Poseida Therapeutics, Inc., San Diego, CA (US)

(72) Inventors: Jingjing JIANG, San Diego, CA (US); Julian David DOWN, San Diego, CA (US); Xianghong LI, San Diego, CA (US); Eric M. OSTERTAG, San Diego, CA (US)

(21) Appl. No.: 19/434,421

(22) Filed: Dec. 29, 2025

Related U.S. Application Data
(63) Continuation of application No. 17/909,101, filed on Mar. 4, 2021, now Pat. No. 12,540,335.
(60) Provisional application No. 62/985,047, filed on Mar. 4, 2020.
(60) Provisional application No. 63/121,488, filed on Dec. 4, 2020.

(51) Int. Cl. C12N 15/86; A61K 48/00; A61P 1/16; C12N 9/10; C12N 9/12

(52) U.S. Cl. CPC C12N 15/86; A61K 48/0058; A61P 1/16; C12N 9/1018; C12N 9/1241; C12Y 201/03003

(57) ABSTRACT

The present disclosure is directed to compositions and methods for the treatment of Metabolic Liver Disorders. The compositions and methods can comprise an adeno-associated virus (AAV) piggyBac polynucleotide comprising a transgene. The transgene may comprise ornithine transcarbamylase (OTC) or methylmalonyl-CoA mutase (MUT1).

### CROSS REFERENCE TO RELATED APPLICATIONS

This application is a continuation of U.S. patent application Ser. No. 17/909,101, filed Sep. 2, 2022, which is a U.S. National Phase application, filed under U.S.C. § 371, of International Application No. PCT/US2021/020929, filed Mar. 4, 2021, which claims benefit of U.S. Provisional Application No. 62/985,047, filed Mar. 4, 2020, and U.S. Provisional Application No. 63/121,488, filed Dec. 4, 2020, each of which is incorporated herein by reference in its entirety.

### SEQUENCE LISTING

The instant application contains a Sequence Listing, which has been submitted electronically in XML file format, and is herein incorporated by reference into the specification in its entirety. The XML file containing the Sequence Listing XML is named “000218-0125-302-SL.xml,” was created on Dec. 29, 2025 and is 329,050 bytes in size.

### BACKGROUND

Inherited metabolic disorders, also known as inborn errors of metabolism, are medical conditions caused by genetic defects most commonly inherited from both parents. Proper metabolism requires a complex set of chemical reactions that cells and organisms use to transform food and other nutrients into essential compounds and energy. These chemical reactions are also used as to breakdown and remove substances that are not needed, including substances that are toxic. The genetic defects that cause inherited metabolic disorders often result in a deficiency in the activity of a particular enzyme within one or more metabolic pathways. This deficiency can result in the accumulation of substances that are potentially toxic, as well as limit a subject's ability to synthesize essential compounds. There are hundreds of inherited metabolic disorders that have been characterized, including those that primarily affect the liver. Inherited metabolic disorders of the liver include urea cycle disorders (UCDs) and methylmalonic acidemia (MMA).

Urea cycle disorders (UCDs) result from genetic mutations which result in defects in the metabolism of nitrogen produced by the breakdown of proteins as well as other nitrogen-containing molecules. UCDs are commonly caused by the severe deficiency or total absence of activity of any of the first four enzymes in the urea cycle, namely Carbamoylphosphate Synthetase I (CPSI), Ornithine Transcarbamylase (OTC), Argininosuccinate Synthetase Deficiency (ASS) and Argininosuccinate Lyase Deficiency (ASL), or the cofactor producer N-Acetylglutamate Synthetase (NAGS), leading to accumulation of ammonia and other precursor metabolites. UCDs are usually diagnosed in neonates, but late-onset UCDs have been reported. UCDs can result in brain damage, cognitive defects and even death. In fact, it is hypothesized that up to 20% of sudden infant death syndrome (SIDS) cases may be attributed to a hereditary metabolic disorder such as a UCD.

Current treatments of UCDs are focused on the acute control of hyperammonemia—a common symptom of UCDs. Hyperammonemia is highly neurotoxic, requiring intensive care intervention including veno-venous hemofiltration. Long-term treatment of UCDs currently relies on alternative pathway therapy, stringent dietary protein restriction, supplementation with urea cycle intermediates and the strict avoidance of catabolismpatients with UCDs often require liver transplantation. However, the prevention of recurring hyperammonemia prior to liver transplantation can be difficult. Thus, there is a need in the art for improved compositions and methods for the treatment of UCDs.

Other metabolic disorders affecting the liver include the autosomal recessive disorder methylmalonic acidemia (MMA) (also called methylmalonic aciduria). MMA disrupts normal amino acid metabolism. The inherited forms of methylmalonic acidemia cause defects in the metabolic pathway that regulate conversion of methylmalonyl-coenzyme A (CoA) into succinyl-CoA by the enzyme methylmalonyl-CoA mutase. The result of this condition is the inability to properly digest specific fats and proteins, which in turn leads to a buildup of a toxic level of methylmalonic acid in the blood. Isolated methylmalonic acidemia is caused by changes in one of five genes: MMUT, MMAA, MMAB, MMADHC, or MCEE. Methylmalonic acidemia with homocystinuria is caused by mutations in the MMADHC, LMBRD1 and ABCD4 genes.

There is no specific treatment for methylmalonic acidemia. Treatment is currently limited to managing the symptoms and include aggressive treatment of decompensation events, special protein managed diet, vitamin B12 supplementation for the vitamin B12 responsive subtypes, medications such as carnitine, and avoidance of stressors (such as fasting or illness) that can lead to a decompensation event. Liver or kidney transplantation (or both) have been shown to help some patients. These transplants provide the body with new cells that help breakdown methylmalonic acid normally.

Previous attempts at developing gene therapies for the treatment of inherited metabolic disorders, including inherited metabolic disorders of the liver, have suffered from the inability to produce long-term expression of the delivered transgene in the target tissues. This problem is particularly pronounced in rapidly dividing tissues, such as the juvenile liver. Existing gene therapy vectors, such as AAV vectors, suffer from a lack of integration into the host's genome, resulting in only short-term expression of the delivered transgene. The compositions and methods of the present disclosure provide a solution to this long felt-need in the art by providing transposon/transposase-based AAV vectors that yield long-term expression of the delivered transgene in targeted tissues.

### SUMMARY

The present disclosure provides an adeno-associated virus (AAV) piggyBac transposon polynucleotide comprising in the 5′ to 3′ direction: a) a first AAV ITR sequence comprising the nucleic acid sequence of SEQ ID NO: 3; b) a first piggyBac ITR sequence comprising the nucleic acid sequence of SEQ ID NO: 125; c) a first insulator sequence comprising the nucleic acid sequence of SEQ ID NO: 7; d) at least one promoter sequence comprising the nucleic acid sequence of SEQ ID NO: 126; e) at least one transgene sequence comprising the nucleic acid sequence of SEQ ID NO: 22; f) a polyA sequence comprising the nucleic acid sequence of SEQ ID NO: 97; g) a second insulator sequence comprising the nucleic acid sequence of SEQ ID NO: 8; h) a second piggyBac ITR sequence comprising the nucleic acid sequence of SEQ ID NO: 96; i) at least one DNA spacer sequence comprising the nucleic acid sequence of SEQ ID NO: 129; and j) a second AAV ITR sequence comprising the nucleic acid sequence of SEQ ID NO: 4.

The present disclosure provides an AAV piggyBac transposon polynucleotide, wherein the AAV piggyBac transposon polynucleotide comprises the nucleic acid sequence of SEQ ID NO: 138.

The present disclosure provides an AAV piggyBac transposon polynucleotide comprising in the 5′ to 3′ direction: a) a first AAV ITR sequence comprising the nucleic acid sequence of SEQ ID NO: 3; b) a first piggyBac ITR sequence comprising the nucleic acid sequence of SEQ ID NO: 125; c) a first insulator sequence comprising the nucleic acid sequence of SEQ ID NO: 7; d) at least one promoter sequence comprising the nucleic acid sequence of SEQ ID NO: 132; e) at least one transgene sequence comprising the nucleic acid sequence of SEQ ID NO: 22; f) a polyA sequence comprising the nucleic acid sequence of SEQ ID NO: 97; g) a second insulator sequence comprising the nucleic acid sequence of SEQ ID NO: 8; h) a second piggyBac ITR sequence comprising the nucleic acid sequence of SEQ ID NO: 96; i) at least one DNA spacer sequence comprising the nucleic acid sequence of SEQ ID NO: 130; and j) a second AAV ITR sequence comprising the nucleic acid sequence of SEQ ID NO: 4.

The present disclosure provides an AAV piggyBac transposon polynucleotide, wherein the AAV piggyBac transposon polynucleotide comprises the nucleic acid sequence of SEQ ID NO: 139.

The present disclosure provides an adeno-associated virus (AAV) piggyBac transposon polynucleotide comprising in the 5′ to 3′ direction: a) a first AAV ITR sequence comprising the nucleic acid sequence of SEQ ID NO: 3; b) a first piggyBac ITR sequence comprising the nucleic acid sequence of SEQ ID NO: 125; c) a first insulator sequence comprising the nucleic acid sequence of SEQ ID NO: 7; d) at least one promoter sequence comprising the nucleic acid sequence of SEQ ID NO: 13; e) at least one transgene sequence comprising the nucleic acid sequence of SEQ ID NO: 22; f) a polyA sequence comprising the nucleic acid sequence of SEQ ID NO: 97; g) a second insulator sequence comprising the nucleic acid sequence of SEQ ID NO: 8; h) a second piggyBac ITR sequence comprising the nucleic acid sequence of SEQ ID NO: 96; i) at least one DNA spacer sequence comprising the nucleic acid sequence of SEQ ID NO: 131; and j) a second AAV ITR sequence comprising the nucleic acid sequence of SEQ ID NO: 4.

The present disclosure provides an AAV piggyBac transposon polynucleotide, wherein the AAV piggyBac transposon polynucleotide comprises the nucleic acid sequence of SEQ ID NO: 140.

The present disclosure provides an AAV transposase polynucleotide comprising in the 5′ to 3′ direction: a) a first AAV ITR sequence comprising the nucleic acid sequence of SEQ ID NO: 127; b) at least one promoter sequence at least one promoter sequence comprising the nucleic acid sequence of SEQ ID NO: 126; c) at least one transposase sequence comprising the nucleic acid sequence of SEQ ID NO: 48; d) a polyA sequence comprising the nucleic acid sequences of SEQ ID NO: 136; e) at least one DNA spacer sequence comprising the nucleic acid sequences of SEQ ID NO: 137; and f) a second AAV ITR sequence comprising the nucleic acid sequences of SEQ ID NO: 4.

The present disclosure provides an AAV transposase polynucleotide, wherein the AAV transposase polynucleotide comprises the nucleic acid sequence of SEQ ID NO: 144.

The present disclosure provides a vector comprising at least one of the AAV piggyBac transposon polynucleotides of the present disclosure. In some aspects, the vector can be a viral vector. In some aspects, a viral vector can be an AAV viral vector. In some aspects, an AAV viral vector can be an AAV1, AAV2, AAV3, AAV4, AAV5, AAV6, AAV7, AAV8, AAV9, AAV10 or AAV11 viral vector. In some aspects, an AAV viral vector can be an AAV-KP-1 or AAV-NP59 viral vector. In some aspects, an AAV viral vector can be an AAV-KP-1 viral vector.

The present disclosure provides a composition comprising at least one vector of the present disclosure.

The present disclosure provides methods of treating at least one metabolic liver disorder (MLD) in a subject in need thereof comprising administering to the subject at least one therapeutically effective dose of the polynucleotide, vector or composition of the presented disclosure.

The present disclosure provides methods of treating at least one MLD in a subject in need thereof, the method comprising administering to the subject: a) an AAV piggyBac transposon polynucleotide of the present disclosure, or a vector or composition comprising an AAV piggyBac transposon polynucleotide of the present disclosure; and b) an AAV transposase polynucleotide of the present disclosure, or a vector or composition comprising an AAV transposase polynucleotide of the present disclosure.

The present disclosure provides uses of the polynucleotide, vector or composition of the present disclosure for the treatment of at least one MLD in a subject in need thereof, wherein the polynucleotide, vector or composition is for administration to the subject in at least one therapeutically effective amount.

The present disclosure provides the combination of: a) an AAV piggyBac transposon polynucleotide of the present disclosure, or a vector or composition comprising an AAV piggyBac transposon polynucleotide of the present disclosure; and b) an AAV transposase polynucleotide of the present disclosure, or a vector or composition comprising an AAV transposase polynucleotide of the present disclosure for use in the treatment of at least one MLD in a subject in need thereof.

In some aspects, an at least one MLD is N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome), methylmalonic acidemia (MMA), progressive familial intrahepatic cholestasis type 1 (PFIC1), progressive familial intrahepatic cholestasis type 1 (PFIC2), progressive familial intrahepatic cholestasis type 1 (PFIC3) or any combination thereof. In some aspects, an MLD is OTC deficiency.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is a schematic of an exemplary AAV piggyBac transposon polynucleotide of the present disclosure.

FIG. 2 is a schematic of an exemplary AAV piggyBac transposon polynucleotide of the present disclosure.

FIG. 3A is a schematic of an exemplary AAV piggyBac transposon polynucleotide of the present disclosure.

FIG. 3B is a schematic of an exemplary AAV piggyBac transposon polynucleotide of the present disclosure.

FIG. 4A is a schematic of an exemplary AAV transposase polynucleotide of the present disclosure.

FIG. 4B is a schematic of an exemplary AAV transposase polynucleotide of the present disclosure.

FIG. 5 is a schematic of an exemplary AAV piggyBac transposon polynucleotide of the present disclosure.

FIG. 6 is a schematic of an exemplary AAV piggyBac transposon polynucleotide of the present disclosure.

FIG. 7 is a schematic of an exemplary AAV piggyBac transposon polynucleotide of the present disclosure.

FIG. 8 is a schematic of an exemplary AAV piggyBac transposon polynucleotide of the present disclosure.

FIG. 9 is a schematic of an exemplary AAV piggyBac transposon polynucleotide of the present disclosure.

FIG. 10 is a graph showing BLI measured in mice treated with various viral vectors of the present disclosure.

FIG. 11 is a graph showing BLI measured in mice treated with various viral vectors of the present disclosure at various concentrations.

FIG. 12 is a graph showing BLI measured in Otc^{spf-ash} mice treated with various viral vectors of the present disclosure.

FIG. 13 are a series of graphs showing the amount of non-integrated vector copy number per diploid genome for various viral vectors of the present disclosure administered to Otc^{spf-ash} mice.

FIG. 14 are a series of graphs showing the amount of non-integrated vector copy number per diploid genome and integrated vector copy number per diploid genome for various viral vectors of the present disclosure administered to Otc^{spf-ash} mice.

FIG. 15 are a series of graphs showing the amount of human OTC mRNA and SPB mRNA relative to the levels of murine OTC mRNA in Otc^{spf-ash} mice treated with viral vectors of the present disclosure.

FIG. 16 is a graph showing the correlation between human OTC mRNA or SPB mRNA to the total vector copy number per diploid genome in Otc^{spf-ash} mice treated with viral vectors of the present disclosure.

FIG. 17 is a graph showing the probability of survival in an inducible hyperammonemic mouse model treated with viral vectors of the present disclosure.

FIG. 18 is a graph showing the concentration of ammonia in plasma obtained from an inducible hyperammonemic mouse model treated with viral vectors of the present disclosure.

FIG. 19 is a graph showing liver BLI measured in mice treated with various viral vectors of the present disclosure.

FIG. 20 is a graph showing the amount of human OTC mRNA relative to the levels of murine OTC mRNA in mice treated with viral vectors of the present disclosure.

FIG. 21 is a graph showing the amount of SBP mRNA relative to the levels of murine OTC mRNA in mice treated with viral vectors of the present disclosure.

FIG. 22 is a graph showing the amount of human OTC protein relative to the levels of murine OTC protein in mice treated with viral vectors of the present disclosure.

FIG. 23 is a graph showing BLI measured in mice treated with various viral vectors of the present disclosure.

FIG. 24 is a graph showing the amount of human OTC mRNA relative to the levels of murine OTC mRNA in mice treated with viral vectors of the present disclosure.

FIG. 25 is a graph showing the amount of SBP mRNA relative to the levels of murine OTC mRNA in mice treated with viral vectors of the present disclosure.

FIG. 26 is a graph showing the amount of human OTC protein relative to the levels of murine OTC protein in mice treated with viral vectors of the present disclosure.

FIG. 27 shows immunohistochemistry analysis of liver cells isolated from mice treated with the vectors of the present disclosure.

### DETAILED DESCRIPTION

The present disclosure provides for compositions and methods for the treatment of metabolic liver disorders, including, but not limited to, urea cycle disorders (UCDs). The compositions and methods are described in further detail herein.

### Compositions of Disclosure

Adeno-Associated Virus (AAV) piggyBac Transposon Polynucleotides

The present disclosure provides compositions comprising adeno-associated virus (AAV) piggyBac transposon polynucleotides.

In some aspects an AAV piggyBac transposon polynucleotide can comprise at least one AAV inverted terminal repeat (ITR) sequence. In some aspects, an AAV piggyBac transposon polynucleotide can comprise at least one piggyBac ITR sequence. In some aspects, an AAV piggyBac transposon polynucleotide can comprise at least one insulator sequence. In some aspects an AAV piggyBac transposon polynucleotide can comprise at least one promoter sequence. In some aspects, an AAV piggyBac transposon polynucleotide can comprise at least one transgene sequence. In some aspects, an AAV piggyBac transposon polynucleotide can comprise at least one polyA sequence. In some aspects, an AAV piggyBac transposon polynucleotide can comprise at least one self-cleaving peptide sequence. In some aspects, an AAV piggyBac transposon polynucleotide can comprise at least one DNA spacer sequence. In some aspects, an AAV piggyBac transposon polynucleotide can comprise at least one Int6F sequence. In some aspects, an AAV piggyBac transposon polynucleotide can comprise at least one Int6P1 sequence. In some aspects, an AAV piggyBac transposon polynucleotide can comprise at least one Int6R sequence. In some aspects, an AAV piggyBac transposon polynucleotide can comprise at least one JctR sequence. In some aspects, an AAV piggyBac transposon polynucleotide can comprise at least one MCS sequence.

An AAV piggyBac transposon polynucleotide can comprise a first AAV ITR sequence, a first piggyBac ITR sequence, a first insulator sequence, a second insulator sequence, a second piggyBac ITR sequence, and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, a second insulator sequence, a second piggyBac ITR sequence, and a second AAV ITR sequence.

In some aspects, an AAV piggyBac transposon polynucleotide can comprise a first AAV ITR sequence, followed by a first piggyBac ITR sequence, followed by a first insulator sequence, followed by a second insulator sequence, followed by a second piggyBac ITR sequence, and followed by a second AAV ITR sequence.

An AAV piggyBac transposon polynucleotide can comprise a first AAV ITR sequence, a first piggyBac ITR sequence, a first insulator sequence, a second insulator sequence, a second piggyBac ITR sequence, and a second AAV ITR sequence, wherein between the first insulator sequence and the second insulator sequence there is any combination of at least one promoter sequence, at least one transgene sequence, at least one self-cleaving peptide sequence, and at least one polyA sequence.

In some aspects, an AAV piggyBac transposon polynucleotide can comprise in the 5′ to 3′ direction a first AAV ITR sequence, a first piggyBac ITR sequence, a first insulator sequence, a second insulator sequence, a second piggyBac ITR sequence, and a second AAV ITR sequence, wherein between the first insulator sequence and the second insulator sequence there is any combination of at least one promoter sequence, at least one transgene sequence, at least one self-cleaving peptide sequence, and at least one polyA sequence.

In some aspects, an AAV piggyBac transposon polynucleotide can comprise a first AAV ITR sequence, followed by a first piggyBac ITR sequence, followed by a first insulator sequence, followed by a second insulator sequence, followed by a second piggyBac ITR sequence, and followed by a second AAV ITR sequence, wherein between the first insulator sequence and the second insulator sequence there is any combination of at least one promoter sequence, at least one transgene sequence, at least one self-cleaving peptide sequence, and at least one polyA sequence.

An AAV piggyBac transposon polynucleotide can comprise a first AAV ITR sequence, a first piggyBac ITR sequence, a first insulator sequence, at least one promoter sequence, at least one transgene sequence, a polyA sequence, a second insulator sequence, a second piggyBac ITR sequence, and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, at least one promoter sequence, at least one transgene sequence, a polyA sequence, a second insulator sequence, a second piggyBac ITR sequence, and a second AAV ITR sequence.

In some aspects, an AAV piggyBac transposon polynucleotide can comprise a first AAV ITR sequence, followed by a first piggyBac ITR sequence, followed by a first insulator sequence, followed by at least one promoter sequence, followed by at least one transgene sequence, followed by a polyA sequence, followed by a second insulator sequence, followed by a second piggyBac ITR sequence, and followed by a second AAV ITR sequence.

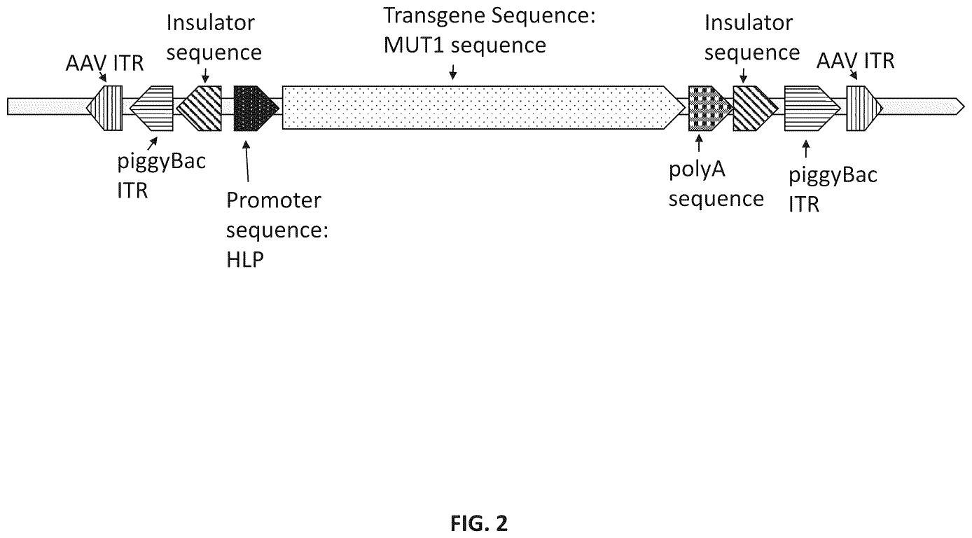

In a non-limiting example of the preceding AAV piggyBac transposon polynucleotides, the at least one promoter sequence can comprise a hybrid liver promoter (HLP) and the at least one transgene sequence can comprise a nucleic acid sequence that encodes for a methylmalonyl-CoA mutase (MUT1) polypeptide. This non-limiting example of an AAV piggyBac transposon polynucleotide is shown in FIG. 2.

An AAV piggyBac transposon polynucleotide can comprise a first AAV ITR sequence, a first piggyBac ITR sequence, a first insulator sequence, at least one promoter sequence, at least one transgene sequence, a polyA sequence, a second insulator sequence, a second piggyBac ITR sequence, at least one DNA spacer sequence and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, at least one promoter sequence, at least one transgene sequence, a polyA sequence, a second insulator sequence, a second piggyBac ITR sequence, at least one DNA spacer sequence and a second AAV ITR sequence.

In some aspects, an AAV piggyBac transposon polynucleotide can comprise a first AAV ITR sequence, followed by a first piggyBac ITR sequence, followed by a first insulator sequence, followed by at least one promoter sequence, followed by at least one transgene sequence, followed by a polyA sequence, followed by a second insulator sequence, followed by a second piggyBac ITR sequence, followed by at least one DNA spacer sequence and followed by a second AAV ITR sequence.

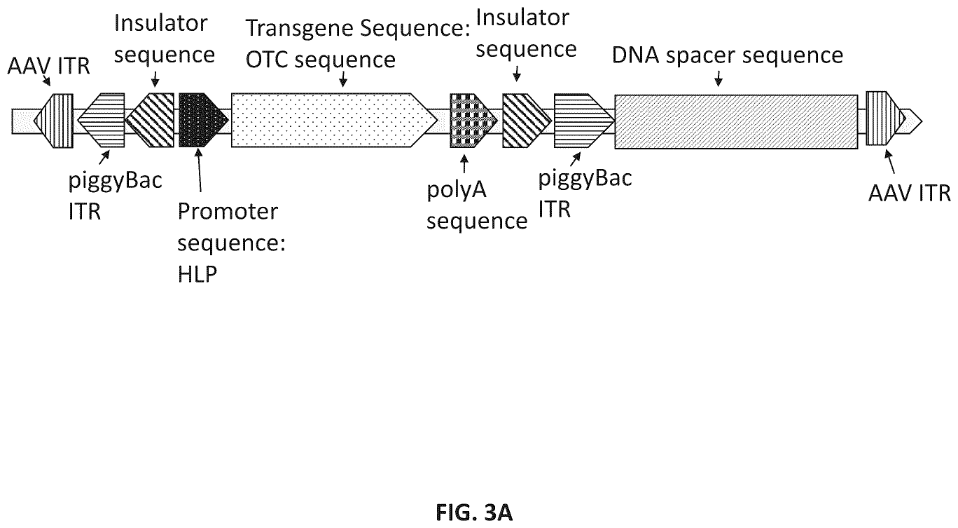

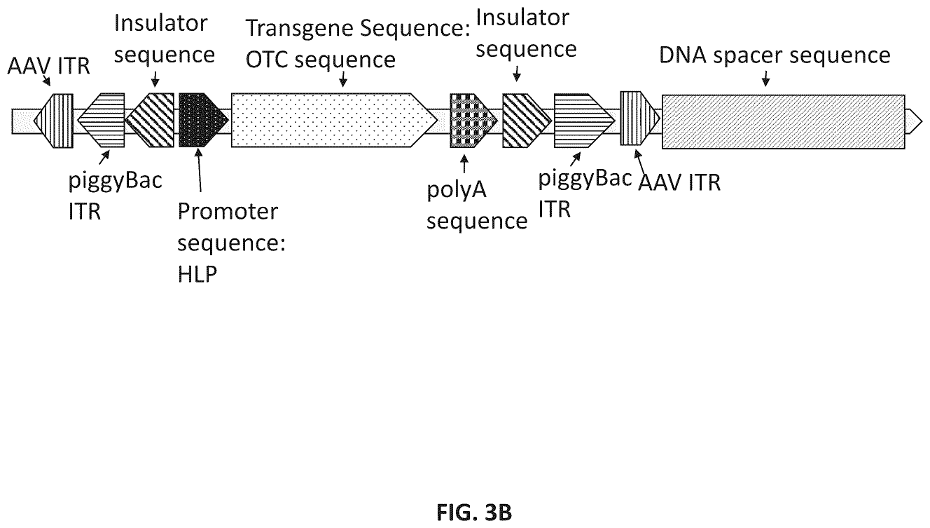

In a non-limiting example of the preceding AAV piggyBac transposon polynucleotides, the at least one promoter sequence can comprise a hybrid liver promoter (HLP) and the at least one transgene sequence can comprise a nucleic acid sequence that encodes for an ornithine transcarbamylase (OTC) polypeptide. This non-limiting example of an AAV piggyBac transposon polynucleotide is shown in FIG. 3A.

In some aspects, an AAV piggyBac transposon polynucleotide can comprise in between a second piggyBac ITR sequence and a second AAV ITR sequence, at least one DNA spacer sequence.

An AAV piggyBac transposon polynucleotide can comprise a first AAV ITR sequence, a first piggyBac ITR sequence, a first insulator sequence, at least one promoter sequence, at least one transgene sequence, a polyA sequence, a second insulator sequence, a second piggyBac ITR sequence, a second AAV ITR sequence and at least one DNA spacer sequence.

In some aspects, an AAV piggyBac transposon polynucleotide can comprise in the 5′ to 3′ direction a first AAV ITR sequence, a first piggyBac ITR sequence, a first insulator sequence, at least one promoter sequence, at least one transgene sequence, a polyA sequence, a second insulator sequence, a second piggyBac ITR sequence, a second AAV ITR sequence and at least one DNA spacer sequence.

In some aspects, an AAV piggyBac transposon polynucleotide can comprise a first AAV ITR sequence, followed by a first piggyBac ITR sequence, followed by a first insulator sequence, followed by at least one promoter sequence, followed by at least one transgene sequence, followed by a polyA sequence, followed by a second insulator sequence, followed by a second piggyBac ITR sequence, followed by a second AAV ITR sequence and followed by at least one DNA spacer sequence.

In a non-limiting example of the preceding AAV piggyBac transposon polynucleotides, the at least one promoter sequence can comprise a hybrid liver promoter (HLP) and the at least one transgene sequence can comprise a nucleic acid sequence that encodes for an ornithine transcarbamylase (OTC) polypeptide. This non-limiting example of an AAV piggyBac transposon polynucleotide is shown in FIG. 3B.

In some aspects, an AAV piggyBac transposon polynucleotide can comprise after a second AAV ITR sequence, at least one DNA spacer. A non-limiting example of an AAV piggyBac transposon polynucleotide with at least one DNA spacer following a second AAV ITR sequence is shown in FIG. 3B

An AAV piggyBac transposon polynucleotide can comprise a first AAV ITR sequence, a first piggyBac ITR sequence, a first insulator sequence, at least one promoter sequence, a first transgene sequence, at least one self-cleaving peptide sequence, an at least second transgene sequence, a polyA sequence, a second insulator sequence, a second piggyBac ITR sequence, and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, at least one promoter sequence, a first transgene sequence, at least one self-cleaving peptide sequence, an at least second transgene sequence, a polyA sequence, a second insulator sequence, a second piggyBac ITR sequence, and a second AAV ITR sequence.

In some aspects, an AAV piggyBac transposon polynucleotide can comprise a first AAV ITR sequence, followed by a first piggyBac ITR sequence, followed by a first insulator sequence, followed by at least one promoter sequence, followed by a first transgene sequence, followed by at least one self-cleaving peptide sequence, followed by an at least second transgene sequence, followed by a polyA sequence, followed by a second insulator sequence, followed by a second piggyBac ITR sequence, and followed by a second AAV ITR sequence.

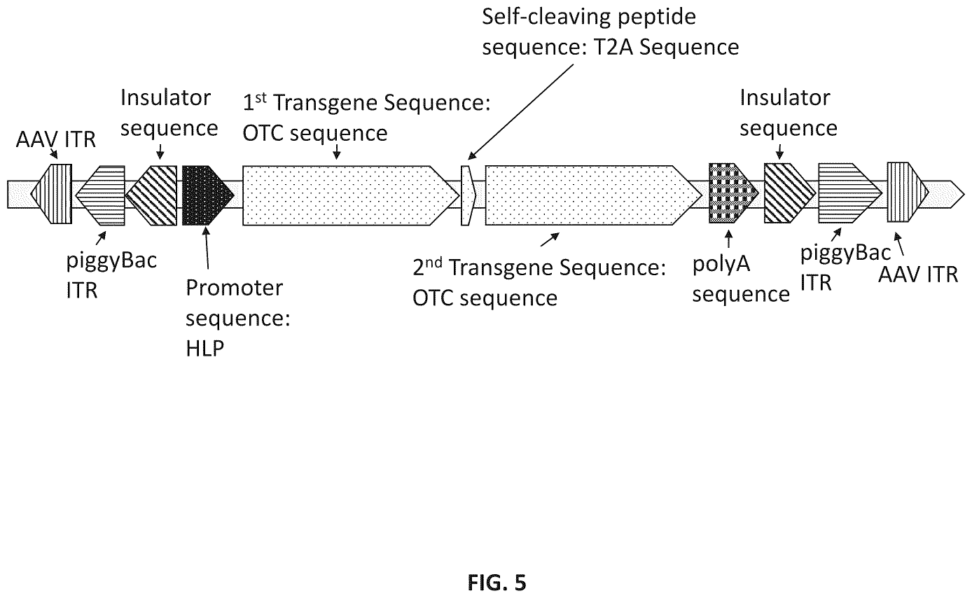

In a non-limiting example of the preceding AAV piggyBac transposon polynucleotides, the at least one promoter sequence can comprise a hybrid liver promoter (HLP), the first transgene sequence can comprise a nucleic acid sequence that encodes for an ornithine transcarbamylase (OTC) polypeptide, the at least one self-cleaving peptide sequence can comprise that encodes for a T2A peptide and the at least second transgene sequence can comprise a nucleic acid sequence that encodes for an ornithine transcarbamylase (OTC) polypeptide. This non-limiting example of an AAV piggyBac transposon polynucleotide is shown in FIG. 5.

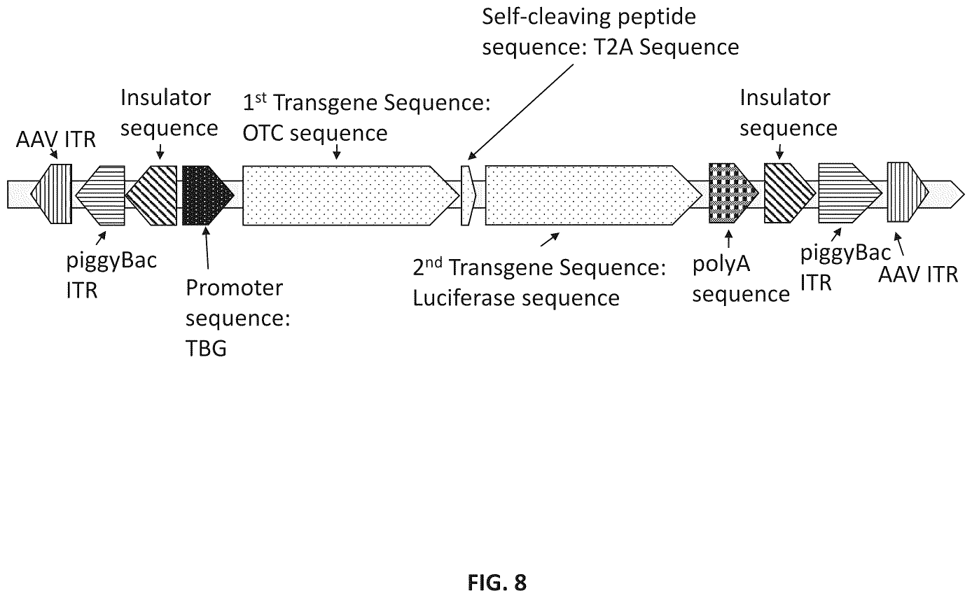

In another non-limiting example of the preceding AAV piggyBac transposon polynucleotides the at least one promoter sequence can comprise a thyroxine binding globulin (TBG) promoter, the first transgene sequence can comprise a nucleic acid sequence that encodes for an ornithine transcarbamylase (OTC) polypeptide, the at least one self-cleaving peptide sequence can comprise that encodes for a T2A peptide and the at least second transgene sequence can comprise a luciferase sequence (e.g. NanoLuc). This non-limiting example of an AAV piggyBac transposon polynucleotide is shown in FIG. 8.

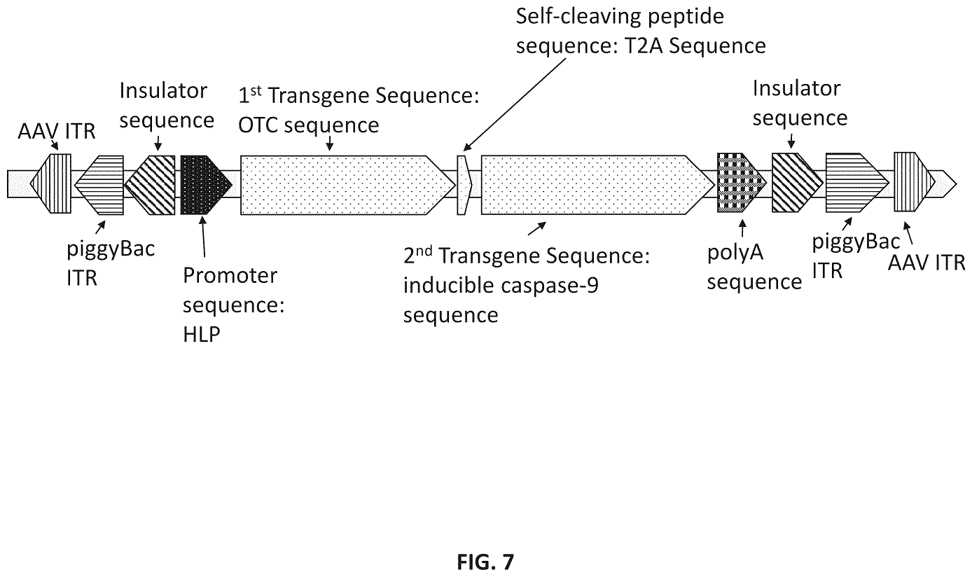

In a non-limiting of the preceding AAV piggyBac transposon polynucleotides, the at least one promoter sequence can comprise a hybrid liver promoter (HLP), the first transgene sequence can comprise a nucleic acid sequence that encodes for an ornithine transcarbamylase (OTC) polypeptide, the at least one self-cleaving peptide sequence can comprise that encodes for a T2A peptide and the at least second transgene sequence can comprise a nucleic acid sequence that encodes for an inducible caspase-9 (iCAS9) polypeptide. This non-limiting example of an AAV piggyBac transposon polynucleotide is shown in FIG. 7.

An AAV piggyBac transposon polynucleotide can comprise a first AAV ITR sequence, a first piggyBac ITR sequence, a first insulator sequence, a first promoter sequence, a first transgene sequence, at least a second promoter sequence, an at least second transgene sequence, a polyA sequence, a second insulator sequence, a second piggyBac ITR sequence, and a second AAV ITR sequence.

An AAV piggyBac transposon polynucleotide can comprise in the 5′ to 3′ direction a first AAV ITR sequence, a first piggyBac ITR sequence, a first insulator sequence, a first promoter sequence, a first transgene sequence, an at least a second promoter sequence, an at least second transgene sequence, a polyA sequence, a second insulator sequence, a second piggyBac ITR sequence, and a second AAV ITR sequence.

An AAV piggyBac transposon polynucleotide can comprise a first AAV ITR sequence, followed by a first piggyBac ITR sequence, followed by a first insulator sequence, followed by a first promoter sequence, followed by a first transgene sequence, followed by an at least a second promoter sequence, followed by an at least second transgene sequence, followed by a polyA sequence, followed by a second insulator sequence, followed by a second piggyBac ITR sequence and followed by a second AAV ITR sequence.

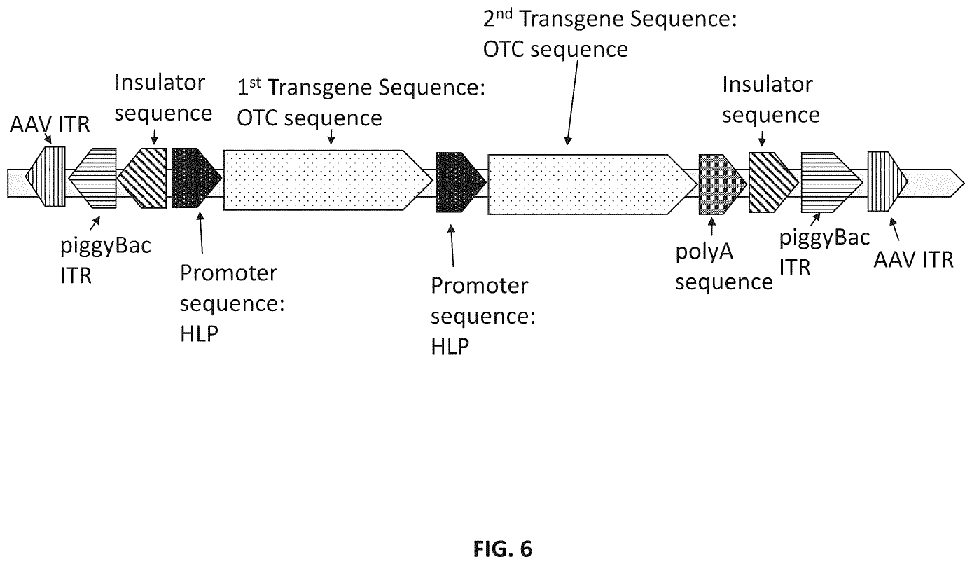

In a non-limiting example of the preceding AAV piggyBac transposon polynucleotides, the first promoter sequence can comprise a hybrid liver promoter (HLP), the first transgene sequence can comprise a nucleic acid sequence that encodes for an ornithine transcarbamylase (OTC) polypeptide, the at least second promoter sequence can comprise a HLP and the at least second transgene sequence can comprise a nucleic acid sequence that encodes for an ornithine transcarbamylase (OTC) polypeptide. This non-limiting example of an AAV piggyBac transposon polynucleotide is shown in FIG. 6.

In some aspects, an AAV piggyBac transposon polynucleotide can comprise more than one transgene sequence. In some aspects wherein the AAV piggyBac transposon polynucleotide comprises more than one transgene sequence, individual transgene sequences can be separated by a self-cleaving peptide sequence. In some aspects wherein the AAV piggyBac transposon polynucleotide comprises more than one self-cleaving peptide sequence, the self-cleaving peptide sequences can be the same or can be different.

In some aspects, an AAV piggyBac transposon polynucleotide can comprise more than one transgene sequence. In some aspects wherein the AAV piggyBac transposon polynucleotide comprises more than one transgene sequence, the AAV piggyBac transposon may comprise multiple copies of a nucleic acid sequence that encodes for the same polypeptide. In a non-limiting example, an AAV piggyBac transposon polynucleotide can comprise a first transgene sequence and a second transgene sequence, wherein the first transgene sequence and the second transgene sequence comprise a nucleic acid that encodes of an ornithine transcarbamylase (OTC) polypeptide.

In some aspects, an AAV piggyBac transposon polynucleotide can comprise more than one promoter sequence. In some aspects wherein the AAV piggyBac transposon polynucleotide comprises more than one promoter sequence, the promoter sequences can be the same or the promoter sequences can be different.

In some aspects, an AAV piggyBac transposon polynucleotide can comprise a first AAV ITR sequence, a first piggyBac ITR sequence, a first insulator sequence, at least one promoter sequence, a first transgene sequence, a first self-cleaving peptide sequence, a second transgene sequence, an at least second self-cleaving peptide sequence, at least a third transgene sequence, a polyA sequence, a second insulator sequence and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, at least one promoter sequence, a first transgene sequence, a first self-cleaving peptide sequence, a second transgene sequence, an at least second self-cleaving peptide sequence, at least a third transgene sequence, a polyA sequence, a second insulator sequence and a second AAV ITR sequence.

In some aspects, an AAV piggyBac transposon polynucleotide can comprise a first AAV ITR sequence, followed by a first piggyBac ITR sequence, followed by a first insulator sequence, followed by at least one promoter sequence, followed by a first transgene sequence, followed by a first self-cleaving peptide sequence, followed by a second transgene sequence, followed by an at least second self-cleaving peptide sequence, followed by at least a third transgene sequence, followed by a polyA sequence, followed by a second insulator sequence and followed by a second AAV ITR sequence.

In some aspects, an AAV piggyBac transposon polynucleotide can comprise a first AAV ITR sequence, a first piggyBac ITR sequence, a first insulator sequence, at least one promoter sequence, a first transgene sequence, a first self-cleaving peptide sequence, a second transgene sequence, an at least second self-cleaving peptide sequence, at least a third transgene sequence, a polyA sequence, a second insulator sequence, a second piggyBac ITR sequence and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, at least one promoter sequence, a first transgene sequence, a first self-cleaving peptide sequence, a second transgene sequence, an at least second self-cleaving peptide sequence, at least a third transgene sequence, a polyA sequence, a second insulator sequence, a second piggyBac ITR sequence and a second AAV ITR sequence.

In some aspects, an AAV piggyBac transposon polynucleotide can comprise a first AAV ITR sequence, followed by a first piggyBac ITR sequence, followed by a first insulator sequence, followed by at least one promoter sequence, followed by a first transgene sequence, followed by a first self-cleaving peptide sequence, followed by a second transgene sequence, followed by an at least second self-cleaving peptide sequence, followed by at least a third transgene sequence, followed by a polyA sequence, followed by a second insulator sequence, followed by a second piggyBac ITR sequence and followed by a second AAV ITR sequence

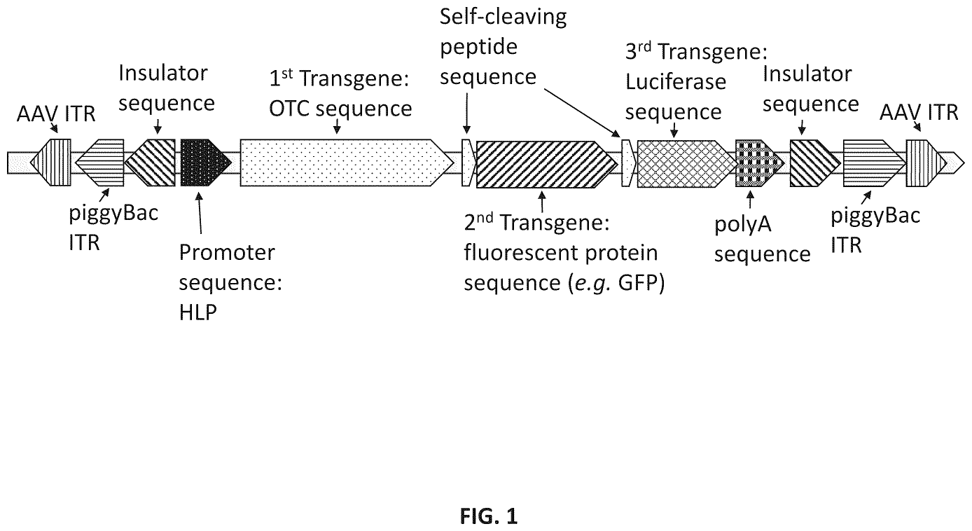

In a non-limiting example of the preceding AAV piggyBac transposon polynucleotide, the at least one promoter sequence can comprise a hybrid liver promoter (HLP), the first transgene sequence can comprise a nucleic acid sequence that encodes for an ornithine transcarbamylase (OTC) polypeptide, the second transgene sequence can comprise a fluorescent protein sequence (e.g. GFP or eGFP) and the at least third transgene sequence can comprise a luciferase sequence (e.g. NanoLuc). In this non-limiting example, both of the first self-cleaving peptide sequence and the at least second self-cleaving peptide sequence can comprise a can comprise a nucleic acid sequence that encodes for a T2A peptide or a GSG-T2A peptide. This non-limiting example of an AAV piggyBac transposon polynucleotide is shown in FIG. 1.

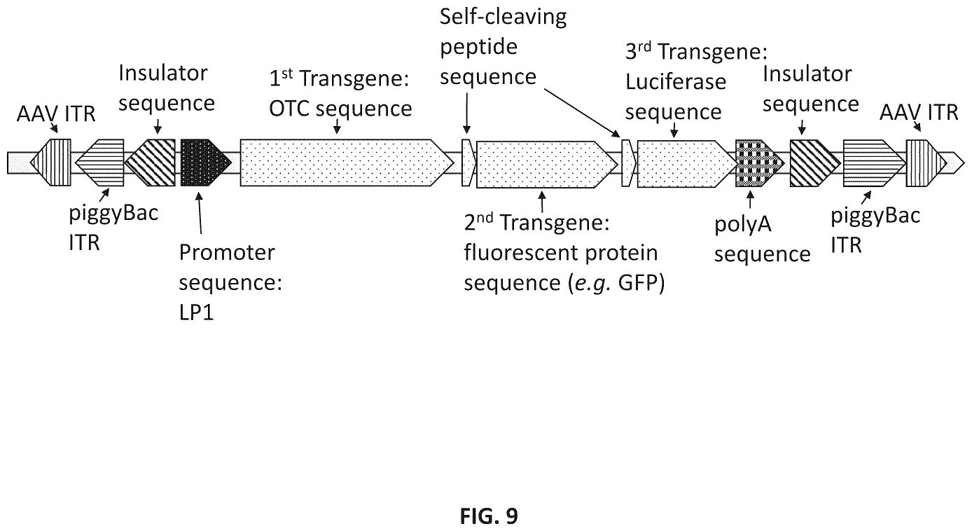

In another non-limiting example of the preceding AAV piggyBac transposon polynucleotide, the at least one promoter sequence can comprise a LP1 promoter, the first transgene sequence can comprise a nucleic acid sequence that encodes for an ornithine transcarbamylase (OTC) polypeptide, the second transgene sequence can comprise a fluorescent protein sequence (e.g. GFP or eGFP) and the at least third transgene sequence can comprise a luciferase sequence (e.g. NanoLuc). In this non-limiting example, both of the first self-cleaving peptide sequence and the at least second self-cleaving peptide sequence can comprise a can comprise a nucleic acid sequence that encodes for a T2A peptide or a GSG-T2A peptide. This non-limiting example of an AAV piggyBac transposon polynucleotide is shown in FIG. 9.

In some aspects, an AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 104.

### AAV ITR Sequences

In some aspects, an AAV ITR sequence can comprise any AAV ITR sequence known in the art. In some aspects, an AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 1-4, 93-94, 105-106 and 127.

In some aspects, a first AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 1 and a second AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 2.

In some aspects, a first AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 3 and a second AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 4.

In some aspects, a first AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 93 and a second AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 94.

In some aspects, a first AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 105 and a second AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 106.

In some aspects, a first AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 127 and a second AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 4.

### PiggyBac ITR Sequences

In some aspects, a piggyBac ITR sequence can comprise any piggyBac ITR sequence known in the art. In some aspects, a piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 5-6, 86-90, 95-96 and 125.

In some aspects, a first piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 5 and a second piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 6.

In some aspects, a first piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 6 and a second piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 5.

In some aspects, a first piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 95 and a second piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 96.

In some aspects, a first piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 125 and a second piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 96.

In some aspects of the methods of the present disclosure, a piggyBac ITR sequence, such as a first piggyBac ITR sequence and/or a second piggyBac ITR sequence in an AAV piggyBac transposon can comprise, consist essentially of, or consist of a Sleeping Beauty transposon ITR, a Helraiser transposon ITR, a Tol2 transposon ITR, a TcBuster transposon ITR or any combination thereof.

In some aspects, a piggyBac ITR sequence of the present disclosure can be flanked on either or both ends by at least one of the following sequences: 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′. In some aspects, a piggyBac ITR sequence can be flanked by 5′-TTAA-3′. Thus, any AAV transposase polynucleotide, AAV piggyBac transposon polynucleotide and/or any liver nanoplasmid of the present disclosure can further comprise any one of: 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′ flanking a piggyBac ITR sequence.

### Insulator Sequences

In some aspects, an insulator sequence can comprise any insulator sequence known in the art. In some aspects, an insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 7-8, 77-80 and 91-92.

In some aspects, a first insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 7 and a second insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any SEQ ID NO: 8.

In some aspects, a first insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 77 and a second insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any SEQ ID NO: 78.

In some aspects, a first insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 79 and a second insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any SEQ ID NO: 80.

In some aspects, a first insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 91 and a second insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any SEQ ID NO: 92.

### Promoter Sequences

In some aspects, a promoter sequence can comprise any promoter sequence known in the art. In some aspects, a promoter sequence can comprise any liver-specific promoter sequence known in the art.

In some aspects, a promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 9-16, 69, 107, 126, 132, 145 and 146.

In some aspects, a promoter sequence can comprise a hybrid liver promoter (HLP). An HLP can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 9, 107 or 126.

In some aspects, a promoter sequence can comprise an LP1 promoter. An LP1 promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 10 or 132.

In some aspects, a promoter sequence can comprise a leukocyte-specific expression of the pp52 (LSP1) long promoter. An LSP1 long promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 11.

In some aspects, a promoter sequence can comprise a thyroxine binding globulin (TBG) promoter. A TBG promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 12.

In some aspects, a promoter sequence can comprise a wTBG promoter. A wTBG promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 13.

In some aspects, a promoter sequence can comprise a hepatic combinatorial bundle (HCB) promoter. An HCB promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 14.

In some aspects, a promoter sequence can comprise a 2×ApoE-hAAT promoter. An 2×ApoE-hAAT promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 15.

In some aspects, a promoter sequence can comprise a leukocyte-specific expression of the pp52 (LSP1) plus chimeric intron promoter. An LSP1 plus chimeric intron promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 16.

In some aspects, a promoter sequence can comprise a cytomegalovirus (CMV) promoter. A CMV promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 69.

In some aspects, a promoter sequence can comprise a TTR promoter. A TTR promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 145.

In some aspects, a promoter sequence can comprise a TTRm promoter. A TTRm promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 146.

### Transgene Sequences

In some aspects, a transgene sequence can comprise a nucleic acid sequence that encodes for a methylmalonyl-CoA mutase (MUT1) polypeptide. In some aspects, a transgene sequence can comprise a nucleic acid sequence that encodes for a MUT1 polypeptide, wherein the MUT1 polypeptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 17, 18, 121 or 122. In some aspects, a nucleic acid sequence that encodes for a MUT1 polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 19, 20 or 111-120.

In some aspects, a transgene sequence can comprise a nucleic acid sequence that encodes for an ornithine transcarbamylase (OTC) polypeptide. In some aspects, a transgene sequence can comprise a nucleic acid sequence that encodes for an OTC polypeptide, wherein the OTC polypeptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 21, 81, 123 or 124. In some aspects, a nucleic acid sequence that encodes for an OTC polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 22, 23, 82 and 83.

In some aspects, a transgene sequence can comprise a nucleic acid sequence that encodes for an iCAS9 polypeptide. In some aspects, a transgene sequence can comprise a nucleic acid sequence that encodes for an iCAS9 polypeptide, wherein the iCAS9 polypeptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 24 or 84. In some aspects, a nucleic acid sequence that encodes for an iCAS9 polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 25 or 85.

In some aspects, a transgene sequence can be codon optimized according to methods known in the art.

In some aspects, the nucleic acid sequence encoding a polypeptide (e.g. OTC, MUT1, etc.) can be a codon optimized nucleic acid sequence that encodes for the polypeptide. A codon optimized nucleic acid sequence encoding a polypeptide can comprise, consist essentially of, or consist of a nucleic acid sequence that is no more than 65%, 70%, 75%, 80%, 85%, 90%, 95%, 96%, 97%, 98%, 99% (or any percentage in between) identical to the wildtype human nucleic acid sequence encoding the polypeptide.

SEQ ID NOs: 19, 20, 22, 23, 82 and 83 are unique codon optimized nucleic acid sequences that can be included in the polynucleotides, vectors and compositions of the present disclosure.

In some aspects, a codon optimized nucleic acid sequence encoding a polypeptide, such as those put forth in SEQ ID NOs: 19, 20, 22, 23, 82 and 83, can comprise no donor splice sites. In some aspects, a codon optimized nucleic acid sequence encoding a polypeptide can comprise no more than about one, or about two, or about three, or about four, or about five, or about six, or about seven, or about eight, or about nine, or about ten donor splice sites. In some aspects, a codon optimized nucleic acid sequence encoding a polypeptide comprises at least one, or at least two, or at least three, or at least four, or at least five, or at least six, or at least seven, or at least eight, or at least nine, or at least ten fewer donor splice sites as compared to the wildtype human nucleic acid sequence encoding the polypeptide. Without wishing to be bound by theory, the removal of donor splice sites in the codon optimized nucleic acid sequence can unexpectedly and unpredictably increase expression of the polypeptide in vivo, as cryptic splicing is prevented. Moreover, cryptic splicing may vary between different subjects, meaning that the expression level of the polypeptide comprising donor splice sites may unpredictably vary between different subjects.

In some aspects, a codon optimized nucleic acid sequence encoding a polypeptide, such as those put forth in SEQ ID NOs: 19, 20, 22, 23, 82 and 83, can have a GC content that differs from the GC content of the wildtype human nucleic acid sequence encoding the polypeptide. In some aspects, the GC content of a codon optimized nucleic acid sequence encoding a polypeptide is more evenly distributed across the entire nucleic acid sequence, as compared to the wildtype human nucleic acid sequence encoding the polypeptide. Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects, the codon optimized nucleic acid sequence encoding a polypeptide, such as those put forth in SEQ ID NOs: 19, 20, 22, 23, 82 and 83, exhibits at least 5%, at least 10%, at least 20%, at least 30%, at least 50%, at least 75%, at least 100%, at least 200%, at least 300%, at least 500%, or at least 1000% increased expression in a human subject relative to a wild-type or non-codon optimized nucleic acid sequence encoding the polypeptide.

In some aspects, an at least one transgene sequence can be operatively linked to at least one promoter sequence present in the same polynucleotide.

### PolyA Sequences

In some aspects, a polyA sequence can comprise any polyA sequence known in the art. Non-limiting examples of polyA sequences include, but are not limited to, SV40 polyA sequences In some aspects, polyA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 26-27, 97, 108, 128 and 136.

### Self-Cleaving Peptide Sequence

In some aspects, a self-cleaving peptide sequence can comprise any self-cleaving peptide sequence known in the art. In some aspects, a self-cleaving peptide sequence can comprise an 2A self-cleaving peptide sequence known in the art. Non-limiting examples of self-cleaving peptides include a T2A peptide, GSG-T2A peptide, an E2A peptide, a GSG-E2A peptide, an F2A peptide, a GSG-F2A peptide, a P2A peptide, or a GSG-P2A peptide.

In some aspects, a self-cleaving peptide sequence can comprise a nucleic acid sequence that encodes for a T2A peptide. In some aspects, a self-cleaving peptide sequence comprise a nucleic acid sequence that encodes for a T2A peptide, wherein the T2A peptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 28.

In some aspects, a self-cleaving peptide sequence can comprise a nucleic acid sequence that encodes for a GSG-T2A peptide. In some aspects, a self-cleaving peptide sequence comprise a nucleic acid sequence that encodes for a GSG-T2A peptide, wherein the GSG-T2A peptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 29. In some aspects, a nucleic acid sequence that encodes for a GSG-T2A peptide can comprise, consist essentially of or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 30-32 and 135.

In some aspects, a self-cleaving peptide sequence can comprise a nucleic acid sequence that encodes for an E2A peptide. In some aspects, a self-cleaving peptide sequence comprise a nucleic acid sequence that encodes for an E2A peptide, wherein the E2A peptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 33.

In some aspects, a self-cleaving peptide sequence can comprise a nucleic acid sequence that encodes for a GSG-E2A peptide. In some aspects, a self-cleaving peptide sequence comprise a nucleic acid sequence that encodes for a GSG-E2A peptide, wherein the GSG-E2A peptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 34.

In some aspects, a self-cleaving peptide sequence can comprise a nucleic acid sequence that encodes for a F2A peptide. In some aspects, a self-cleaving peptide sequence comprise a nucleic acid sequence that encodes for a F2A peptide, wherein the F2A peptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 35.

In some aspects, a self-cleaving peptide sequence can comprise a nucleic acid sequence that encodes for a GSG-F2A peptide. In some aspects, a self-cleaving peptide sequence comprise a nucleic acid sequence that encodes for a GSG-F2A peptide, wherein the GSG-F2A peptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 36.

In some aspects, a self-cleaving peptide sequence can comprise a nucleic acid sequence that encodes for a P2A peptide. In some aspects, a self-cleaving peptide sequence comprise a nucleic acid sequence that encodes for a P2A peptide, wherein the P2A peptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 37.

In some aspects, a self-cleaving peptide sequence can comprise a nucleic acid sequence that encodes for a GSG-P2A peptide. In some aspects, a self-cleaving peptide sequence comprise a nucleic acid sequence that encodes for a GSG-P2A peptide, wherein the GSG-P2A peptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 38.

### DNA Spacer Sequences

In some aspects, a DNA spacer sequence can comprise, consist essentially of or consist of a nucleic acid sequence at least 65%, 70%, 75%, 80%, 85%, 90%, 95%, 96%, 97%, 98%, 99% or 100% (or any percentage in between) identical to any one of the nucleic acid sequence put forth in SEQ ID NOs: 103, 109, 129-131 and 137.

DNA spacer sequences can be located at any position within an AAV piggyBac transposon polynucleotide or an AAV piggyBac transposase polynucleotide.

### Int6F Sequences

In some aspects, an Int6F sequence can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 98. In some aspects, an Int6F sequence can be located between a polyA sequence and a second insulator sequence.

### Int6P1 Sequences

In some aspects, an Int6P1 sequence can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 99. In some aspects, an IntP1 sequence can be located between a polyA sequence and a second insulator sequence.

### Int6R Sequences

In some aspects, an Int6R sequence can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 100. In some aspects, an Int6R sequence can be located between a polyA sequence and a second insulator sequence.

### JctR Sequences

In some aspects, a JctR sequence can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 101. In some aspects, a JctR sequence can be located between a second piggyBac ITR sequence and a second AAV ITR sequence.

In some aspects, an AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 138.

In some aspects, an AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 139.

In some aspects, an AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 140.

In some aspects, an AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 141.

In some aspects, an AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 142.

In some aspects, an AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 143.

### MCS Sequences

In some aspects, a MCS sequence can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 102. In some aspects, an MCS sequence can be located between a second piggyBac ITR sequence and a second AAV ITR sequence.

### AAV Transposase Polynucleotides

The present disclosure provides compositions comprising AAV transposase polynucleotides.

In some aspects an AAV transposase polynucleotide can comprise at least one AAV inverted terminal repeat (ITR) sequence. In some aspects an AAV transposase polynucleotide can comprise at least one promoter sequence. In some aspects, an AAV transposase polynucleotide can comprise at least one transposase sequence. In some aspects, an AAV transposon polynucleotide can comprise at least one polyA sequence. In some aspects, an AAV transposon polynucleotide can comprise at least one DNA spacer sequence.

In some aspects, an AAV transposase polynucleotide can comprise a first AAV ITR sequence, at least one promoter sequence, at least one transposase sequence, a polyA sequence and a second AAV ITR sequence.

In some aspects, an AAV transposase polynucleotide can comprise in the 5′ to 3′ direction a first AAV ITR sequence, at least one promoter sequence, at least one transposase sequence, a polyA sequence and a second AAV ITR sequence.

In some aspects, an AAV transposase polynucleotide can comprise a first AAV ITR sequence, followed by at least one promoter sequence, followed by at least one transposase sequence, followed by a polyA sequence and followed by a second AAV ITR sequence.

In some aspects, an AAV transposase polynucleotide can comprise a first AAV ITR sequence, at least one promoter sequence, at least one transposase sequence, a polyA sequence, at least one DNA spacer sequence and a second AAV ITR sequence.

In some aspects, an AAV transposase polynucleotide can comprise in the 5′ to 3′ direction a first AAV ITR sequence, at least one promoter sequence, at least one transposase sequence, a polyA sequence, at least one DNA spacer sequence and a second AAV ITR sequence.

In some aspects, an AAV transposase polynucleotide can comprise a first AAV ITR sequence, followed by at least one promoter sequence, followed by at least one transposase sequence, followed by a polyA sequence, followed by at least one DNA spacer sequence and followed by a second AAV ITR sequence.

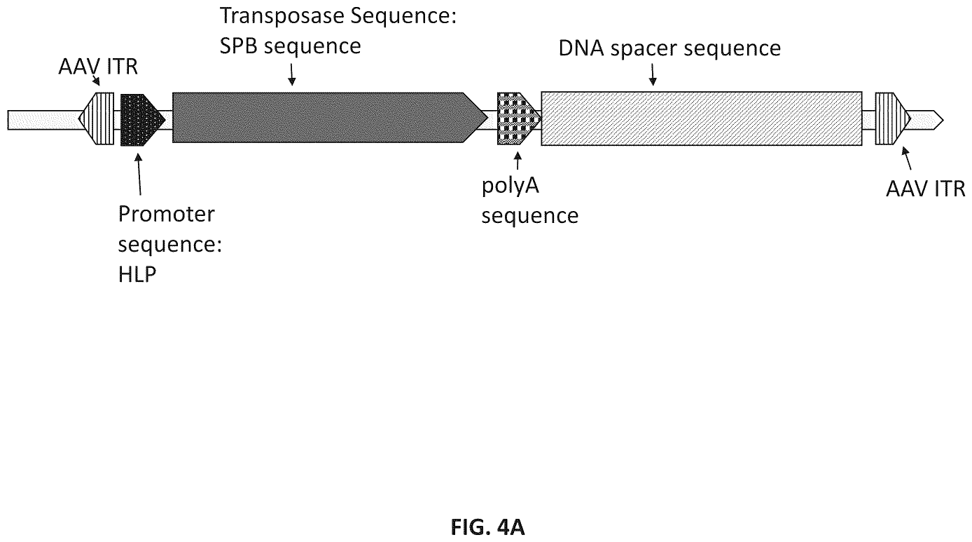

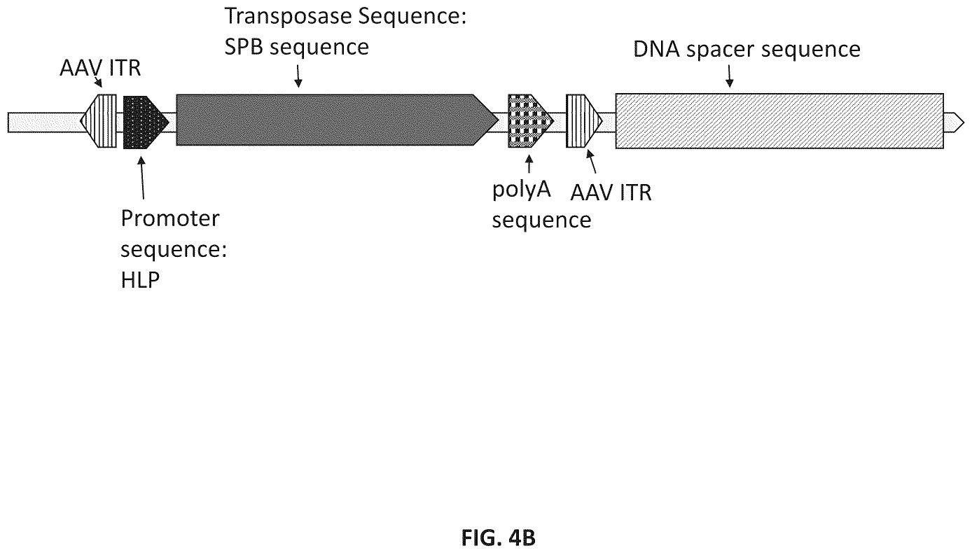

In a non-limiting example of the preceding AAV transposase polynucleotides, the at least one promoter sequence can comprise a hybrid liver promoter (HLP) and the at least one transposase sequence can comprise a nucleic acid sequence encoding a Super piggyBac™ (SPB) transposase polypeptide. This non-limiting example of an AAV piggyBac transposon polynucleotide is shown in FIG. 4A.

In some aspects, an AAV transposase polynucleotide can comprise, in between a polyA sequence and a second AAV ITR sequence, at least one DNA spacer sequence, as is shown in the non-limiting example presented in FIG. 4A.

In some aspects, an AAV transposase polynucleotide can comprise a first AAV ITR sequence, at least one promoter sequence, at least one transposase sequence, a polyA sequence, a second AAV ITR sequence and at least one DNA spacer sequence.

In some aspects, an AAV transposase polynucleotide can comprise in the 5′ to 3′ direction a first AAV ITR sequence, at least one promoter sequence, at least one transposase sequence, a polyA sequence, a second AAV ITR sequence and at least one DNA spacer sequence.

In some aspects, an AAV transposase polynucleotide can comprise a first AAV ITR sequence, followed by at least one promoter sequence, followed by at least one transposase sequence, followed by a polyA sequence, followed by a second AAV ITR sequence and followed by at least one DNA spacer sequence.

In a non-limiting example of the preceding AAV transposase polynucleotides, the at least one promoter sequence can comprise a hybrid liver promoter (HLP) and the at least one transposase sequence can comprise a nucleic acid sequence encoding a Super piggyBac™ (SPB) transposase polypeptide. This non-limiting example of an AAV piggyBac transposon polynucleotide is shown in FIG. 4B.

In some aspects, an AAV transposase polynucleotide can comprise, after a second AAV ITR sequence, at least one DNA spacer sequence, as is shown in the non-limiting example presented in FIG. 4B.

In some aspects, an AAV transposase polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 110.

In some aspects, an AAV transposase polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 144.

### Transposase Sequences

In some aspects, a transposase sequence can comprise a nucleic acid sequence that encodes for any transposase polypeptide known in the art. In some aspects, a transposase sequence can comprise a nucleic acid sequence that encodes for a piggyBac™ (PB) transposase polypeptide. In some aspects, a transposase sequence can comprise a nucleic acid sequence that encodes for a piggyBac-like (PBL) transposase polypeptide. In some aspects, a transposase sequence can comprise a nucleic acid sequence that encodes for a Super piggyBac™ (SPB) transposase polypeptide.

Non-limiting examples of PB transposons and PB, PBL and SPB transposases are described in detail in U.S. Pat. Nos. 6,218,182; 6,962,810; 8,399,643 and PCT Publication No. WO 2010/099296.

The PB, PBL and SPB transposases recognize transposon-specific inverted terminal repeat sequences (ITRs) on the ends of the transposon, and inserts the contents between the ITRs at the sequence 5′-TTAA-3′ within a chromosomal site (a TTAA target sequence). The target sequence of the PB or PBL transposon can comprise or consist of 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′. The PB or PBL transposon system has no payload limit for the genes of interest that can be included between the ITRs.

Exemplary amino acid sequences for one or more PB, PBL and SPB transposases are disclosed in U.S. Pat. Nos. 6,218,185; 6,962,810 and 8,399,643. In a preferred aspect, the PB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 39.

The PB or PBL transposase can comprise or consist of an amino acid sequence having an amino acid substitution at two or more, at three or more or at each of positions 30, 165, 282, and/or 538 of the sequence of SEQ ID NO: 39. The transposase can be a SPB transposase that comprises or consists of the amino acid sequence of the sequence of SEQ ID NO: 39 wherein the amino acid substitution at position 30 can be a substitution of a valine (V) for an isoleucine (I), the amino acid substitution at position 165 can be a substitution of a serine(S) for a glycine (G), the amino acid substitution at position 282 can be a substitution of a valine (V) for a methionine (M), and the amino acid substitution at position 538 can be a substitution of a lysine (K) for an asparagine (N). In a preferred aspect, the SPB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 40.

In certain aspects wherein the transposase comprises the above-described mutations at positions 30, 165, 282 and/or 538, the PB, PBL and SPB transposases can further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 39 or SEQ ID NO: 40 are described in more detail in PCT Publication No. WO 2019/173636 and PCT/US2019/049816.

In a preferred aspect, the PB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 41.

The PB or PBL transposase can comprise or consist of an amino acid sequence having an amino acid substitution at two or more, at three or more or at each of positions 29, 164, 281, and/or 537 of the sequence of SEQ ID NO: 41. The transposase can be a SPB transposase that comprises or consists of the amino acid sequence of the sequence of SEQ ID NO: 41 wherein the amino acid substitution at position 29 can be a substitution of a valine (V) for an isoleucine (I), the amino acid substitution at position 164 can be a substitution of a serine(S) for a glycine (G), the amino acid substitution at position 281 can be a substitution of a valine (V) for a methionine (M), and the amino acid substitution at position 537 can be a substitution of a lysine (K) for an asparagine (N). In a preferred aspect, the SPB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 42.

In certain aspects wherein the transposase comprises the above-described mutations at positions 29, 164, 281, and/or 537, the PB, PBL and SPB transposases can further comprise an amino acid substitution at one or more of positions 2, 45, 81, 102, 118, 124, 176, 179, 184, 186, 199, 206, 208, 225, 234, 239, 240, 242, 257, 295, 297, 310, 314, 318, 326, 327, 339, 420, 435, 455, 469, 485, 502, 551, 569 and 590 of the sequence of SEQ ID NO: 41 or SEQ ID NO: 42 are described in more detail in PCT Publication No. WO 2019/173636 and PCT/US2019/049816.

The PB, PBL or SPB transposases can be isolated or derived from an insect, vertebrate, crustacean or urochordate as described in more detail in PCT Publication No. WO 2019/173636 and PCT/US2019/049816. In preferred aspects, the PB, PBL or SPB transposases is be isolated or derived from the insect Trichoplusia ni (GenBank Accession No. AAA87375) or Bombyx mori (GenBank Accession No. BAD11135).

A hyperactive PB or PBL transposase is a transposase that is more active than the naturally occurring variant from which it is derived. In a preferred aspect, a hyperactive PB or PBL transposase is isolated or derived from Bombyx mori or Xenopus tropicalis. Examples of hyperactive PB or PBL transposases are disclosed in U.S. Pat. Nos. 6,218,185; 6,962,810, 8,399,643 and WO 2019/173636. A list of hyperactive amino acid substitutions is disclosed in U.S. Pat. No. 10,041,077.

In some aspects, a PB, PBL or SPB transposase is integration deficient. An integration deficient PB, PBL or SPB transposase is a transposase that can excise its corresponding transposon, but that integrates the excised transposon at a lower frequency than a corresponding wild type transposase. Examples of integration deficient PB, PBL or SPB transposases are disclosed in U.S. Pat. Nos. 6,218,185; 6,962,810, 8,399,643 and WO 2019/173636. A list of integration deficient amino acid substitutions is disclosed in U.S. Pat. No. 10,041,077.

In some aspects, a PB, PBL or SPB transposase can fused to a nuclear localization signal. Examples of PB, PBL or SPB transposases fused to a nuclear localization signal are disclosed in U.S. Pat. Nos. 6,218,185; 6,962,810, 8,399,643 and WO 2019/173636. A nuclear localization signal can comprise, consist essentially of or consist of a of the amino acid sequence at least 65%, 70%, 75%, 80%, 85%, 90%, 95%, 96%, 97%, 98%, 99% or 100% (or any percentage in between) identical to SEQ ID NO: 43. A nuclear localization signal can be encoded by a nucleic acid sequence that comprises, consists essentially of or consists of the nucleic acid sequence at least 65%, 70%, 75%, 80%, 85%, 90%, 95%, 96%, 97%, 98%, 99% or 100% (or any percentage in between) identical to SEQ ID NO: 44.

In some aspects, a nuclear localization signal can be fused to a PB, PBL or SPB transposase using a G4S linker located between the NLS and the PB, PBL or SPB. A G4S linker can comprise, consist essentially of or consist of an amino acid sequence at least 65%, 70%, 75%, 80%, 85%, 90%, 95%, 96%, 97%, 98%, 99% or 100% (or any percentage in between) identical to SEQ ID NO: 45. A G4S linker can be encoded by a nucleic acid sequence that comprises, consists essentially of or consists of the nucleic acid sequence at least 65%, 70%, 75%, 80%, 85%, 90%, 95%, 96%, 97%, 98%, 99% or 100% (or any percentage in between) identical to SEQ ID NO: 46.

In some aspects, a transposase sequence can comprise a nucleic acid sequence that encodes for a SBP transposase polypeptide fused to an NLS, wherein the SBP transposase polypeptide fused to an NLS comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 47. In some aspects, a nucleic acid sequence that encodes for a SBP transposase polypeptide fused to an NLS can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 48.

In some aspects, a transposase sequence can comprise a nucleic acid sequence that encodes for a SBP transposase polypeptide fused to an NLS, wherein the SBP transposase polypeptide fused to an NLS comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 49. In some aspects, a nucleic acid sequence that encodes for a SBP transposase polypeptide fused to an NLS can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 50.

In some aspects, a transposase sequence can comprise a nucleic acid sequence that encodes for a Sleeping Beauty transposase polypeptide (for example as disclosed in U.S. Pat. No. 9,228,180). In some aspects, a transposase sequence can comprise a nucleic acid sequence that encodes for a Hyperactive Sleeping Beauty (SB100X) transposase polypeptide. In some aspects, a Sleeping Beauty transposase comprises or consists of an amino acid sequence at least 65%, 70%, 75%, 80%, 85%, 90%, 95%, 96%, 97%, 98%, 99% or 100% (or any percentage in between) identical to SEQ ID NO: 51 and 52. In a preferred aspect, hyperactive Sleeping Beauty (SB100X) transposase comprises, consists essentially of or consists of an amino acid sequence at least 65%, 70%, 75%, 80%, 85%, 90%, 95%, 96%, 97%, 98%, 99% or 100% (or any percentage in between) identical to SEQ ID NO: 53 and 54.

In some aspects, a transposase sequence can comprise a nucleic acid sequence that encodes for a helitron transposase polypeptide (for example, as disclosed in WO 2019/173636). In some aspects, a Helitron transposase polypeptide comprises, consists essentially of or consists of an amino acid sequence at least 65%, 70%, 75%, 80%, 85%, 90%, 95%, 96%, 97%, 98%, 99% or 100% (or any percentage in between) identical to SEQ ID NO: 55 or 56.

In some aspects, a transposase sequence can comprise a nucleic acid sequence that encodes for a Tol2 transposase polypeptide (for example, as disclosed in WO 2019/173636). In some aspects, a Tol2 transposase polypeptide comprises, consists essentially of or consists of an amino acid sequence at least 65%, 70%, 75%, 80%, 85%, 90%, 95%, 96%, 97%, 98%, 99% or 100% (or any percentage in between) identical to SEQ ID NO: 57 or 58.

In some aspects, a transposase sequence can comprise a nucleic acid sequence that encodes for a TcBuster transposase polypeptide (for example, as disclosed in WO 2019/173636) or a mutant TcBuster transposase polypeptide (as described in more detail in PCT Publication No. WO 2019/173636 and PCT/US2019/049816). In some aspects, a TcBuster transposase polypeptide comprises, consists essentially of or consists of an amino acid sequence at least 65%, 70%, 75%, 80%, 85%, 90%, 95%, 96%, 97%, 98%, 99% or 100% (or any percentage in between) identical to SEQ ID NO: 59 or 60. The polynucleotide encoding a TcBuster transposase can comprise or consist of a naturally occurring nucleic acid sequence or a non-naturally occurring nucleic acid sequence.

### Nanoplasmids for Testing Liver-Specific Promoters

The present disclosure provides compositions comprising nanoplasmids for testing liver-specific promoters, herein referred to as “liver nanoplasmids”.

In some aspects, a liver nanoplasmid can comprise at least one piggyBac ITR sequence. In some aspects, a liver nanoplasmid can comprise at least one insulator sequence. In some aspects, a liver nanoplasmid can comprise at least one promoter sequence. In some aspects, a liver nanoplasmid can comprise at least one fluorescent protein sequence. In some aspects, a liver nanoplasmid can comprise at least one self-cleaving peptide sequence. In some aspects, a liver nanoplasmid can comprise at least one luciferase sequence. In some aspects, a liver nanoplasmid can comprise at least one polyA sequence.

A liver nanoplasmid can comprise a first piggyBac ITR sequence, a first insulator sequence, at least one promoter sequence, a fluorescent protein sequence, at least one self-cleaving peptide sequence, a luciferase sequence, a polyA sequence, a second insulator sequence and a second piggyBac ITR sequence. In some aspects, a liver nanoplasmid can comprise in the 5′ to 3′ direction a first piggyBac ITR sequence, a first insulator sequence, at least one promoter sequence, a fluorescent protein sequence, at least one self-cleaving peptide sequence, a luciferase sequence, a polyA sequence, a second insulator sequence and a second piggyBac ITR sequence.

### In Some Aspects of the Present Disclosure, a Transgene Sequence can Comprise a Fluorescent Protein Sequence.

In some aspects, a fluorescent protein sequence can comprise a nucleic acid sequence that encodes for an eGFP polypeptide. In some aspects, a fluorescent protein sequence can comprise a nucleic acid sequence that encodes for an eGFP polypeptide, wherein the eGFP polypeptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NOs: 61 or 62. In some aspects, a nucleic acid sequence that encodes for an eGFP polypeptide can comprise, consist essentially of, or consist of a nucleic acid sequence at least 75%, 80%, 85%, 90%, 95%, 96%, 97%, 98%, 99% or 100% (or any percentage in between) identical to any of the sequences put forth in SEQ ID NOs: 63, 64 or 133.

In some aspects of the present disclosure, a transgene sequence can comprise a luciferase sequence.

In some aspects, a luciferase sequence can comprise a nucleic acid sequence that encodes for an fLuc2 polypeptide. In some aspects, a luciferase sequence can comprise a nucleic acid sequence that encodes for an fLuc2 polypeptide, wherein the fLuc2 polypeptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NOs: 65 or 66. In some aspects, a nucleic acid sequence that encodes for an eGFP polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 67 or 68.

In some aspects, a luciferase sequence can comprise a nucleic acid sequence that encodes for a nanoluciferase (nLuc) polypeptide. In some aspects, a nucleic acid sequence that encodes for an nLuc polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 134.

### Vectors of the Present Disclosure

The present disclosure provides compositions comprising a vector, wherein the vector comprises at least one adeno-associated virus (AAV) piggyBac transposon polynucleotide. A vector comprising at least one adeno-associated virus (AAV) piggyBac transposon polynucleotide is herein referred to as an “AAV piggyBac transposon vector”.

The present disclosure provides compositions comprising a vector, wherein the vector comprises at least one AAV transposase polynucleotide. A vector comprising at least one AAV transposase polynucleotide is herein referred to as an “AAV transposase vector”.

A vector of the present disclose can be a viral vector or a recombinant vector. Viral vectors can comprise a sequence isolated or derived from a retrovirus, a lentivirus, an adenovirus, an adeno-associated virus or any combination thereof. The viral vector may comprise a sequence isolated or derived from an adeno-associated virus (AAV). The viral vector may comprise a recombinant AAV (rAAV).

Exemplary adeno-associated viruses and recombinant adeno-associated viruses include, but are not limited to all serotypes (e.g., AAV1, AAV2, AAV3, AAV4, AAV5, AAV6, AAV7, AAV8, AAV9, AAV10 and AAV11). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03, AAV-KP-1 (also referred to as AAV-KP1; described in detail in Kerun et al. JCI Insight, 2019; 4(22):e131610) and AAV-NP59 (described in detail in Paulk et al. Molecular Therapy, 2018; 26(1):289-303).

The present disclosure provides a composition comprising a plurality of AAV-KP-1 particles comprising at least one adeno-associated virus (AAV) piggyBac transposon polynucleotide. The present disclosure provides a composition comprising a plurality of AAV-KP-1 particles comprising at least one AAV transposase polynucleotide. The present disclosure provides a composition comprising a plurality of AAV-KP-1 particles comprising at least one adeno-associated virus (AAV) piggyBac transposon polynucleotide and a plurality of AAV-KP-1 particles comprising at least one AAV transposase polynucleotide.

The present disclosure provides a composition comprising a plurality of AAV-NP59 particles comprising at least one adeno-associated virus (AAV) piggyBac transposon polynucleotide. The present disclosure provides a composition comprising a plurality of AAV-NP59 particles comprising at least one AAV transposase polynucleotide. The present disclosure provides a composition comprising a plurality of AAV-NP59 particles comprising at least one adeno-associated virus (AAV) piggyBac transposon polynucleotide and a plurality of AAV-NP59 particles comprising at least one AAV transposase polynucleotide.

The viral vectors and viral particles of the present disclosure can be produced using standard methods known in the art.

In some aspects, AAV-KP-1 particles of the present disclosure can be produced using a KP-1 capsid vector, wherein the KP-1 capsid vector comprises at least one of the nucleic acid sequences of SEQ ID NO: 70 and SEQ ID NO: 71. In some aspects, AAV-KP-1 particles of the present disclosure can be produced using an AAV vector packaging plasmid, wherein the AAV vector packaging plasmid comprises at least of the nucleic acid sequences of SEQ ID NO: 75 and SEQ ID NO: 76.

In some aspects, AAV-NP59 particles of the present disclosure can be produced using a NP-59 capsid vector, wherein the NP-59 capsid vector comprises at least one of the nucleic acid sequences of SEQ ID NO: 72, SEQ ID NO: 73 and SEQ ID NO: 74. In some aspects, AAV-NP59 particles of the present disclosure can be produced using an AAV vector packaging plasmid, wherein the AAV vector packaging plasmid comprises at least of the nucleic acid sequences of SEQ ID NO: 75 and SEQ ID NO: 76.

The cell delivery compositions (e.g., polynucleotides, vectors) disclosed herein can comprise a nucleic acid encoding a therapeutic protein or therapeutic agent. Examples of therapeutic proteins include those disclosed in PCT Publication No. WO 2019/173636 and PCT/US2019/049816. Therapeutic proteins can also include, but are not limited to, any of polypeptides described herein as part of transgene sequences (e.g. OTC, MUT1, etc.)

### Formulations, Dosages and Modes of Administration

The present disclosure provides formulations, dosages and methods for administration of the compositions described herein.

The disclosed compositions and pharmaceutical compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffinose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means.

A composition of the disclosure can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

For parenteral administration, any composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

Formulations for oral administration rely on the co-administration of adjuvants (e.g., resorcinols and nonionic surfactants, such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. Formulations for delivery of hydrophilic agents including proteins and protein scaffolds and a combination of at least two surfactants intended for oral, buccal, mucosal, nasal, pulmonary, vaginal transmembrane, or rectal administration are described in U.S. Pat. No. 6,309,663. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant, such as magnesium stearate, paraben, preserving agent, such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations can contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. Nos. 5,879,681 and 5,871,753 and used to deliver biologically active agents orally are known in the art.

For pulmonary administration, preferably, a composition or pharmaceutical composition described herein is delivered in a particle size effective for reaching the lower airways of the lung or sinuses. The composition or pharmaceutical composition can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers (e.g., jet nebulizer, ultrasonic nebulizer), dry powder generators, sprayers, and the like. All such devices can use formulations suitable for the administration for the dispensing of a composition or pharmaceutical composition described herein in an aerosol. Such aerosols can be comprised of either solutions (both aqueous and non-aqueous) or solid particles. Additionally, a spray including a composition or pharmaceutical composition described herein can be produced by forcing a suspension or solution of at least one protein scaffold through a nozzle under pressure. In a metered dose inhaler (MDI), a propellant, a composition or pharmaceutical composition described herein, and any excipients or other additives are contained in a canister as a mixture including a liquefied compressed gas. Actuation of the metering valve releases the mixture as an aerosol, preferably containing particles in the size range of less than about 10 μm, preferably, about 1 μm to about 5 μm, and, most preferably, about 2 μm to about 3 μm. A more detailed description of pulmonary administration, formulations and related devices is disclosed in PCT Publication No. WO 2019/049816.

For absorption through mucosal surfaces, compositions include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the disclosure can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695). A more detailed description of mucosal administration and formulations is disclosed in PCT Publication No. WO 2019/049816.

For transdermal administration, a composition or pharmaceutical composition disclosed herein is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599). A more detailed description of transdermal administration, formulations and suitable devices is disclosed in PCT Publication No. WO 2019/049816.

It can be desirable to deliver the disclosed compounds to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the disclosed compounds or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J. Preferred doses can optionally include about 0.1-99 and/or 100-500 mg/kg/administration, or any range, value or fraction thereof, or to achieve a serum concentration of about 0.1-5000 μg/ml serum concentration per single or multiple administration, or any range, value or fraction thereof. A preferred dosage range for the compositions or pharmaceutical compositions disclosed herein is from about 1 mg/kg, up to about 3, about 6 or about 12 mg/kg of body weight of the subject.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably, 0.1 to 10 milligrams per kilogram per administration or in sustained release form is effective to obtain desired results.

As a non-limiting example, treatment of humans or animals can be provided as a one-time or periodic dosage of the compositions or pharmaceutical compositions disclosed herein about 0.1 to 100 mg/kg or any range, value or fraction thereof per day, on at least one of day 1-40, or, alternatively or additionally, at least one of week 1-52, or, alternatively or additionally, at least one of 1-20 years, or any combination thereof, using single, infusion or repeated doses.

Dosage forms suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

An effective amount can comprise an amount of about 0.001 to about 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

In aspects where the compositions to be administered to a subject in need thereof are modified cells as disclosed herein, the cells can be administered between about 1×10³ and 1×10¹⁵ cells; about 1×10⁴ and 1×10¹² cells; about 1×10⁵ and 1×10¹⁰ cells; about 1×10⁶ and 1×10⁹ cells; about 1×10⁶ and 1×10⁸ cells; about 1×10⁶ and 1×10⁷ cells; or about 1×10⁶ and 25×10⁶ cells. In one aspect the cells are administered between about 5×10⁶ and 25×10⁶ cells.

A more detailed description of pharmaceutically acceptable excipients, formulations, dosages and methods of administration of the disclosed compositions and pharmaceutical compositions is disclosed in PCT Publication No. WO 2019/049816.

### Methods of Using the Compositions of the Disclosure

The present disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. Preferably, the subject is human. The terms “subject” and “patient” are used interchangeably herein.

The disclosure provides a method for treating at least one metabolic liver disorder (MLD) in a subject in need thereof comprising administering to the subject at least one therapeutically effective amount of at least one composition of the present disclosure.

The present disclosure provides at least one composition of the present disclosure for the use in the treatment of at least one metabolic liver disorder in a subject, wherein the at least one composition is for administration to the subject in at least one therapeutically effective amount.

The present disclosure provides the use of at least one composition of the present disclosure for the manufacture of a medicament for the treatment of at least one metabolic liver disorder in a subject, wherein the at least one composition is for administration to the subject in at least one therapeutically effective amount.

In some aspects of the preceding methods and uses, the at least one composition of the present disclosure can comprise at least one AAV piggyBac transposon vector of the present disclosure.

Accordingly, the disclosure provides a method for treating at least one metabolic liver disorder in a subject in need thereof comprising administering to the subject at least one therapeutically effective amount of at least one AAV piggyBac transposon vector of the present disclosure.

The present disclosure provides at least one AAV piggyBac transposon vector of the present disclosure for the use in the treatment of at least one metabolic liver disorder in a subject, wherein the at least one AAV piggyBac transposon vector is for administration to the subject in at least one therapeutically effective amount.

The present disclosure provides the use of at least one AAV piggyBac transposon vector of the present disclosure for the manufacture of a medicament for the treatment of at least one metabolic liver disorder in a subject, wherein the at least one AAV piggyBac transposon vector is for administration to the subject in at least one therapeutically effective amount.

In some aspects of the preceding methods and uses, the at least one composition of the present disclosure can comprise at least one AAV transposase vector of the present disclosure.

Accordingly, the disclosure provides a method for treating at least one metabolic liver disorder in a subject in need thereof comprising administering to the subject at least one therapeutically effective amount of at least one AAV transposase vector of the present disclosure.

The present disclosure provides at least one AAV transposase vector of the present disclosure for the use in the treatment of at least one metabolic liver disorder in a subject, wherein the at least one AAV transposase vector is for administration to the subject in at least one therapeutically effective amount.

The present disclosure provides the use of at least one AAV transposase vector of the present disclosure for the manufacture of a medicament for the treatment of at least one metabolic liver disorder in a subject, wherein the at least one AAV transposase vector is for administration to the subject in at least one therapeutically effective amount.

The present disclosure provides methods of treating at least one metabolic liver disorder in a subject, the methods comprising administering to the subject: a) at least one therapeutically effective amount of a composition comprising a nucleic acid molecule comprising a transposon, wherein the transposon comprises a nucleotide sequence encoding at least one therapeutic protein; and b) at least one therapeutically effective amount of a composition comprising a nucleic acid molecule comprising a nucleotide sequence encoding at least one transposase.

In some aspects of the preceding method, a composition comprising a nucleic acid molecule comprising a transposon can be any AAV piggyBac transposon vector described herein.

In some aspects of the preceding method, a composition comprising a nucleic acid molecule comprising a nucleotide sequence encoding at least one transposase can be any AAV transposase vector of the present disclosure.

Accordingly, the present disclosure provides methods of treating at least one metabolic liver disorder in a subject, the methods comprising administering to the subject: a) at least one therapeutically effective amount of at least one AAV piggyBac transposon vector of the present disclosure; and b) at least one therapeutically effective amount of at least one AAV transposase vector of the present disclosure.

Accordingly the present disclosure provides a combination of at least one AAV piggyBac transposon vector of the present disclosure and at least one AAV transposase vector of the present disclosure for use in in the treatment of at least one metabolic liver disorder in a subject, wherein the at least one AAV piggyBac transposon vector is for administration to the subject in at least one therapeutically effective amount, and wherein the at least one AAV transposase vector is for administration to the subject in at least one therapeutically effective amount.

Accordingly the present disclosure provides the use of a combination of at least one AAV piggyBac transposon vector of the present disclosure and at least one AAV transposase vector of the present disclosure in the manufacture of a medicament for the treatment of at least one metabolic liver disorder in a subject, wherein the at least one AAV piggyBac transposon vector is for administration to the subject in at least one therapeutically effective amount, and wherein the at least one AAV transposase vector is for administration to the subject in at least one therapeutically effective amount.

Metabolic liver disorders can include, but are not limited to, urea cycle disorders, N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome) methylmalonic acidemia (MMA), progressive familial intrahepatic cholestasis type 1 (PFIC1), progressive familial intrahepatic cholestasis type 1 (PFIC2), progressive familial intrahepatic cholestasis type 1 (PFIC3) or any combination thereof. In some aspects, the metabolic liver disorder is Ornithine Transcarbamylase (OTC) Deficiency.

In some aspects of the preceding methods, a composition comprising a nucleic acid molecule comprising a transposon, wherein the transposon comprises a nucleotide sequence encoding at least one therapeutic protein and a composition comprising a nucleic acid molecule comprising a nucleotide sequence encoding at least one transposase can be administered concurrently. In some aspects, a composition comprising a nucleic acid molecule comprising a transposon, wherein the transposon comprises a nucleotide sequence encoding at least one therapeutic protein and a composition comprising a nucleic acid molecule comprising a nucleotide sequence encoding at least one transposase can be administered sequentially. In some aspects, a composition comprising a nucleic acid molecule comprising a transposon, wherein the transposon comprises a nucleotide sequence encoding at least one therapeutic protein and a composition comprising a nucleic acid molecule comprising a nucleotide sequence encoding at least one transposase can be administered in temporal proximity.

As used herein, the term “temporal proximity” refers to that administration of one therapeutic composition (e.g., a composition comprising a transposon) occurs within a time period before or after the administration of another therapeutic composition (e.g., a composition comprising a transposase), such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

In some aspects of the treatment methods of the present disclosure, the administration of the at least one composition and/or vector of the present disclosure to a subject can result in the expression of an exogenous protein (e.g. a therapeutic protein, a transposase, etc.) in at least one organ and/or tissue in the subject.

In some aspects, the administration of the at least one composition and/or vector of the present disclosure results in the expression of the exogenous protein in at least about 10%, or at least about 15%, or at least bout 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99% of the cells in the tissue and/or organ.

In some aspects, the administration of the at least one composition and/or vector of the present disclosure results in the expression of the exogenous protein in at least about 10%, or at least about 15%, or at least bout 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99% of a specific subset or subsets of cells in the tissue and/or organ.

In some aspects, the administration of the at least one composition and/or vector of the present disclosure results in the expression of the exogenous protein for at least about 1 day, or at least about 2 days, or at least about 3 days, or at least about 4 days, or at least about 5 days, or at least about 6 days, or at least about 7 days, or at least about 8 days, or at least about 9 days, or at least about 10 days in the tissue and/or organ.

In some aspects, the administration of the at least one composition and/or vector of the present disclosure results in the expression of the exogenous protein for at least about 1 day, or at least about 2 days, or at least about 3 days, or at least about 4 days, or at least about 5 days, or at least about 6 days, or at least about 7 days, or at least about 8 days, or at least about 9 days, or at least about 10 days in a specific subset or subsets of cells in the tissue and/or organ.

In some aspects, the administration of the at least one composition and/or vector of the present disclosure results in the expression of the exogenous protein for no more than about 1 day, or no more than about 2 days, or no more than about 3 days, or no more than about 4 days, or no more than about 5 days, or no more than about 6 days, or no more than about 7 days, or no more than about 8 days, or no more than about 9 days, or no more than about 10 days in the tissue and/or organ.

In some aspects, the administration of the at least one composition and/or vector of the present disclosure results in the expression of the exogenous protein for no more than about 1 day, or no more than about 2 days, or no more than about 3 days, or no more than about 4 days, or no more than about 5 days, or no more than about 6 days, or no more than about 7 days, or no more than about 8 days, or no more than about 9 days, or no more than about 10 days in a specific subset or subsets of cells in the tissue and/or organ.

In some aspects, the tissue and/or organ can be the liver. In some aspects, the specific subset or subsets of cells can include, but are not limited to, hepatocytes, a hepatic stellate cells, Kupffer cells, liver sinusoidal endothelial cells or any combination thereof.

Any method of the present disclosure can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering, before concurrently, and/or after, at least one additional treatment for urea cycle disorders.

Additional treatments for urea cycle disorders can include, but are not limited to dialysis, hemofiltration, caloric supplementation, hormonal suppression, glucose drip, insulin drip, pharmacologic scavenging of excess nitrogen, administration of dextrose, administration of fluids, administration of Intralipid®, administration of ammonia scavengers, administration of arginine, administration of sodium phenylacetate, administration of sodium benzoate, administration of Ammonul, administration of phnylbutyrate, citrulline supplementation, arginine supplementation, or any combination thereof.

### EXEMPLARY EMBODIMENTS OF THE PRESENT DISCLOSURE

Embodiment 1. An adeno-associated virus (AAV) piggyBac transposon polynucleotide comprising in the 5′ to 3′ direction:
a) a first AAV inverted terminal repeat (ITR) sequence;
b) a first piggyBac ITR sequence;
c) a first insulator sequence;
d) at least one promoter sequence;
e) at least one transgene sequence;
f) a polyA sequence;
g) a second insulator sequence;
h) a second piggyBac ITR sequence; and
i) a second AAV ITR sequence.

Embodiment 2. The AAV piggyBac transposon polynucleotide of embodiment 1, wherein the AAV piggyBac transposon polynucleotide comprises DNA, cDNA, gDNA, RNA, mRNA or any combination thereof.

Embodiment 3. The AAV piggyBac transposon polynucleotide of any of the preceding embodiments, wherein the first and/or the second AAV ITR sequence comprises the nucleic acid sequence of any one of SEQ ID NOs: 1-4, 93-94, 105-106 and 127.

Embodiment 4. The AAV piggyBac transposon polynucleotide of any of the preceding embodiments, wherein the first AAV ITR sequence comprises the nucleic acid sequence of SEQ ID NO: 3 and the second AAV ITR sequence comprises the nucleic acid sequence of SEQ ID NO: 4.

Embodiment 5. The AAV piggyBac transposon polynucleotide of any of the preceding embodiments, wherein the first piggyBac ITR sequence and/or the second piggyBac ITR sequence comprises the nucleic acid sequence of any one of SEQ ID NOs: 5-6, 86-90, 95-96 and 125.

Embodiment 6. The AAV piggyBac transposon polynucleotide of any of the preceding embodiments, wherein the first piggyBac ITR sequence comprises the nucleic acid sequence of SEQ ID NO: 5 and the second piggyBac ITR comprises the nucleic acid sequence of SEQ ID NO: 6.

Embodiment 7. The AAV piggyBac transposon polynucleotide of any of the preceding embodiments, wherein the first insulator sequence and/or the second insulator sequence comprises the nucleic acid sequence of any one of SEQ ID NOs: 7-8, 77-80 and 91-92.

Embodiment 8. The AAV piggyBac transposon polynucleotide of any of the preceding embodiments, wherein the first insulator sequence comprises the nucleic acid sequence of SEQ ID NO: 7 and the second insulator sequence comprises the nucleic acid sequence of SEQ ID NO: 8.

Embodiment 9. The AAV piggyBac transposon polynucleotide of any of the preceding embodiments, wherein the at least one promoter sequence is a liver-specific promoter.

Embodiment 10. The AAV piggyBac transposon polynucleotide of any of the preceding embodiments, wherein the liver-specific promoter is a hybrid liver promoter (HLP), an LP1 promoter, a leukocyte-specific expression of the pp52 (LSP1) long promoter, a thyroxine binding globulin (TBG) promoter, a wTBG promoter, a hepatic combinatorial bundle (HCB) promoter, a 2×ApoE-hAAT promoter or a leukocyte-specific expression of the pp52 (LSP1) plus chimeric intron promoter.

Embodiment 11. The AAV piggyBac transposon polynucleotide of any of the preceding embodiments, wherein the at least one promoter sequence comprises the nucleic acid sequence of any of SEQ IDS NOs: 9-16, 69, 107, 126 and 132.

Embodiment 12. The AAV piggyBac transposon polynucleotide of any of the preceding embodiments, wherein the at least one transgene sequence comprises a nucleic acid sequence encoding a methylmalonyl-CoA mutase (MUT1) polypeptide.

Embodiment 13. The AAV piggyBac transposon polynucleotide of any of the preceding embodiments, wherein the MUT1 polypeptide comprises the amino acid sequence of SEQ ID NO: 17, 18, 121 or 122.

Embodiment 14. The AAV piggyBac transposon polynucleotide of any of the preceding embodiments, wherein the nucleic acid sequence encoding a MUT1 polypeptide comprises the nucleic acid sequence of SEQ ID NO: 19, 20 or 111-120.

Embodiment 15. The AAV piggyBac transposon polynucleotide of any of the preceding embodiments, wherein the at least one transgene sequence comprises a nucleic acid sequence encoding an ornithine transcarbamylase (OTC) polypeptide.

Embodiment 16. The AAV piggyBac transposon polynucleotide of any of the preceding embodiments, wherein the OTC polypeptide comprises the amino acid sequence of SEQ ID NO: 21 or 81.

Embodiment 17. The AAV piggyBac transposon polynucleotide of any of the preceding embodiments, wherein the nucleic acid sequence encoding an OTC polypeptide comprises the nucleic acid sequence of any of SEQ ID NOs: 22, 23, 82 and 83.

Embodiment 18. The AAV piggyBac transposon polynucleotide of any of the preceding embodiments, wherein the at least one transgene sequence comprises a nucleic acid sequence encoding an iCas9 polypeptide.

Embodiment 19. The AAV piggyBac transposon polynucleotide of any of the preceding embodiments, wherein the iCas9 polypeptide comprises the amino acid sequence of SEQ ID NO: 24 or 84.

Embodiment 20. The AAV piggyBac transposon polynucleotide of any of the preceding embodiments, wherein the nucleic acid sequence encoding an iCas9 polypeptide comprises the nucleic acid sequence of SEQ ID NO: 25 or 85.

Embodiment 21. The AAV piggyBac transposon polynucleotide of any of the preceding embodiments, wherein the at least one transgene sequence is operatively linked to the at least one promoter sequence.

Embodiment 22. The AAV piggyBac transposon polynucleotide of any of the preceding embodiments, wherein the expression of the at least one transgene sequence is controlled by the at least one promoter sequence.

Embodiment 23. The AAV piggyBac transposon polynucleotide of any of the preceding embodiments, wherein the polyA sequence comprises the nucleic acid sequence of any one of SEQ ID NO: 26-27, 97, 108, 128 and 136.

Embodiment 24. The AAV piggyBac transposon polynucleotide of any of the preceding embodiments, wherein the AAV piggyBac transposon polynucleotide further comprises at least a second transgene sequence.

Embodiment 25. The AAV piggyBac transposon polynucleotide of any of the preceding embodiments, wherein the at least second transgene sequence comprises a nucleic acid sequence encoding an iCas9 polypeptide.

Embodiment 26. The AAV piggyBac transposon polynucleotide of any of the preceding embodiments, wherein the iCas9 polypeptide comprises the amino acid sequence of SEQ ID NO: 24 or 84.

Embodiment 27. The AAV piggyBac transposon polynucleotide of any of the preceding embodiments, wherein the nucleic acid sequence encoding an iCas9 polypeptide comprises the nucleic acid sequence of SEQ ID NO: 25 or 85.

Embodiment 28. The AAV piggyBac transposon polynucleotide of any of the preceding embodiments, wherein the at least second transgene sequence comprises a nucleic acid sequence encoding a methylmalonyl-CoA mutase (MUT1) polypeptide.

Embodiment 29. The AAV piggyBac transposon polynucleotide of any of the preceding embodiments, wherein the MUT1 polypeptide comprises the amino acid sequence of SEQ ID NO: 17, 18, 121 or 122.

Embodiment 30. The AAV piggyBac transposon polynucleotide of any of the preceding embodiments, wherein the nucleic acid sequence encoding a MUT1 polypeptide comprises the nucleic acid sequence of SEQ ID NO: 19, 20 or 111-120.

Embodiment 31. The AAV piggyBac transposon polynucleotide of any of the preceding embodiments, wherein the at least second transgene sequence comprises a nucleic acid sequence encoding an ornithine transcarbamylase (OTC) polypeptide.

Embodiment 32. The AAV piggyBac transposon polynucleotide of any of the preceding embodiments, wherein the OTC polypeptide comprises the amino acid sequence of SEQ ID NO: 21 or 81.

Embodiment 33. The AAV piggyBac transposon polynucleotide of any of the preceding embodiments, wherein the nucleic acid sequence encoding an OTC polypeptide comprises the nucleic acid sequence of any of SEQ ID NOs: 22, 23, 82 and 83.

Embodiment 34. The AAV piggyBac transposon polynucleotide of any of the preceding embodiments, wherein the AAV piggyBac transposon polynucleotide further comprises at least a second promoter sequence.

Embodiment 35. The AAV piggyBac transposon polynucleotide of any of the preceding embodiments, wherein the at least second promoter sequence is located between the at least one transgene sequence and the at least second transgene sequence.

Embodiment 36. The AAV piggyBac transposon polynucleotide of any of the preceding embodiments, wherein the AAV piggyBac transposon polynucleotide further comprises at least one self-cleaving peptide sequence, wherein the at least one self-cleaving peptide sequence is a nucleic acid sequence encoding for a T2A peptide, GSG-T2A peptide, an E2A peptide, a GSG-E2A peptide, an F2A peptide, a GSG-F2A peptide, a P2A peptide, or a GSG-P2A peptide.

Embodiment 37. The AAV piggyBac transposon polynucleotide of any of the preceding embodiments, wherein the at least one self-cleaving peptide sequence is located between the at least one transgene sequence and the at least second transgene sequence.

Embodiment 38. The AAV piggyBac transposon polynucleotide of any of the preceding embodiments, wherein the AAV piggyBac transposon polynucleotide comprises at least two transgene sequences.

Embodiment 39. The AAV piggyBac transposon polynucleotide of any of the preceding embodiments, wherein the at least two transgene sequences are the same sequence.

Embodiment 40. The AAV piggyBac transposon polynucleotide of any of the preceding embodiments, wherein the at least two transgene sequences are different sequences.

Embodiment 41. The AAV piggyBac transposon polynucleotide of any of the preceding embodiments, further comprising at least one DNA spacer sequence.

Embodiment 42. The AAV piggyBac transposon polynucleotide of any of the preceding embodiments, wherein the at least one DNA spacer sequence comprises the nucleic acid sequence of any one of SEQ ID NO: 103, 109, 129-131 and 137.

Embodiment 43. The AAV piggyBac transposon polynucleotide of any of the preceding embodiments, wherein the AAV piggyBac transposon polynucleotide comprises at least two promoter sequences.

Embodiment 44. The AAV piggyBac transposon polynucleotide of any of the preceding embodiments, wherein the at least two promoter sequences are the same sequence.

Embodiment 45. The AAV piggyBac transposon polynucleotide of any of the preceding embodiments, wherein the at least two promoter sequences are different sequences.

Embodiment 46A. An AAV piggyBac transposon polynucleotide comprising in the 5′ to 3′ direction:
a) a first AAV ITR sequence;
b) a first piggyBac ITR sequence;
c) a first insulator sequence;
d) at least one promoter sequence;
e) at least one transgene sequence;
f) a polyA sequence;
g) a second insulator sequence;
h) a second piggyBac ITR sequence;
i) at least one DNA spacer sequence; and
j) a second AAV ITR sequence.

Embodiment 46B. The AAV piggyBac transposon polynucleotide of any of the preceding embodiments, wherein the first AAV ITR sequence comprises the nucleic acid sequence of SEQ ID NO: 3.

Embodiment 46C. The AAV piggyBac transposon polynucleotide of any of the preceding embodiments, wherein the first piggyBac ITR sequence comprises the nucleic acid sequence of SEQ ID NO: 95.

Embodiment 46D. The AAV piggyBac transposon polynucleotide of any of the preceding embodiments, wherein the first piggyBac ITR sequence comprises the nucleic acid sequence of SEQ ID NO: 125.

Embodiment 46E. The AAV piggyBac transposon polynucleotide of any of the preceding embodiments, wherein the first insulator sequence comprises the nucleic acid sequence of SEQ ID NO: 7.

Embodiment 46F. The AAV piggyBac transposon polynucleotide of any of the preceding embodiments, wherein the at least one promoter sequence comprises the nucleic acid sequence of SEQ ID NO: 9.

Embodiment 46G. The AAV piggyBac transposon polynucleotide of any of the preceding embodiments, wherein the at least one promoter sequence comprises the nucleic acid sequence of SEQ ID NO: 126.

Embodiment 46H. The AAV piggyBac transposon polynucleotide of any of the preceding embodiments, wherein the at least one transgene sequence comprises the nucleic acid sequence of SEQ ID NO: 22.

Embodiment 46I. The AAV piggyBac transposon polynucleotide of any of the preceding embodiments, wherein the polyA sequence comprises the nucleic acid sequence of SEQ ID NO: 97.

Embodiment 46J. The AAV piggyBac transposon polynucleotide of any of the preceding embodiments, wherein the second insulator sequence comprises the nucleic acid sequence of SEQ ID NO: 8.

Embodiment 46K. The AAV piggyBac transposon polynucleotide of any of the preceding embodiments, wherein the a second piggyBac ITR sequence comprises the nucleic acid sequence of SEQ ID NO: 96.

Embodiment 46L. The AAV piggyBac transposon polynucleotide of any of the preceding embodiments, wherein the least one DNA spacer sequence comprises the nucleic acid sequence of SEQ ID NO: 129.

Embodiment 46M. The AAV piggyBac transposon polynucleotide of any of the preceding embodiments, wherein the second AAV ITR sequence comprises the nucleic acid sequence of SEQ ID NO: 4.

Embodiment 46N. An AAV piggyBac transposon polynucleotide comprising in the 5′ to 3′ direction:
a) a first AAV ITR sequence comprising the nucleic acid sequence of SEQ ID NO: 3;
b) a first piggyBac ITR sequence comprising the nucleic acid sequence of SEQ ID NO: 95 or SEQ ID NO: 125;
c) a first insulator sequence comprising the nucleic acid sequence of SEQ ID NO: 7;
d) at least one promoter sequence comprising the nucleic acid sequence of SEQ ID NO: 9 or SEQ ID NO: 126;
e) at least one transgene sequence comprising the nucleic acid sequence of SEQ ID NO: 22;
f) a polyA sequence comprising the nucleic acid sequence of SEQ ID NO: 97;
g) a second insulator sequence comprising the nucleic acid sequence of SEQ ID NO: 8;
h) a second piggyBac ITR sequence comprising the nucleic acid sequence of SEQ ID NO: 96;
i) at least one DNA spacer sequence comprising the nucleic acid sequence of SEQ ID NO: 129; and
j) a second AAV ITR sequence comprising the nucleic acid sequence of SEQ ID NO: 4.

Embodiment 47. The AAV piggyBac transposon polynucleotide of any one of Embodiments 46A-46N, wherein the at least one promoter sequence comprises the nucleic acid sequence of SEQ ID NO: 9.

Embodiment 48. The AAV piggyBac transposon polynucleotide of any one of Embodiments 46A-46N, wherein the at least one promoter sequence comprises the nucleic acid sequence of SEQ ID NO: 126.

Embodiment 49. The AAG piggyBac transposon polynucleotide of any one of Embodiments 46A-46N, wherein the first piggyBac ITR sequence comprises the nucleic acid sequence of SEQ ID NO: 95.

Embodiment 50. The AAG piggyBac transposon polynucleotide of any one of Embodiments 46A-46N, wherein the first piggyBac ITR sequence comprises the nucleic acid sequence of SEQ ID NO: 125.

Embodiment 51. The AAV piggyBac transposon polynucleotide of any one of Embodiments 46A-50, wherein the AAV piggyBac transposon polynucleotide comprises the nucleic acid sequence of SEQ ID NO: 138.

Embodiment 52A. An AAV piggyBac transposon polynucleotide comprising in the 5′ to 3′ direction:
a) a first AAV ITR sequence;
b) a first piggyBac ITR sequence;
c) a first insulator sequence;
d) at least one promoter sequence;
e) at least one transgene sequence;
f) a polyA sequence;
g) a second insulator sequence;
h) a second piggyBac ITR sequence;
i) at least one DNA spacer sequence; and
j) a second AAV ITR sequence.

Embodiment 52B. The AAV piggyBac transposon polynucleotide of any of the preceding embodiments, wherein the first AAV ITR sequence comprises the nucleic acid sequence of SEQ ID NO: 3.

Embodiment 52C. The AAV piggyBac transposon polynucleotide of any of the preceding embodiments, wherein the first piggyBac ITR sequence comprises the nucleic acid sequence of SEQ ID NO: 95.

Embodiment 52D. The AAV piggyBac transposon polynucleotide of any of the preceding embodiments, wherein the first piggyBac ITR sequence comprises the nucleic acid sequence of SEQ ID NO: 125.

Embodiment 52E. The AAV piggyBac transposon polynucleotide of any of the preceding embodiments, wherein the first insulator sequence comprises the nucleic acid sequence of SEQ ID NO: 7.

Embodiment 52F. The AAV piggyBac transposon polynucleotide of any of the preceding embodiments, wherein the at least one promoter sequence comprises the nucleic acid sequence of SEQ ID NO: 10.

Embodiment 52G. The AAV piggyBac transposon polynucleotide of any of the preceding embodiments, wherein the at least one promoter sequence comprises the nucleic acid sequence of SEQ ID NO: 132.

Embodiment 52H. The AAV piggyBac transposon polynucleotide of any of the preceding embodiments, wherein the at least one transgene sequence comprises the nucleic acid sequence of SEQ ID NO: 22.

Embodiment 52I. The AAV piggyBac transposon polynucleotide of any of the preceding embodiments, wherein the polyA sequence comprises the nucleic acid sequence of SEQ ID NO: 97.

Embodiment 52J. The AAV piggyBac transposon polynucleotide of any of the preceding embodiments, wherein the second insulator sequence comprises the nucleic acid sequence of SEQ ID NO: 8.

Embodiment 52K. The AAV piggyBac transposon polynucleotide of any of the preceding embodiments, wherein the a second piggyBac ITR sequence comprises the nucleic acid sequence of SEQ ID NO: 96.

Embodiment 52L. The AAV piggyBac transposon polynucleotide of any of the preceding embodiments, wherein the least one DNA spacer sequence comprises the nucleic acid sequence of SEQ ID NO: 130.

Embodiment 52M. The AAV piggyBac transposon polynucleotide of any of the preceding embodiments, wherein the second AAV ITR sequence comprises the nucleic acid sequence of SEQ ID NO: 4.

Embodiment 52N. AAV piggyBac transposon polynucleotide comprising in the 5′ to 3′ direction:
a) a first AAV ITR sequence comprising the nucleic acid sequence of SEQ ID NO: 3;
b) a first piggyBac ITR sequence comprising the nucleic acid sequence of SEQ ID NO: 95 or SEQ ID NO: 125;
c) a first insulator sequence comprising the nucleic acid sequence of SEQ ID NO: 7;
d) at least one promoter sequence comprising the nucleic acid sequence of SEQ ID NO: 10 or SEQ ID NO: 132;
e) at least one transgene sequence comprising the nucleic acid sequence of SEQ ID NO: 22;
f) a polyA sequence comprising the nucleic acid sequence of SEQ ID NO: 97;
g) a second insulator sequence comprising the nucleic acid sequence of SEQ ID NO: 8;
h) a second piggyBac ITR sequence comprising the nucleic acid sequence of SEQ ID NO: 96;
i) at least one DNA spacer sequence comprising the nucleic acid sequence of SEQ ID NO: 130; and
j) a second AAV ITR sequence comprising the nucleic acid sequence of SEQ ID NO: 4.

Embodiment 53. The AAV piggyBac transposon polynucleotide of any one of Embodiments 52A-52N, wherein the at least one promoter sequence comprises the nucleic acid sequence of SEQ ID NO: 10.

Embodiment 54. The AAV piggyBac transposon polynucleotide of any one of Embodiments 52A-52N, wherein the at least one promoter sequence comprises the nucleic acid sequence of SEQ ID NO: 132.

Embodiment 55. The AAG piggyBac transposon polynucleotide of any one of Embodiments 52A-52N, wherein the first piggyBac ITR sequence comprises the nucleic acid sequence of SEQ ID NO: 95.

Embodiment 56. The AAG piggyBac transposon polynucleotide of any one of Embodiments 52A-52N, wherein the first piggyBac ITR sequence comprises the nucleic acid sequence of SEQ ID NO: 125.

Embodiment 57. The AAV piggyBac transposon polynucleotide of any one of Embodiments 52A-56, wherein the AAV piggyBac transposon polynucleotide comprises the nucleic acid sequence of SEQ ID NO: 139.

Embodiment 58A. An AAV piggyBac transposon polynucleotide comprising in the 5′ to 3′ direction:
a) a first AAV ITR sequence;
b) a first piggyBac ITR sequence;
c) a first insulator sequence;
d) at least one promoter sequence;
e) at least one transgene sequence;
f) a polyA sequence;
g) a second insulator sequence;
h) a second piggyBac ITR sequence;
i) at least one DNA spacer sequence; and
j) a second AAV ITR sequence.

Embodiment 58B. The AAV piggyBac transposon polynucleotide of any of the preceding embodiments, wherein the first AAV ITR sequence comprises the nucleic acid sequence of SEQ ID NO: 3.

Embodiment 58C. The AAV piggyBac transposon polynucleotide of any of the preceding embodiments, wherein the first piggyBac ITR sequence comprises the nucleic acid sequence of SEQ ID NO: 95.

Embodiment 58D. The AAV piggyBac transposon polynucleotide of any of the preceding embodiments, wherein the first piggyBac ITR sequence comprises the nucleic acid sequence of SEQ ID NO: 125.

Embodiment 58E. The AAV piggyBac transposon polynucleotide of any of the preceding embodiments, wherein the first insulator sequence comprises the nucleic acid sequence of SEQ ID NO: 7.

Embodiment 58F. The AAV piggyBac transposon polynucleotide of any of the preceding embodiments, wherein the at least one promoter sequence comprises the nucleic acid sequence of SEQ ID NO: 13.

Embodiment 58G. The AAV piggyBac transposon polynucleotide of any of the preceding embodiments, wherein the at least one transgene sequence comprises the nucleic acid sequence of SEQ ID NO: 22.

Embodiment 58H. The AAV piggyBac transposon polynucleotide of any of the preceding embodiments, wherein the polyA sequence comprises the nucleic acid sequence of SEQ ID NO: 97.

Embodiment 58I. The AAV piggyBac transposon polynucleotide of any of the preceding embodiments, wherein the second insulator sequence comprises the nucleic acid sequence of SEQ ID NO: 8.

Embodiment 58J. The AAV piggyBac transposon polynucleotide of any of the preceding embodiments, wherein the a second piggyBac ITR sequence comprises the nucleic acid sequence of SEQ ID NO: 96.

Embodiment 58K. The AAV piggyBac transposon polynucleotide of any of the preceding embodiments, wherein the least one DNA spacer sequence comprises the nucleic acid sequence of SEQ ID NO: 131.

Embodiment 58L. The AAV piggyBac transposon polynucleotide of any of the preceding embodiments, wherein the second AAV ITR sequence comprises the nucleic acid sequence of SEQ ID NO: 4.

Embodiment 58M. AAV piggyBac transposon polynucleotide comprising in the 5′ to 3′ direction:
a) a first AAV ITR sequence comprising the nucleic acid sequence of SEQ ID NO: 3;
b) a first piggyBac ITR sequence comprising the nucleic acid sequence of SEQ ID NO: 95 or SEQ ID NO: 125;
c) a first insulator sequence comprising the nucleic acid sequence of SEQ ID NO: 7;
d) at least one promoter sequence comprising the nucleic acid sequence of SEQ ID NO: 13;
e) at least one transgene sequence comprising the nucleic acid sequence of SEQ ID NO: 22;
f) a polyA sequence comprising the nucleic acid sequence of SEQ ID NO: 97;
g) a second insulator sequence comprising the nucleic acid sequence of SEQ ID NO: 8;
h) a second piggyBac ITR sequence comprising the nucleic acid sequence of SEQ ID NO: 96;
i) at least one DNA spacer sequence comprising the nucleic acid sequence of SEQ ID NO: 131; and
j) a second AAV ITR sequence comprising the nucleic acid sequence of SEQ ID NO: 4.

Embodiment 59. The AAG piggyBac transposon polynucleotide of any one of Embodiments 58A-58M, wherein the first piggyBac ITR sequence comprises the nucleic acid sequence of SEQ ID NO: 95.

Embodiment 60. The AAG piggyBac transposon polynucleotide of any one of Embodiments 58A-58M, wherein the first piggyBac ITR sequence comprises the nucleic acid sequence of SEQ ID NO: 125.

Embodiment 61. The AAV piggyBac transposon polynucleotide of any one of Embodiments 58A-60, wherein the AAV piggyBac transposon polynucleotide comprises the nucleic acid sequence of SEQ ID NO: 140.

Embodiment 62. An AAV piggyBac transposon polynucleotide comprising in the 5′ to 3′ direction:
a) a first AAV ITR sequence comprising the nucleic acid sequence of SEQ ID NO: 3;
b) a first piggyBac ITR sequence comprising the nucleic acid sequence of SEQ ID NO: 95 or SEQ ID NO: 125;
c) a first insulator sequence comprising the nucleic acid sequence of SEQ ID NO: 7;
d) at least one promoter sequence comprising the nucleic acid sequence of SEQ ID NO: 9 or SEQ ID NO: 126;
e) a first transgene sequence comprising the nucleic acid sequence of SEQ ID NO: 22;
f) a first self-cleaving peptide sequence comprising the nucleic acid sequence of SEQ ID NO: 31;
g) a second transgene sequence comprising the nucleic acid sequence of SEQ ID NO: 133;
h) an at least second self-cleaving peptide sequence comprising the nucleic acid sequence of SEQ ID NO: 32;
i) at least a third transgene sequence comprising the nucleic acid sequence of SEQ ID NO: 134;
j) a polyA sequence comprising the nucleic acid sequence of SEQ ID NO: 97;
k) a second insulator sequence comprising the nucleic acid sequence of SEQ ID NO: 8;
l) a second piggyBac ITR sequence comprising the nucleic acid sequence of SEQ ID NO: 96; and
m) a second AAV ITR sequence comprising the nucleic acid sequence of SEQ ID NO: 4.

Embodiment 63. The AAG piggyBac transposon polynucleotide of Embodiment 62, wherein the first piggyBac ITR sequence comprises the nucleic acid sequence of SEQ ID NO: 95.

Embodiment 64. The AAG piggyBac transposon polynucleotide of Embodiment 62, wherein the first piggyBac ITR sequence comprises the nucleic acid sequence of SEQ ID NO: 125.

Embodiment 65. The AAV piggyBac transposon polynucleotide of Embodiment 62, wherein the at least one promoter sequence comprises the nucleic acid sequence of SEQ ID NO: 9.

Embodiment 66. The AAV piggyBac transposon polynucleotide of Embodiment 62, wherein the at least one promoter sequence comprises the nucleic acid sequence of SEQ ID NO: 126.

Embodiment 67. The AAV piggyBac transposon polynucleotide of any one of Embodiments 62-66, wherein the AAV piggyBac transposon polynucleotide comprises the nucleic acid sequence of SEQ ID NO: 141.

Embodiment 68. An AAV piggyBac transposon polynucleotide comprising in the 5′ to 3′ direction:
a) a first AAV ITR sequence comprising the nucleic acid sequence of SEQ ID NO: 3;
b) a first piggyBac ITR sequence comprising the nucleic acid sequence of SEQ ID NO: 95 or SEQ ID NO: 125;
c) a first insulator sequence comprising the nucleic acid sequence of SEQ ID NO: 7;
d) at least one promoter sequence comprising the nucleic acid sequence of SEQ ID NO: 10 or SEQ ID NO: 132;
e) a first transgene sequence comprising the nucleic acid sequence of SEQ ID NO: 22;
f) a first self-cleaving peptide sequence comprising the nucleic acid sequence of SEQ ID NO: 31;
g) a second transgene sequence comprising the nucleic acid sequence of SEQ ID NO: 133;
h) an at least second self-cleaving peptide sequence comprising the nucleic acid sequence of SEQ ID NO: 32;
i) at least a third transgene sequence comprising the nucleic acid sequence of SEQ ID NO: 134;
j) a polyA sequence comprising the nucleic acid sequence of SEQ ID NO: 97;
k) a second insulator sequence comprising the nucleic acid sequence of SEQ ID NO: 8;
l) a second piggyBac ITR sequence comprising the nucleic acid sequence of SEQ ID NO: 96; and
m) a second AAV ITR sequence comprising the nucleic acid sequence of SEQ ID NO: 4.

Embodiment 69. The AAG piggyBac transposon polynucleotide of Embodiment 68, wherein the first piggyBac ITR sequence comprises the nucleic acid sequence of SEQ ID NO: 95.

Embodiment 70. The AAG piggyBac transposon polynucleotide of Embodiment 68, wherein the first piggyBac ITR sequence comprises the nucleic acid sequence of SEQ ID NO: 125.

Embodiment 71. The AAV piggyBac transposon polynucleotide of Embodiment 68, wherein the at least one promoter sequence comprises the nucleic acid sequence of SEQ ID NO: 10.

Embodiment 72. The AAV piggyBac transposon polynucleotide of Embodiment 68, wherein the at least one promoter sequence comprises the nucleic acid sequence of SEQ ID NO: 132.

Embodiment 73. The AAV piggyBac transposon polynucleotide of any one of Embodiments 68-72, wherein the AAV piggyBac transposon polynucleotide comprises the nucleic acid sequence of SEQ ID NO: 142.

Embodiment 74. An AAV piggyBac transposon polynucleotide comprising in the 5′ to 3′ direction:
a) a first AAV ITR sequence comprising the nucleic acid sequence of SEQ ID NO: 3;
b) a first piggyBac ITR sequence comprising the nucleic acid sequence of SEQ ID NO: 95 or SEQ ID NO: 125;
c) a first insulator sequence comprising the nucleic acid sequence of SEQ ID NO: 7;
d) at least one promoter sequence comprising the nucleic acid sequence of SEQ ID NO: 13;
e) a first transgene sequence comprising the nucleic acid sequence of SEQ ID NO: 22;
f) at least one self-cleaving peptide sequence comprising the nucleic acid sequence of SEQ ID NO: 135;
g) an at least second transgene sequence comprising the nucleic acid sequence of SEQ ID NO: 134;
h) a polyA sequence comprising the nucleic acid sequence of SEQ ID NO: 97;
i) a second insulator sequence comprising the nucleic acid sequence of SEQ ID NO: 8;
j) a second piggyBac ITR sequence comprising the nucleic acid sequence of SEQ ID NO: 96; and
k) a second AAV ITR sequence comprising the nucleic acid sequence of SEQ ID NO: 4.

Embodiment 75. The AAG piggyBac transposon polynucleotide of Embodiment 74, wherein the first piggyBac ITR sequence comprises the nucleic acid sequence of SEQ ID NO: 95.

Embodiment 76. The AAG piggyBac transposon polynucleotide of Embodiment 74, wherein the first piggyBac ITR sequence comprises the nucleic acid sequence of SEQ ID NO: 125.

Embodiment 77. The AAV piggyBac transposon polynucleotide of any one of Embodiments 74-76, wherein the AAV piggyBac transposon polynucleotide comprises the nucleic acid sequence of SEQ ID NO: 143.

Embodiment 78. A vector comprising the AAV piggyBac transposon polynucleotide of any of the preceding embodiments.

Embodiment 79. The vector of any of the preceding embodiments, wherein the vector is a viral vector.

Embodiment 80. The vector of any of the preceding embodiments, wherein the viral vector is an adeno-associated virus (AAV) viral vector.

Embodiment 81. The vector of any of the preceding embodiments, wherein the AAV viral vector is an AAV1, AAV2, AAV3, AAV4, AAV5, AAV6, AAV7, AAV8, AAV9, AAV10 or AAV11 viral vector.

Embodiment 82. The vector of any of the preceding embodiments, wherein the AAV viral vector is an AAV-KP-1 or AAV-NP59 viral vector, preferably wherein the AAV viral vector is an AAV-KP-1 viral vector.

Embodiment 83. A composition comprising the vector of any of embodiments 78-82.

Embodiment 84. An AAV transposase polynucleotide comprising in the 5′ to 3′ direction a first AAV ITR sequence, at least one promoter sequence, at least one transposase sequence, a polyA sequence and a second AAV ITR sequence.

Embodiment 85. The AAV transposase polynucleotide of embodiment 84, wherein the AAV piggyBac transposon polynucleotide comprises DNA, cDNA, gDNA, RNA, mRNA or any combination thereof.

Embodiment 86. The AAV transposase polynucleotide of any of the preceding embodiments, wherein the first and/or the second AAV ITR sequence comprises the nucleic acid sequence of any of SEQ ID NOs: 1-4, 93-94, 105-106 and 127.

Embodiment 87. The AAV transposase polynucleotide of any of the preceding embodiments, wherein the first AAV ITR sequence comprises the nucleic acid sequence of SEQ ID NO: 1 and the second AAV ITR sequence comprises the nucleic acid sequence of SEQ ID NO: 2.

Embodiment 88. The AAV transposase polynucleotide of any of the preceding embodiments, wherein the first AAV ITR sequence comprises the nucleic acid sequence of SEQ ID NO: 105 and the second AAV ITR sequence comprises the nucleic acid sequence of SEQ ID NO: 106.

Embodiment 89. The AAV transposase polynucleotide of any of the preceding embodiments, wherein the at least one promoter sequence is a liver-specific promoter.

Embodiment 90. The AAV transposase polynucleotide of any of the preceding embodiments, wherein the liver-specific promoter is a hybrid liver promoter (HLP), an LP1 promoter, a leukocyte-specific expression of the pp52 (LSP1) long promoter, a thyroxine binding globulin (TBG) promoter, a wTBG promoter, a hepatic combinatorial bundle (HCB) promoter, a 2×ApoE-hAAT promoter or a leukocyte-specific expression of the pp52 (LSP1) plus chimeric intron promoter.

Embodiment 91. The AAV transposase polynucleotide of any of the preceding embodiments, wherein the at least one promoter sequence comprises the nucleic acid sequence of any of SEQ IDS NOs: 9-16, 69, 107, 126 and 132.

Embodiment 92. The AAV transposase polynucleotide of any of the preceding embodiments, wherein the at least one transposase sequence comprises a nucleic acid sequence encoding a piggyBac™ (PB) transposase polypeptide, a piggyBac-like (PBL) transposase polypeptide or a Super piggyBac™ (SPB) transposase polypeptide.

Embodiment 93. The AAV transposase polynucleotide of any of the preceding embodiments, wherein the at least one transposase sequence comprises a nucleic acid sequence encoding for the amino acid sequence of any of SEQ ID NOs: 39-42, 47 and 49.

Embodiment 94. The AAV transposase polynucleotide of any of the preceding embodiments, wherein the at least one transposase sequence comprises the nucleic acid sequence of SEQ ID NO: 48 or 50.

Embodiment 95. The AAV transposase polynucleotide of any of the preceding embodiments, wherein the at least one transposase sequence comprises a nucleic acid sequence encoding a Sleeping Beauty transposase polypeptide, a Hyperactive Sleeping Beauty (SB100X) transposase polypeptide, a helitron transposase polypeptide, a Tol2 transposase polypeptide, a TcBuster transposase polypeptide or a mutant TcBuster transposase polypeptide.

Embodiment 96. The AAV transposase polynucleotide of any of the preceding embodiments, wherein the at least one transposase sequence comprises a nucleic acid sequence encoding the amino acid sequence of any of SEQ ID NOs: 51-60.

Embodiment 97. The AAV transposase polynucleotide of any of the preceding embodiments, wherein the polyA sequence comprises the nucleic acid sequence of SEQ ID NO: 26-27, 97, or 108.

Embodiment 98. The AAV piggyBac transposon polynucleotide of any of the preceding embodiments, wherein the at least one transposase sequence is operatively linked to the at least one promoter sequence.

Embodiment 99. The AAV piggyBac transposon polynucleotide of any of the preceding embodiments, wherein the expression of the at least one transposase sequence is controlled by the at least one promoter sequence.

Embodiment 100. The AAV transposase polynucleotide of any of the preceding embodiments, wherein the AAV transposase polynucleotide further comprises at least one DNA spacer sequence.

Embodiment 101. The AAV transposase polynucleotide of any of the preceding embodiments, wherein the at least one DNA spacer sequence comprises the nucleic acid sequence of SEQ ID NO: 103 or 109.

Embodiment 102A. An AAV transposase polynucleotide comprising in the 5′ to 3′ direction:
a) a first AAV ITR sequence;
b) at least one promoter sequence at least one promoter sequence;
c) at least one transposase sequence;
d) a polyA sequence;
e) at least one DNA spacer sequence; and
f) a second AAV ITR sequence.

Embodiment 102B. The AAV transposase polynucleotide of any of the preceding embodiments, wherein the first AAV ITR sequence comprises the nucleic acid sequence of SEQ ID NO: 127.

Embodiment 102C. The AAV transposase polynucleotide of any of the preceding embodiments, wherein the at least one promoter sequence comprises the nucleic acid sequence of SEQ ID NO: 9.

Embodiment 102D. The AAV transposase polynucleotide of any of the preceding embodiments, wherein the at least one promoter sequence comprises the nucleic acid sequence of SEQ ID NO: 126.

Embodiment 102E. The AAV transposase polynucleotide of any of the preceding embodiments, wherein the at least one transposase sequence comprises the nucleic acid sequence of SEQ ID NO: 48.

Embodiment 102F. The AAV transposase polynucleotide of any of the preceding embodiments, wherein the polyA sequence comprises the nucleic acid sequence of SEQ ID NO: 136.

Embodiment 102G. The AAV transposase polynucleotide of any of the preceding embodiments, wherein the at least one DNA spacer sequence comprises the nucleic acid sequence of SEQ ID NO: 137.

Embodiment 102H. The AAV transposase polynucleotide of any of the preceding embodiments, wherein the at least one DNA spacer sequence comprises the nucleic acid sequence of SEQ ID NO: 4.

Embodiment 102I. An AAV transposase polynucleotide comprising in the 5′ to 3′ direction:
a) a first AAV ITR sequence comprising the nucleic acid sequence of SEQ ID NO: 127;
b) at least one promoter sequence comprising the nucleic acid sequence of SEQ ID NO: 9 or SEQ ID NO: 126;
c) at least one transposase sequence comprising the nucleic acid sequence of SEQ ID NO: 48;
d) a polyA sequence comprising the nucleic acid sequences of SEQ ID NO: 136;
e) at least one DNA spacer sequence comprising the nucleic acid sequences of SEQ ID NO: 137; and
f) a second AAV ITR sequence comprising the nucleic acid sequences of SEQ ID NO: 4.

Embodiment 103. The AAV transposase polynucleotide of any one of Embodiments 102A-102I, wherein the at least one promoter sequence comprises the nucleic acid sequence of SEQ ID NO: 9.

Embodiment 104. The AAV transposase polynucleotide of any one of Embodiments 102A-102I, wherein the at least one promoter sequence comprises the nucleic acid sequence of SEQ ID NO: 126.

Embodiment 105. The AAV piggyBac transposase polynucleotide of Embodiment 102A-104, wherein the at least one promoter sequence comprises the nucleic acid sequence of SEQ ID NO: 144.

Embodiment 106. A vector comprising the AAV transposase polynucleotide of any of the preceding embodiments.

Embodiment 107. The vector of any of the preceding embodiments, wherein the vector is a viral vector.

Embodiment 108. The vector of any of the preceding embodiments, wherein the viral vector is an adeno-associated virus (AAV) viral vector.

Embodiment 109. The vector of any of the preceding embodiments, wherein the AAV viral vector is an AAV1, AAV2, AAV3, AAV4, AAV5, AAV6, AAV7, AAV8, AAV9, AAV10 or AAV11 viral vector.

Embodiment 110. The vector of any of the preceding embodiments, wherein the AAV viral vector is an AAV-KP-1 or AAV-NP59 AAV viral vector, preferably wherein the AAV viral vector is an AAV-KP-1 viral vector.

Embodiment 111. A composition comprising the vector of any of embodiments 102-110.

Embodiment 112. A composition comprising the vector of any of embodiments 78-82 and the vector of any of embodiments 102-110.

Embodiment 113. A method of treating at least one metabolic liver disorder (MLD) in a subject in need thereof comprising administering to the subject at least one therapeutically effective amount of at least one polynucleotide, vector or composition of any of the preceding embodiments.

Embodiment 114. A method of treating at least one metabolic liver disorder (MLD) in a subject in need thereof comprising administering to a subject:
a) at least one therapeutically effective amount of the AAV piggyBac transposon polynucleotide of any one of the preceding embodiments, or any one of the vectors and/or compositions of the preceding embodiments that comprise an AAV piggyBac transposon polynucleotide; and
b) at least one therapeutically effective amount of the AAV piggyBac transposase polynucleotide of any of the preceding embodiments, or any one of the vectors and/or compositions of the preceding embodiments that comprise an AAV piggyBac transposase polynucleotide.

Embodiment 115. The method of embodiment 114, wherein the at least one MLD is N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome), methylmalonic acidemia (MMA), progressive familial intrahepatic cholestasis type 1 (PFIC1), progressive familial intrahepatic cholestasis type 1 (PFIC2), progressive familial intrahepatic cholestasis type 1 (PFIC3) or any combination thereof.

Embodiment 116. The method of Embodiment 115, wherein the MLD is Ornithine Transcarbamylase (OTC) Deficiency.

### Definitions

### Nucleic Acid and Polynucleotide Molecules

Nucleic acid molecules and polynucleotide molecules of the present disclosure can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

### Construction of Nucleic Acid and Polynucleotide Molecules

The nucleic acid and polynucleotide molecules of the present disclosure can be made using (a) recombinant methods, (b) synthetic techniques, (c) purification techniques, and/or (d) combinations thereof, as well-known in the art.

The nucleic acid and polynucleotide molecules can conveniently comprise nucleotide sequences in addition to a polynucleotide of the present disclosure. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art.

### Recombinant Methods for Constructing Nucleic Acid and Polynucleotide Molecules

The nucleic acid and polynucleotide molecules of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library. The isolation of RNA, and construction of cDNA and genomic libraries are well known to those of ordinary skill in the art.

### Nucleic Acid Screening and Isolation Methods

A cDNA or genomic library can be screened using a probe based upon the sequence of a polynucleotide of the disclosure. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the disclosure without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. Nos. 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference.

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The nucleic acid and polynucleotide molecules of the disclosure can also be prepared by direct chemical synthesis by known methods. Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The disclosure further provides recombinant expression cassettes comprising a nucleic acid or polynucleotide molecule of the present disclosure. A nucleic acid or polynucleotide of the present disclosure can be used to construct a recombinant expression cassette that can be introduced into at least one desired host cell. A recombinant expression cassette will typically comprise a polynucleotide of the disclosure operably linked to transcriptional initiation regulatory sequences that will direct the transcription of the polynucleotide in the intended host cell. Both heterologous and non-heterologous (i.e., endogenous) promoters can be employed to direct expression of the nucleic acids of the disclosure.

In some aspects, isolated nucleic acids that serve as promoter, enhancer, or other elements can be introduced in the appropriate position (upstream, downstream or in the intron) of a non-heterologous form of a polynucleotide of the disclosure so as to up or down regulate expression of a polynucleotide of the disclosure. For example, endogenous promoters can be altered in vivo or in vitro by mutation, deletion and/or substitution.

### Expression Vectors and Host Cells

The disclosure also relates to vectors that include isolated nucleic acid and polynucleotide molecules of the disclosure, host cells that are genetically engineered with the recombinant vectors, and the production of at least polynucleotide by recombinant techniques, as is well known in the art.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124(8):1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCE, RAD51C, GCS, MDR1, ALDH1, NKX2.2, or any combination thereof.

Those of ordinary skill in the art are knowledgeable in the numerous expression systems available for expression of a nucleic acid or polynucleotide molecule. Alternatively, nucleic acids of the disclosure can be expressed in a host cell by turning on (by manipulation) in a host cell that contains endogenous DNA encoding a nucleic acid or polynucleotide of the present disclosure. Such methods are well known in the art, e.g., as described in U.S. Pat. Nos. 5,580,734, 5,641,670, 5,733,746, and 5,733,761, entirely incorporated herein by reference

Illustrative of cell cultures useful for the production of the nucleic acid and polynucleotide molecules of the present disclosure, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a preferred aspect, the recombinant cell is a P3X63Ab8.653 or an SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof. In some aspects, a transgene sequence, or any other sequence, is said to be operatively linked to a promoter sequence when the promoter sequence controls the expression of the transgene sequence, or any other sequence. In some aspects, a transposase sequence is said to be operatively linked to a promoter sequence when the promoter sequence controls the expression of the transposase sequence.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of +2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within +2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table A.

TABLE A
Conservative Substitutions I

| | |
|---|---|
| Side chain characteristics | Amino Acid |

| | | |
|---|---|---|
| Aliphatic | Non-polar | G A P I L V F |
| | Polar - uncharged | C S T M N Q |
| | Polar - charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| | |
|---|---|
| Side Chain Characteristic | Amino Acid |

| | | |
|---|---|---|
| Non-polar | Aliphatic: | A L I V P |
| (hydrophobic) | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (b12seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

### Example 1—In Vivo Expression of Transgenes Mediated by Viral Vectors of the Present Disclosure

In the following non-limiting example, mice were treated with viral vectors of the present disclosure and expression of the transgenes contained in the viral vectors was monitored.

Newborn mice were split into four different treatment groups.

Mice in Treatment Group #1 were administered an AAV piggyBac transposon vector comprising the AAV piggyBac transposon polynucleotide described in FIG. 1 at a dosage of 3.3×10¹³ vector genomes (vg)/kg.

Mice in Treatment Group #2 were administered an AAV piggyBac transposon vector comprising the AAV piggyBac transposon polynucleotide described in FIG. 8 at a dosage of 3.3×10¹³ vg/kg.

Mice in Treatment Group #3 were administered an AAV piggyBac transposon vector comprising the AAV piggyBac transposon polynucleotide described in FIG. 1 at a dosage of 3.3×10¹³ vg/kg and an AAV transposase vector comprising an AAV transposase polynucleotide at a dosage of 1.1×10¹³ vg/kg. The AAV transposase polynucleotide comprised a transposase sequence encoding for a SPB transposase.

Mice in Treatment Group #4 were administered an AAV piggyBac transposon vector comprising the AAV piggyBac transposon polynucleotide described in FIG. 8 at a dosage of 3.3×10¹³ vg/kg and an AAV transposase vector comprising an AAV transposase polynucleotide at a dosage of 1.1×10¹³ vg/kg. The AAV transposase polynucleotide comprised a transposase sequence encoding for a SPB transposase.

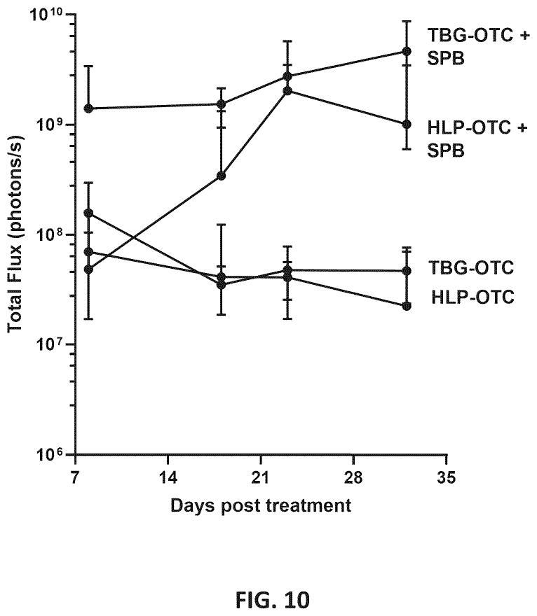

Bioluminescence (BLI) signals were then measured in the mice for 35 days following viral vector administration to measure the expression of the transgenes encoded for in the AAV piggyBac transposon polynucleotides. The results of this analysis are shown in FIG. 10. In FIG. 10, Treatment Group #1 is referred to as HLP-OTC, Treatment Group #2 is referred to as TBG-OTC, Treatment Group #3 is referred to as HLP-OTC+SPB and Treatment Group #4 is referred to as TBG-OTC+SPB.

As shown in FIG. 10, mice in Treatment Groups #3 and #4 shows increased levels of BLI over the course of the 35 days. Without wishing to be bound by theory, these results indicate that the coadministration of the AAV piggyBac transposon vector and the AAV transposase vector can lead to an integration of the transgenes of the AAV piggyBac transposon vector into the host's genome, resulting in increased and sustained expression of the transgenes. Moreover, increased expression of the transgenes was observed in Treatment Group #4, which were administered AAV piggyBac transposon vectors comprising a TBG promoter. Without wishing to be bound by theory, these results indicate that the use of a TBG promoter can provide increased transgene expression that occurs soon after administration. Such activity is particularly advantageous in a clinical setting in which early-onset patients are being treated.

### Example 2—In Vivo Expression of Transgenes Mediated by Different Concentrations of Viral Vectors of the Present Disclosure

In the following non-limiting example, mice were treated with varying concentrations of viral vectors of the present disclosure and expression of the transgenes contained in the viral vectors was monitored.

The mice in the study were administered either:
a) increasing concentrations of an AAV piggyBac transposon vector comprising the AAV piggyBac transposon polynucleotide described in FIG. 8 alone; or
b) increasing concentrations of an AAV piggyBac transposon vector comprising the AAV piggyBac transposon polynucleotide described in FIG. 8 in combination with an AAV transposase vector comprising an AAV transposase polynucleotide, wherein the AAV transposase polynucleotide comprised a transposase sequence encoding for a SPB transposase

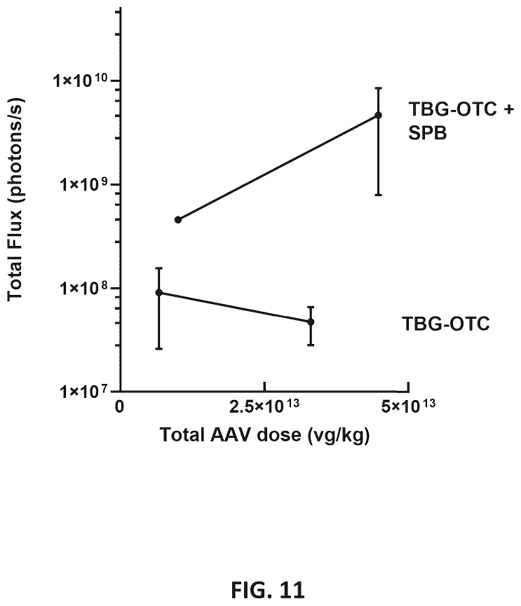

On day 21 following administration of the viral vectors, BLI was measured in the mice to measure the expression of the transgenes encoded for in the AAV piggyBac transposon polynucleotides. The results of this analysis are shown in FIG. 11. As shown in FIG. 11, higher levels of transgene expression were achieved at lower doses when both the AAV piggyBac transposon vector and the AAV transposase vector were co-administered. Without wishing to be bound by theory, these results indicate that the coadministration of the AAV piggyBac transposon vector and the AAV transposase vector can lead to an integration of the transgenes of the AAV piggyBac transposon vector into the host's genome, resulting in increased and sustained expression of the transgenes. This is particularly advantageous in a clinical setting, as it can reduce the total dose of AAV that needs to be administered to a subject, which can help to avoid the negative side effects typically associated with the administration of an AAV vector.

### Example 3—Treatment of Otc^{spf-Ash} Mice with Viral Vectors of the Present Disclosure

In the following non-limiting example, Otc^{spf-ash revl}mice were treated with viral vectors of the present disclosure.

As would be appreciated by the skilled artisan, Otc^{spf-ash} mice are a widely used model of urea cycle disorders, including OTC deficiency and chronic hyperammonemia. The mice contain a mutation (c. 386G>A; p. R129H) in the last nucleotide of exon 4 of the Otc gene, affecting the 5′ splice site and resulting in partial use of a cryptic splice site 48 bp into the adjacent intron.

Newborn Otc^{spf-ash} mice were split into two different treatment groups.

Mice in Treatment Group #1 were administered an AAV piggyBac transposon vector comprising the AAV piggyBac transposon polynucleotide described in FIG. 1 at a dosage of 3.3×10¹³ vector genomes (vg)/kg and an AAV transposase vector comprising the AAV transposase polynucleotide described in FIG. 4 at a dosage of 3.3×10¹³ vg/kg. Thus, mice in Treatment Group #1 were treated with an AAV piggyBac transposon vector and an AAV transposase vector at a dosage ratio of 1:1, with a total AAV dosage of 6.6×10¹³ vg/kg. The AAV transposase polynucleotide comprised a transgene sequence encoding for human OTC, allowing it to be distinguished from endogenous, murine OTC.

Mice in Treatment Group #2 were administered an AAV piggyBac transposon vector comprising the AAV piggyBac transposon polynucleotide described in FIG. 1 at a dosage of 3.3×10¹³ vector genomes (vg)/kg and an AAV transposase vector comprising the AAV transposase polynucleotide described in FIG. 4 at a dosage of 1.7×10¹³ vg/kg. Thus, mice in Treatment Group #2 were treated with an AAV piggyBac transposon vector and an AAV transposase vector at a dosage ratio of 2:1, with a total AAV dosage of 5×10¹³ vg/kg. The AAV transposase polynucleotide comprised a transgene sequence encoding for human OTC, allowing it to be distinguished from endogenous, murine OTC.

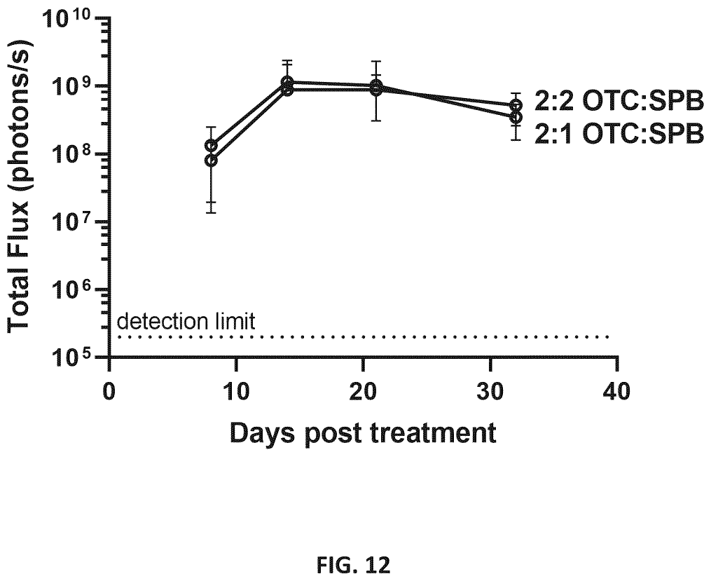

Following administration of the viral vectors, BLI was measured in the mice to measure the expression of the transgenes encoded for in the AAV piggyBac transposon polynucleotide. The results of this analysis are shown in FIG. 12. As shown in FIG. 12, high levels of transgene expression was measured in both of the treatment groups.

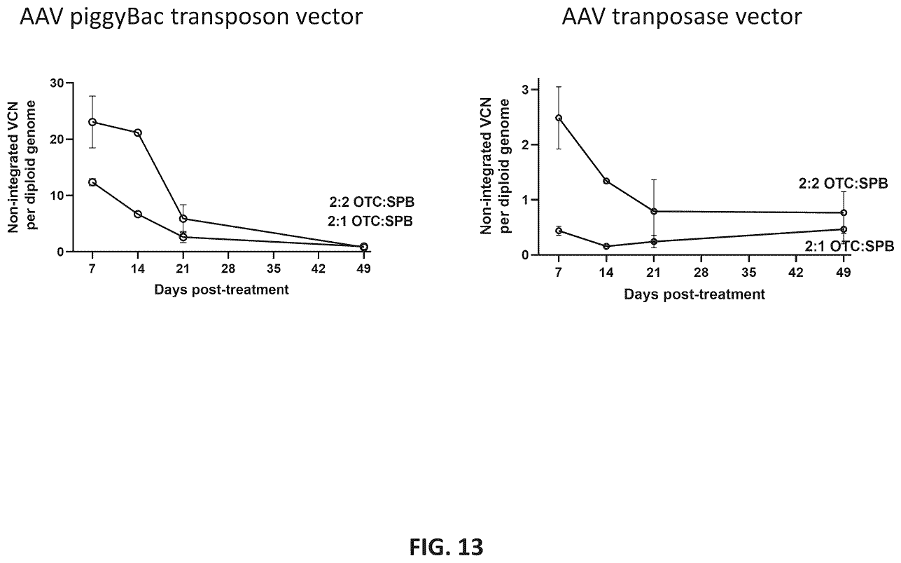

Following administration of the viral vectors, the amount of non-integrated vector copy number per diploid genome for both the AAV piggyBAC transposon vector and the AAV transposase vector were measured. The results of this analysis are shown in FIG. 13. As shown in FIG. 13, the amount of non-integrated AAV transposase vector decreased as the mice aged. Moreover, the amount of non-integrated AAV transposase vector was lower as compared to the amount of non-integrated AAV piggyBac transposon vector, even at day 7.

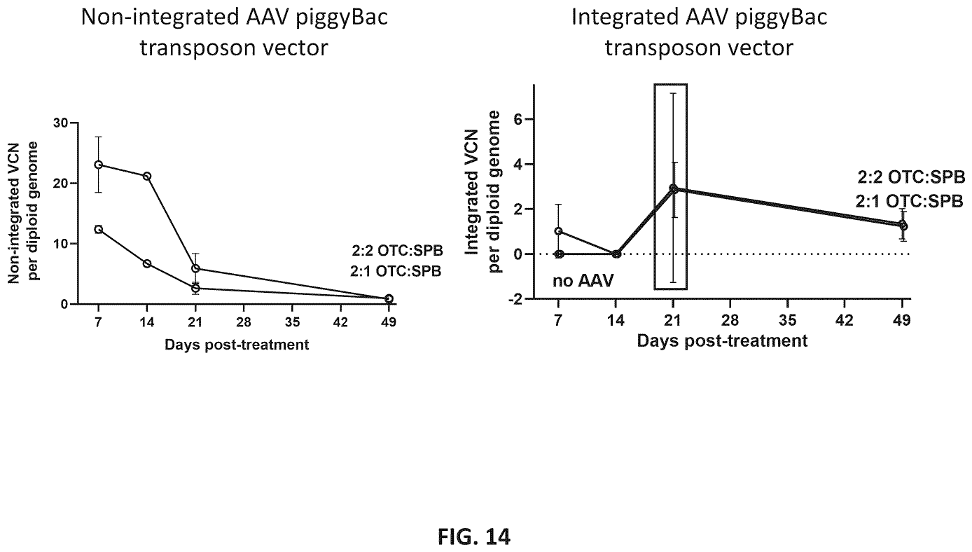

Following administration of the viral vectors, the amount of non-integrated AAV piggyBac transposon vector copy number per diploid genome and the amount of integrated AAV piggyBac transposon vector copy number were measured. The results of this analysis are shown in FIG. 14. As shown in FIG. 14, there was detection of integrated AAV piggbyBac transposon vector at 21 days post treatment. Moreover, the integration was more consistent in Treatment Group #1 (2:1 OTC:SPB) as compared to Treatment Group #2 (2:2 OTC:SPB). Without wishing to be bound by theory, the results in FIG. 14, specifically the integration of the transposon vector, indicate successful transposition of the transposon in vivo.

The number of integrated sites was also assayed by LM-PCR at days 21 and 43. Briefly, two ug of genomic DNA was isolated from mice liver tissue and sheared randomly by sonication. Unique molecular identifiers (UMI) were ligated onto the resulting ends. Two rounds of PCR amplification were performed. The final PCR product was Illumina paired-end sequenced. The integrated sites were determined by double-site break point confirmation. The results of this PCR analysis are shown in Tables 1 and 2. Without wishing to be bound by theory, the results presented in Tables 1 and 2, indicate successful transposition and integration of the transposon in vivo.

TABLE 1

| | UMI reads | Integrated site # |
|---|---|---|
| OTC + SPB (2:2) | 14,797,590 | 317 |
| OTC + SPB (2:1) | 13,119,967 | 86 |
| OTC + SPB (2:1) | 17,118,928 | 48 |
| OTC + SPB (2:1) | 13,692,000 | 68 |
| OTC + SPB (2:2) | 13,908,440 | 65 |

TABLE 2

| | UMI reads | Integrated site # |
|---|---|---|
| Control | 3,424,155 | 0 |
| OTC + SPB (2:2) | 9,951,278 | 21 |
| OTC + SPB (2:2) | 11,682,573 | 50 |
| OTC + SPB (2:2) | 13,347,536 | 14 |
| OTC + SPB (2:2) | 9,450,364 | 109 |
| OTC + SPB (2:2) | 9,162,781 | 20 |
| OTC + SPB (2:2) | 9,565,134 | 23 |
| OTC + SPB (2:1) | 12,425,240 | 73 |
| OTC + SPB (2:1) | 9,989,032 | 10 |
| OTC + SPB (2:1) | 12,831,700 | 6 |
| OTC + SPB (2:1) | 8,507,944 | 8 |
| OTC + SPB (2:1) | 3,828,516 | 12 |

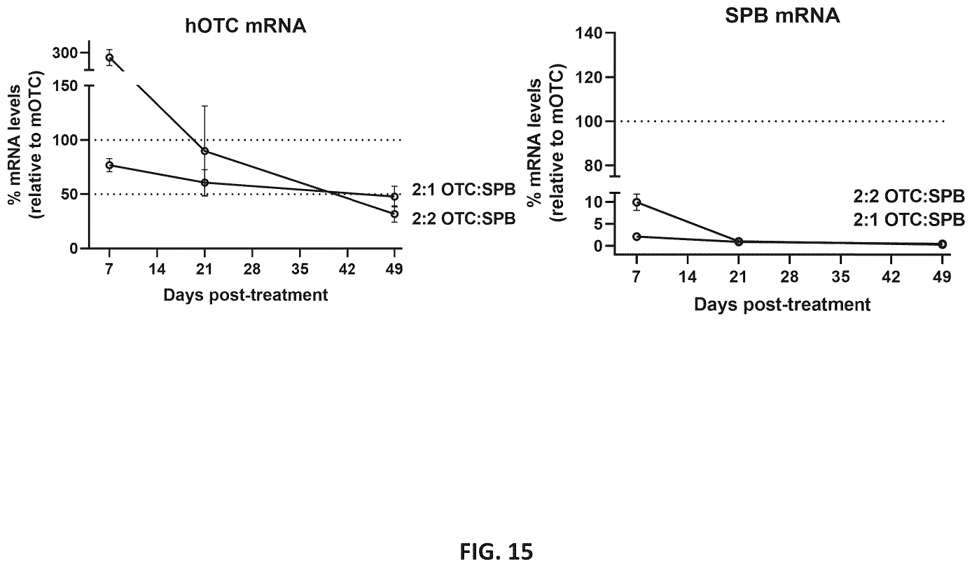

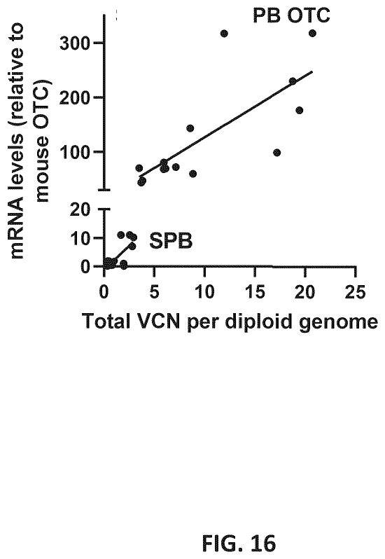

Following administration of the viral vectors, the amount of human OTC mRNA and SPB mRNA relative to the levels of murine OTC mRNA were measured in the mice. The results of this analysis are shown in FIG. 15. As shown in FIG. 15, mice treated with the viral vectors express significant amounts of human OTC mRNA. Moreover, the level of SPB mRNA decrease with age. Without wishing to be bound by theory, this decrease in SPB mRNA can be advantageous in a clinical setting as to avoid off-target transposition effects following initial treatment. A correlation analysis between human OTC mRNA and SPB mRNA vs total vector copy number per diploid genome was also performed. The results of this analysis are shown in FIG. 16. As shown in FIG. 16, mRNA levels of human OTC and SPB correlated with the corresponding vector copy numbers.

At day 21 post treatment, liver samples were collected from the mice and GFP expression was analyzed. Liver cells in the samples collected from both Treatment Group #1 and Treatment Group #2 displayed robust GFP expression.

### Example 4—Treatment of an Inducible Hyperammonemic Mouse Model with Viral Vectors of the Present Disclosure

In the following non-limiting example, Otc^{spf-ash} mice were treated with viral vectors of the present disclosure and shRNA was used to create an induced hyperammonemic morbidity model.

Newborn Otc^{spf-ash} mice were split into two different treatment groups.

Mice in Treatment Group #1 were administered an AAV piggyBac transposon vector comprising the AAV piggyBac transposon polynucleotide described in FIG. 1 at a dosage of 3.3×10¹³ vector genomes (vg)/kg and an AAV transposase vector comprising the AAV transposase polynucleotide described in FIG. 4 at a dosage of 3.3×10¹³ vg/kg. Thus, mice in Treatment Group #1 were treated with an AAV piggyBac transposon vector and an AAV transposase vector at a dosage ratio of 1:1, with a total AAV dosage of 6.6×10¹³ vg/kg. The AAV transposase polynucleotide comprised a transgene sequence encoding for human OTC, allowing it to be distinguished from endogenous, murine OTC.

Mice in Treatment Group #2 were administered an AAV piggyBac transposon vector comprising the AAV piggyBac transposon polynucleotide described in FIG. 1 at a dosage of 3.3×10¹³ vector genomes (vg)/kg and an AAV transposase vector comprising the AAV transposase polynucleotide described in FIG. 4 at a dosage of 1.7×10¹³ vg/kg. Thus, mice in Treatment Group #2 were treated with an AAV piggyBac transposon vector and an AAV transposase vector at a dosage ratio of 2:1, with a total AAV dosage of 5×10¹³ vg/kg. The AAV transposase polynucleotide comprised a transgene sequence encoding for human OTC, allowing it to be distinguished from endogenous, murine OTC.

At 38 days post treatment, a subset in each treatment group were either:
a) left further untreated
b) administered a dosage of shRNA targeting mouse OTC.

As a control and for comparison, Otc^{spf-ash} mice of similar age that were not treated with the viral vectors were also administered the dosage of shRNA targeting mouse OTC.

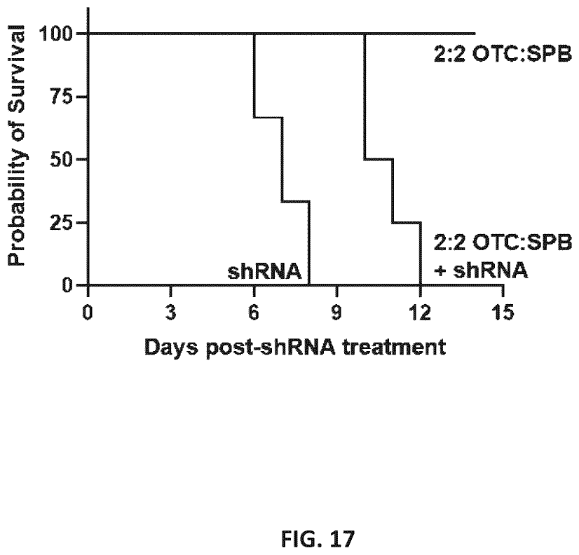

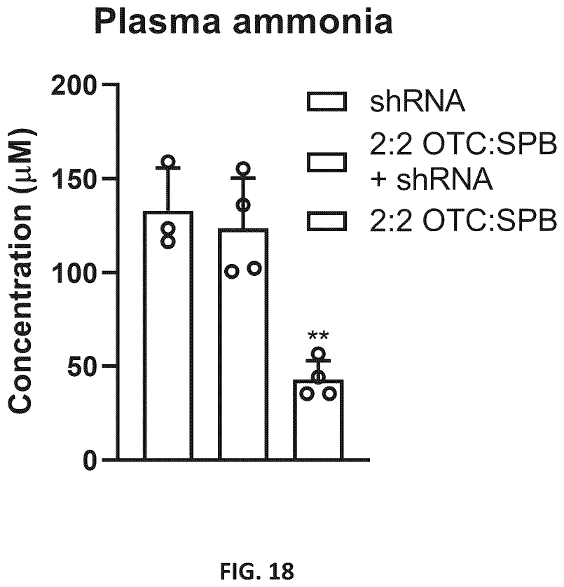

FIG. 17 shows the probability of survival of mice in Treatment Group #1 that were either left further untreated (2:2 OTC:SPB) or that were further administered a dosage of shRNA targeting mouse OTC (2:2: OTC:SPB+shRNA). FIG. 17 also shows the probability of survival of Otc^{spf-ash} mice of similar age that were not treated with the viral vectors and that were also administered the dosage of shRNA targeting mouse OTC. FIG. 18 shows the concentration of ammonia in the plasma of the aforementioned groups of mice. As shown in FIG. 17 and FIG. 18, the deleterious effects of administering the shRNA are delayed in mice that were treated with the viral vectors.

### Example 5—In Vivo Expression of Transgenes Operably Linked to Different Promoter Sequences in AAV piggyBac Transposon Vectors of the Present Disclosure

In the following non-limiting example, mice were treated with viral vectors of the present disclosure comprising transgenes operably linked to either an HLP promoter, an LP1 promoter or a TBG promoter sequence. Expression of the transgenes contained in the viral vectors was monitored to determine the efficiency with which each promoter was able to drive transgene expression in vivo, specifically within the liver.

Newborn wildtype mice and adult wildtype mice and newborn Otc^{spf-ash} mice were split into 12 different treatment groups.

Treatment Groups #1-#6 comprised newborn wildtype mice.

Mice in Treatment Group #1 were administered an AAV piggyBac transposon vector comprising the AAV piggyBac transposon polynucleotide described in FIG. 1 at a dosage of 5×10¹³ vg/kg.

Mice in Treatment Group #2 were administered an AAV piggyBac transposon vector comprising the AAV piggyBac transposon polynucleotide described in FIG. 1 at a dosage of 3.3×10¹³ vg/kg and an AAV transposase vector comprising an AAV transposase polynucleotide at a dosage of 1.7×10¹³ vg/kg. The AAV transposase polynucleotide comprised a transposase sequence encoding for a SPB transposase operably linked to an HLP promoter.

Mice in Treatment Group #3 were administered an AAV piggyBac transposon vector comprising the AAV piggyBac transposon polynucleotide described in FIG. 8 at a dosage of 5×10¹³ vg/kg.

Mice in Treatment Group #4 were administered an AAV piggyBac transposon vector comprising the AAV piggyBac transposon polynucleotide described in FIG. 8 at a dosage of 3.3×10¹³ vg/kg and an AAV transposase vector comprising an AAV transposase polynucleotide at a dosage of 1.7×10¹³ vg/kg. The AAV transposase polynucleotide comprised a transposase sequence encoding for a SPB transposase operably linked to an HLP promoter.

Mice in Treatment Group #5 were administered an AAV piggyBac transposon vector comprising the AAV piggyBac transposon polynucleotide described in FIG. 9 at a dosage of 5×10¹³ vg/kg.

Mice in Treatment Group #6 were administered an AAV piggyBac transposon vector comprising the AAV piggyBac transposon polynucleotide described in FIG. 9 at a dosage of 3.3×10¹³ vg/kg and an AAV transposase vector comprising an AAV transposase polynucleotide at a dosage of 1.7×10¹³ vg/kg. The AAV transposase polynucleotide comprised a transposase sequence encoding for a SPB transposase operably linked to an HLP promoter.

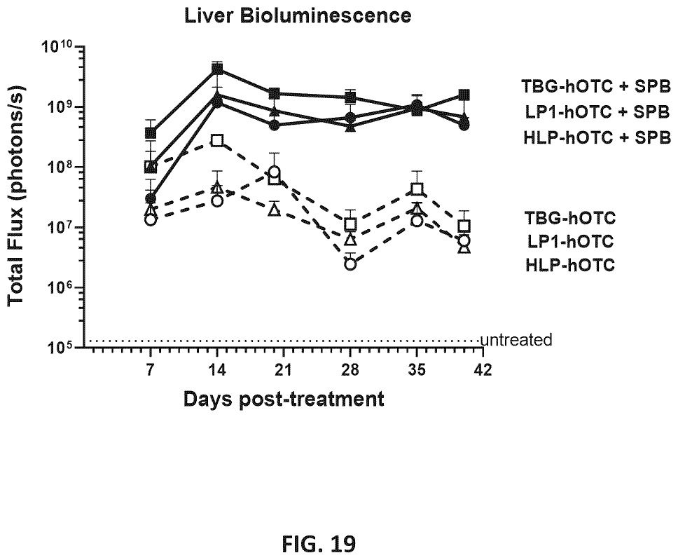

Liver bioluminescence (BLI) signals were then measured in the mice in Treatment Groups #1-#6 for 42 days following viral vector administration to measure the expression of the transgenes encoded for in the AAV piggyBac transposon polynucleotides. The results of this analysis are shown in FIG. 19. In FIG. 19, Treatment Group #1 is referred to as HLP-OTC, Treatment Group #2 is referred to as HLP-OTC+SPB, Treatment Group #3 is referred to as TBG-OTC, Treatment Group #4 is referred to as TBG-OTC+SPB, Treatment Group #5 is referred to as LP1-OTC+SPB and Treatment Group #6 is referred to as LP1-OTC+SPB. As shown in FIG. 19, the TBG promoter drove transgene expression most efficiently, followed by the LP1 promoter and then the HLP promoter. Increased and sustained expression was observed in treatment Groups #2, #4 and #6. Without wishing to be bound by theory, these results indicate that the coadministration of the AAV piggyBac transposon vector and the AAV transposase vector can lead to an integration of the transgenes of the AAV piggyBac transposon vector into the host's genome, resulting in increased and sustained expression of the transgenes. The integration of the transposon vector observed in FIG. 19 indicates successful transposition of the transposon in vivo.

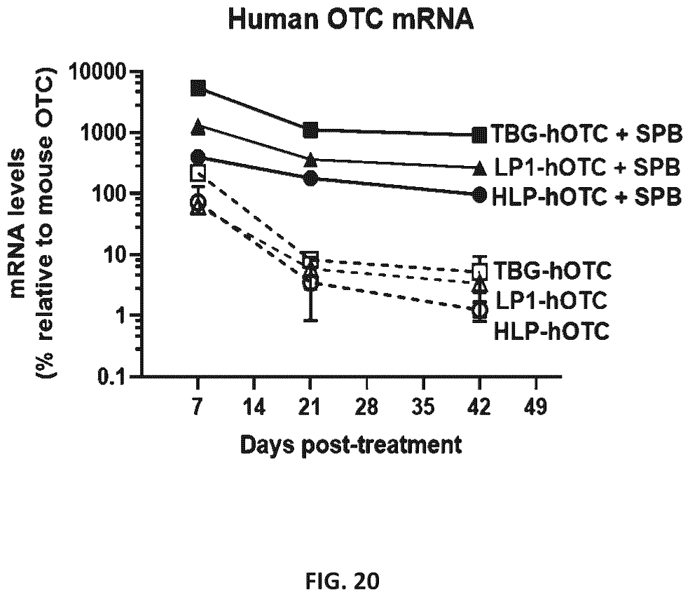

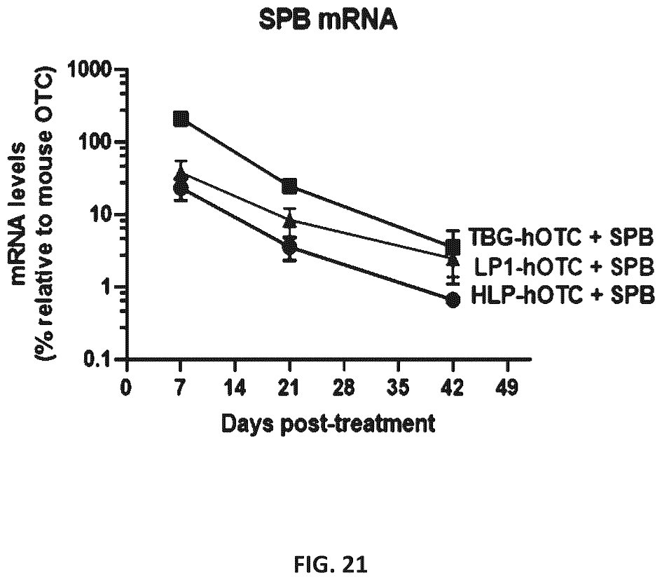

The amount of human OTC mRNA and SPB mRNA relative to the levels of murine OTC mRNA were also measured in the mice of Treatment Groups #1-#6. The results of this analysis are shown in FIG. 20 (human OTC mRNA) and FIG. 21 (SPB mRNA). Similar to the results shown in FIG. 19, the results in FIG. 20 and FIG. 21 show that the TBG promoter yielded the highest levels of transgene mRNA, followed by the LP1 promoter, and then the HLP promoter.

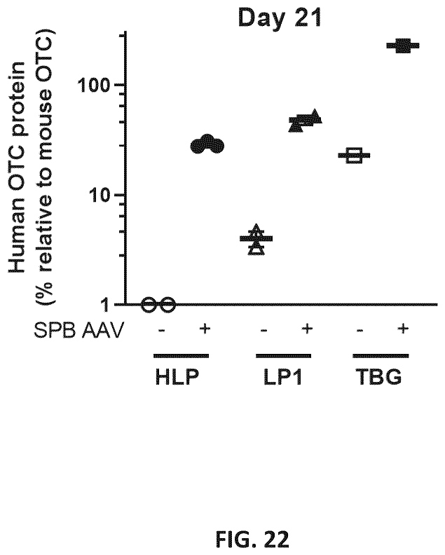

The amount of human OTC protein relative to the amount of mouse OTC protein was also measured on Day 21 following viral vector administration. The results of this analysis are shown in FIG. 22. Similar to the results shown in FIGS. 19-21, the results shown in FIG. 22 show that the TBG promoter yielded the highest levels of human OTC protein, followed by the LP1 promoter, and then the HLP promoter.

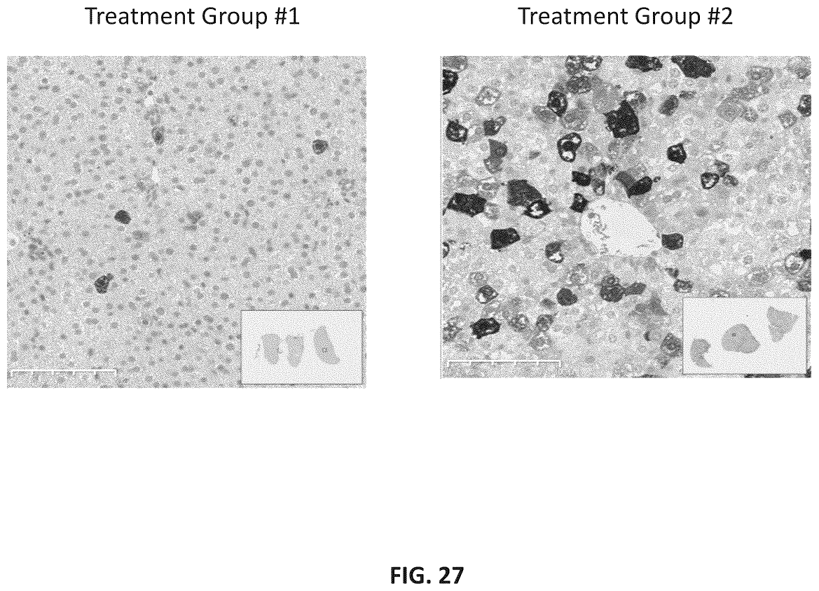

Additionally, hepatocytes from mice in Treatment Group #1 and Treatment #2 were also analyzed by immunohistochemistry and stained for GFP. Briefly, liver tissue was collected at day 21, fixed in 10% neutral-buffered formalin and paraffin-embedded prior to staining. The results of the immunohistochemistry results are shown in FIG. 27, which shows that higher levels of GFP were observed in Treatment Group #2. Without wishing to be bound by theory, these results indicate that the coadministration of the AAV piggyBac transposon vector and the AAV transposase vector can lead to an integration of the transgenes of the AAV piggyBac transposon vector into the host's genome, resulting in increased and sustained expression of the transgenes.

Additionally, the number of integrated sites in the genome of the treated mice were assayed by LM-PCR. Briefly, two ug of genomic DNA was isolated from mice liver tissue and sheared randomly by sonication. Unique molecular identifiers (UMI) were ligated onto the resulting ends. Two rounds of PCR amplification were performed. The final PCR product was Illumina paired-end sequenced. Total unique integration sites were determined by single side break point with 2 or more UMI. The results of this analysis are shown in Table 3.

TABLE 3

| | | | Total Unique |
|---|---|---|---|
| | | | Integration Sites |
| | Input DNA | | by single-side |
| | per reaction | | break point |
| Sample | (μg) | Total Reads | (UMI >= 2) |
| AAV-HLP-OTC + | 2 | 24,683,166 | 36,868 |
| AAV-SPB | | | |
| AAV-HLP-OTC + | 2 | 16,599,187 | 26,757 |
| AAV-SPB | | | |
| AAV-LP1-OTC only | 2 | 25,092,023 | 24 |
| AAV-LP1-OTC + | 2 | 21,839,543 | 39,098 |
| AAV-SPB | | | |

Treatment Groups #7-#12 comprised adult wildtype mice.

Mice in Treatment Group #7 were administered an AAV piggyBac transposon vector comprising the AAV piggyBac transposon polynucleotide described in FIG. 1 at a dosage of 2×10¹³ vg/kg.

Mice in Treatment Group #8 were administered an AAV piggyBac transposon vector comprising the AAV piggyBac transposon polynucleotide described in FIG. 1 at a dosage of 1.3×10¹³ vg/kg and an AAV transposase vector comprising an AAV transposase polynucleotide at a dosage of 0.7×10¹³ vg/kg. The AAV transposase polynucleotide comprised a transposase sequence encoding for a SPB transposase operably linked to an HLP promoter.

Mice in Treatment Group #9 were administered an AAV piggyBac transposon vector comprising the AAV piggyBac transposon polynucleotide described in FIG. 8 at a dosage of 2×10¹³ vg/kg.

Mice in Treatment Group #10 were administered an AAV piggyBac transposon vector comprising the AAV piggyBac transposon polynucleotide described in FIG. 8 at a dosage of 1.3×10¹³ vg/kg and an AAV transposase vector comprising an AAV transposase polynucleotide at a dosage of 0.7×10¹³ vg/kg. The AAV transposase polynucleotide comprised a transposase sequence encoding for a SPB transposase operably linked to an HLP promoter.

Mice in Treatment Group #11 were administered an AAV piggyBac transposon vector comprising the AAV piggyBac transposon polynucleotide described in FIG. 9 at a dosage of 2×10¹³ vg/kg.

Mice in Treatment Group #12 were administered an AAV piggyBac transposon vector comprising the AAV piggyBac transposon polynucleotide described in FIG. 9 at a dosage of 1.3×10¹³ vg/kg and an AAV transposase vector comprising an AAV transposase polynucleotide at a dosage of 0.7×10¹³ vg/kg. The AAV transposase polynucleotide comprised a transposase sequence encoding for a SPB transposase operably linked to an HLP promoter.

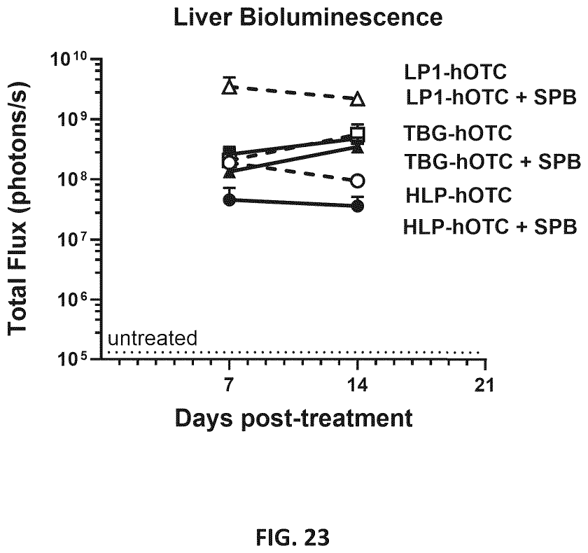

Liver bioluminescence (BLI) signals were then measured in the mice in Treatment Groups #7-#12 at 7 and 14 days following viral vector administration to measure the expression of the transgenes encoded for in the AAV piggyBac transposon polynucleotides. The results of this analysis are shown in FIG. 23. In FIG. 23, Treatment Group #7 is referred to as HLP-OTC, Treatment Group #8 is referred to as HLP-OTC+SPB, Treatment Group #9 is referred to as TBG-OTC, Treatment Group #10 is referred to as TBG-OTC+SPB, Treatment Group #11 is referred to as LP1-OTC+SPB and Treatment Group #12 is referred to as LP1-OTC+SPB. As shown in FIG. 23, a similar strength of transgene expression was observed for each of the promoters.

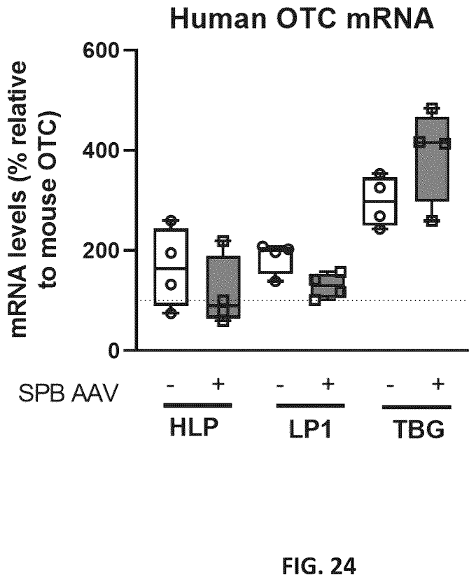

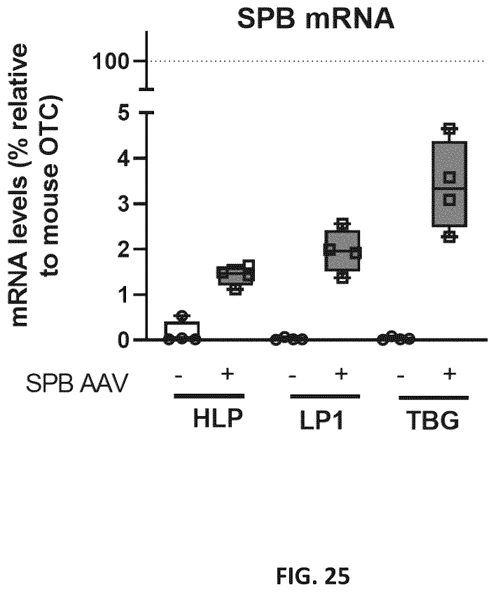

The amount of human OTC mRNA and SPB mRNA relative to the levels of murine OTC mRNA were also measured at 14 days following viral vector administration in the mice of Treatment Groups #7-#12. The results of this analysis are shown in FIG. 24 (human OTC mRNA) and FIG. 25 (SPB mRNA). As shown in FIG. 24 and FIG. 25, a similar level of human OTC was observed for the HLP and LP1 promoter, with the strongest expression observed with the TBG promoter.

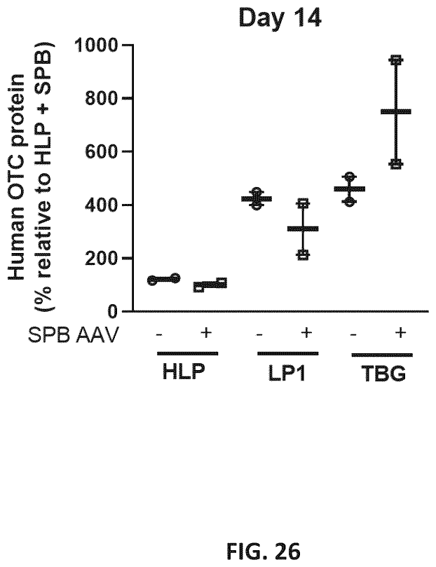

The amount of human OTC protein relative to the amount of mouse OTC protein was also measured on Day 14 following viral vector administration. The results of this analysis are shown in FIG. 26. Similar to the results shown in FIGS. 19-21, the results shown in FIG. 22 show that the TBG promoter yielded the highest levels of human OTC protein, followed by the LP1 promoter, and then the HLP promoter.

### EQUIVALENTS

The foregoing description has been presented only for the purposes of illustration and is not intended to limit the disclosure to the precise form disclosed. The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

1. An adeno-associated virus (AAV) piggyBac transposon polynucleotide comprising in the 5′ to 3′ direction:
(i)
a) a first AAV ITR sequence comprising the nucleic acid sequence of SEQ ID NO: 3;
b) a first piggyBac ITR sequence comprising the nucleic acid sequence of SEQ ID NO: 125;
c) a first insulator sequence comprising the nucleic acid sequence of SEQ ID NO: 7;
d) at least one promoter sequence comprising the nucleic acid sequence of SEQ ID NO: 126;
e) at least one transgene sequence comprising the nucleic acid sequence of SEQ ID NO: 22;
f) a polyA sequence comprising the nucleic acid sequence of SEQ ID NO: 97;
g) a second insulator sequence comprising the nucleic acid sequence of SEQ ID NO: 8;
h) a second piggyBac ITR sequence comprising the nucleic acid sequence of SEQ ID NO: 96;
i) at least one DNA spacer sequence comprising the nucleic acid sequence of SEQ ID NO: 129; and
j) a second AAV ITR sequence comprising the nucleic acid sequence of SEQ ID NO: 4;
(ii)
a) a first AAV ITR sequence comprising the nucleic acid sequence of SEQ ID NO: 3;
b) a first piggyBac ITR sequence comprising the nucleic acid sequence of SEQ ID NO: 125;
c) a first insulator sequence comprising the nucleic acid sequence of SEQ ID NO: 7;
d) at least one promoter sequence comprising the nucleic acid sequence of SEQ ID NO: 132;
e) at least one transgene sequence comprising the nucleic acid sequence of SEQ ID NO: 22;
f) a poly A sequence comprising the nucleic acid sequence of SEQ ID NO: 97;
g) a second insulator sequence comprising the nucleic acid sequence of SEQ ID NO: 8;
h) a second piggyBac ITR sequence comprising the nucleic acid sequence of SEQ ID NO: 96;
i) at least one DNA spacer sequence comprising the nucleic acid sequence of SEQ ID NO: 130; and
j) a second AAV ITR sequence comprising the nucleic acid sequence of SEQ ID NO: 2; or
iii)
a) a first AAV ITR sequence comprising the nucleic acid sequence of SEQ ID NO: 3;
b) a first piggyBac ITR sequence comprising the nucleic acid sequence of SEQ ID NO: 125;
c) a first insulator sequence comprising the nucleic acid sequence of SEQ ID NO: 7;
d) at least one promoter sequence comprising the nucleic acid sequence of SEQ ID NO: 13;
e) at least one transgene sequence comprising the nucleic acid sequence of SEQ ID NO: 22;
f) a polyA sequence comprising the nucleic acid sequence of SEQ ID NO: 97;
g) a second insulator sequence comprising the nucleic acid sequence of SEQ ID NO: 8;
h) a second piggyBac ITR sequence comprising the nucleic acid sequence of SEQ ID NO: 96;
i) at least one DNA spacer sequence comprising the nucleic acid sequence of SEQ ID NO: 131; and
j) a second AAV ITR sequence comprising the nucleic acid sequence of SEQ ID NO: 4.

2. The AAV piggyBac transposon polynucleotide of claim 1, wherein the AAV piggyBac transposon polynucleotide comprises the nucleic acid sequence of (i) SEQ ID NO: 138; (ii) SEQ ID NO: 139 or (iii) SEQ ID NO: 140.

3.-6. (canceled)

7. A vector comprising the AAV piggyBac transposon polynucleotide of claim 1.

8. The vector of claim 7, wherein the vector is an AAV viral vector.

9. The vector of claim 8, wherein the AAV viral vector is an AAVl, AAV2, AAV3, AAV4, AAV5, AAV6, AAV7, AAV8, AAV9, AAVl0 or AAV11 viral vector.

10. The vector of claim 8, wherein the AAV viral vector is an AAV-KP-1 or AAV-NP59 viral vector.

11. A composition comprising the vector of claim 7.

12. An AAV transposase polynucleotide comprising in the 5′ to 3′ direction:
a) a first AAV ITR sequence comprising the nucleic acid sequence of SEQ ID NO: 127;
b) at least one promoter sequence at least one promoter sequence comprising the nucleic acid sequence of SEQ ID NO: 126;
c) at least one transposase sequence comprising the nucleic acid sequence of SEQ ID NO: 48;
d) a polyA sequence comprising the nucleic acid sequences of SEQ ID NO: 136;
e) at least one DNA spacer sequence comprising the nucleic acid sequences of SEQ ID NO: 137; and
f) a second AAV ITR sequence comprising the nucleic acid sequences of SEQ ID NO: 4.

13. The AAV transposase polynucleotide of claim 12, wherein the AAV transposase polynucleotide comprises the nucleic acid sequence of SEQ ID NO: 144.

14. A vector comprising the AAV transposase polynucleotide of claim 12.

15. The vector of claim 14, wherein the vector is an AAV viral vector.

16. The vector of claim 15, wherein the AAV viral vector is an AAV1, AAV2, AAV3, AAV4, AAV5, AAV6, AAV7, AAV8, AAV9, AAV10 or AAV11 viral vector.

17. The vector of claim 15, wherein the AAV viral vector is an AAV-KP-1 or AAV-NP59 viral vector.

18. A composition comprising the vector of claim 14.

19. A method of treating at least one metabolic liver disorder (MLD) in a subject in need thereof comprising administering to the subject at least one therapeutically effective dose of the polynucleotide of claim 1.

20. A method of treating at least one MLD in a subject in need thereof, the method comprising administering to the subject:
a) an adeno-associated virus (AAV) piggyBac transposon polynucleotide comprising in the 5′ to 3′ direction:
(i)
a) a first AAV ITR sequence comprising the nucleic acid sequence of SEQ ID NO: 3;
b) a first piggy Bac ITR sequence comprising the nucleic acid sequence of SEQ ID NO: 125;
c) a first insulator sequence comprising the nucleic acid sequence of SEQ ID NO: 7;
d) at least one promoter sequence comprising the nucleic acid sequence of SEQ ID NO: 126;
e) at least one transgene sequence comprising the nucleic acid sequence of SEQ ID NO: 22;
f) a polyA sequence comprising the nucleic acid sequence of SEQ ID NO: 97;
g) a second insulator sequence comprising the nucleic acid sequence of SEQ ID NO: 8;
h) a second piggyBac ITR sequence comprising the nucleic acid sequence of SEQ ID NO: 96;
i) at least one DNA spacer sequence comprising the nucleic acid sequence of SEQ ID NO: 129; and
j) a second AAV ITR sequence comprising the nucleic acid sequence of SEQ ID NO: 4;
(ii)
a) a first AAV ITR sequence comprising the nucleic acid sequence of SEQ ID NO: 3;
b) a first piggyBac ITR sequence comprising the nucleic acid sequence of SEQ ID NO: 125;
c) a first insulator sequence comprising the nucleic acid sequence of SEQ ID NO: 7;
d) at least one promoter sequence comprising the nucleic acid sequence of SEQ ID NO: 132;
e) at least one transgene sequence comprising the nucleic acid sequence of SEQ ID NO: 22;
f) a poly A sequence comprising the nucleic acid sequence of SEQ ID NO: 97;
g) a second insulator sequence comprising the nucleic acid sequence of SEQ ID NO: 8;
h) a second piggyBac ITR sequence comprising the nucleic acid sequence of SEQ ID NO: 96;
i) at least one DNA spacer sequence comprising the nucleic acid sequence of SEQ ID NO: 130; and
j) a second AAV ITR sequence comprising the nucleic acid sequence of SEQ ID NO: 2; or
iii)
a) a first AAV ITR sequence comprising the nucleic acid sequence of SEQ ID NO: 3;
b) a first piggyBac ITR sequence comprising the nucleic acid sequence of SEQ ID NO: 125;
c) a first insulator sequence comprising the nucleic acid sequence of SEQ ID NO: 7;
d) at least one promoter sequence comprising the nucleic acid sequence of SEQ ID NO: 13;
e) at least one transgene sequence comprising the nucleic acid sequence of SEQ ID NO: 22;
f) a polyA sequence comprising the nucleic acid sequence of SEQ ID NO: 97;
g) a second insulator sequence comprising the nucleic acid sequence of SEQ ID NO: 8;
h) a second piggyBac ITR sequence comprising the nucleic acid sequence of SEQ ID NO: 96;
i) at least one DNA spacer sequence comprising the nucleic acid sequence of SEQ ID NO: 131; and
j) a second AAV ITR sequence comprising the nucleic acid sequence of SEQ ID NO: 4; and
b) an AAV transposase polynucleotide comprising in the 5′ to 3′ direction:
a) a first AAV ITR sequence comprising the nucleic acid sequence of SEQ ID NO: 127;
b) at least one promoter sequence at least one promoter sequence comprising the nucleic acid sequence of SEQ ID NO: 126;
c) at least one transposase sequence comprising the nucleic acid sequence of SEQ ID NO: 48;
d) a polyA sequence comprising the nucleic acid sequences of SEQ ID NO: 136;
e) at least one DNA spacer sequence comprising the nucleic acid sequences of SEQ ID NO: 137; and
f) a second AAV ITR sequence comprising the nucleic acid sequences of SEQ ID NO: 4.

21. (canceled)

22. The composition of claim 18 further comprising an adeno-associated virus (AAV) piggyBac transposon polynucleotide comprising in the 5′ to 3′ direction:
(i)
a) a first AAV ITR sequence comprising the nucleic acid sequence of SEQ ID NO: 3;
b) a first piggyBac ITR sequence comprising the nucleic acid sequence of SEQ ID NO: 125;
c) a first insulator sequence comprising the nucleic acid sequence of SEQ ID NO: 7;
d) at least one promoter sequence comprising the nucleic acid sequence of SEQ ID NO: 126;
e) at least one transgene sequence comprising the nucleic acid sequence of SEQ ID NO: 22;
f) a poly A sequence comprising the nucleic acid sequence of SEQ ID NO: 97;
g) a second insulator sequence comprising the nucleic acid sequence of SEQ ID NO: 8;
h) a second piggyBac ITR sequence comprising the nucleic acid sequence of SEQ ID NO: 96;
i) at least one DNA spacer sequence comprising the nucleic acid sequence of SEQ ID NO: 129; and
j) a second AAV ITR sequence comprising the nucleic acid sequence of SEQ ID NO: 4;
(ii)
a) a first AAV ITR sequence comprising the nucleic acid sequence of SEQ ID NO: 3;
b) a first piggyBac ITR sequence comprising the nucleic acid sequence of SEQ ID NO: 125;
c) a first insulator sequence comprising the nucleic acid sequence of SEQ ID NO: 7;
d) at least one promoter sequence comprising the nucleic acid sequence of SEQ ID NO: 132;
e) at least one transgene sequence comprising the nucleic acid sequence of SEQ ID NO: 22;
f) a poly A sequence comprising the nucleic acid sequence of SEQ ID NO: 97;
g) a second insulator sequence comprising the nucleic acid sequence of SEQ ID NO: 8;
h) a second piggyBac ITR sequence comprising the nucleic acid sequence of SEQ ID NO: 96;
i) at least one DNA spacer sequence comprising the nucleic acid sequence of SEQ ID NO: 130; and
j) a second AAV ITR sequence comprising the nucleic acid sequence of SEQ ID NO: 2; or
iii)
a) a first AAV ITR sequence comprising the nucleic acid sequence of SEQ ID NO: 3;
b) a first piggyBac ITR sequence comprising the nucleic acid sequence of SEQ ID NO: 125;
c) a first insulator sequence comprising the nucleic acid sequence of SEQ ID NO: 7;
d) at least one promoter sequence comprising the nucleic acid sequence of SEQ ID NO: 13;
e) at least one transgene sequence comprising the nucleic acid sequence of SEQ ID NO: 22;
f) a poly A sequence comprising the nucleic acid sequence of SEQ ID NO: 97;
g) a second insulator sequence comprising the nucleic acid sequence of SEQ ID NO: 8;
h) a second piggyBac ITR sequence comprising the nucleic acid sequence of SEQ ID NO: 96;
i) at least one DNA spacer sequence comprising the nucleic acid sequence of SEQ ID NO: 131; and
j) a second AAV ITR sequence comprising the nucleic acid sequence of SEQ ID NO:4.

23. The method of claim 19, wherein the at least one MLD is N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome), methylmalonic acidemia (MMA), progressive familial intrahepatic cholestasis type I (PFICI), progressive familial intrahepatic cholestasis type I (PFIC2), progressive familial intrahepatic cholestasis type I (PFIC3) or any combination thereof.

24. The method of claim 23, wherein the MLD is OTC deficiency.


SEQUENCE LISTING (146 sequences)

SEQ ID NO: 1 - DNA, 178 bp, synthetic construct
  misc_feature 1..178: note = Synthetic Construct
  source 1..178: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgct

SEQ ID NO: 2 - DNA, 178 bp, synthetic construct
  misc_feature 1..178: note = Synthetic Nucleic Acid
  source 1..178: mol_type = other DNA
agcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 3 - DNA, 130 bp, synthetic construct
  misc_feature 1..130: note = Synthetic Construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 4 - DNA, 141 bp, synthetic construct
  misc_feature 1..141: note = Synthetic Nucleic Acid
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 5 - DNA, 303 bp, synthetic construct
  misc_feature 1..303: note = Synthetic Nucleic Acid
  source 1..303: mol_type = other DNA
tataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctaggg

SEQ ID NO: 6 - DNA, 238 bp, synthetic construct
  misc_feature 1..238: note = Synthetic Nucleic Acid
  source 1..238: mol_type = other DNA
ccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaactttta

SEQ ID NO: 7 - DNA, 232 bp, synthetic construct
  misc_feature 1..232: note = Synthetic Nucleic Acid
  source 1..232: mol_type = other DNA
gagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaa

SEQ ID NO: 8 - DNA, 232 bp, synthetic construct
  misc_feature 1..232: note = Synthetic Nucleic Acid
  source 1..232: mol_type = other DNA
ttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctc

SEQ ID NO: 9 - DNA, 252 bp, synthetic construct
  misc_feature 1..252: note = Synthetic Nucleic Acid
  source 1..252: mol_type = other DNA
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 10 - DNA, 449 bp, synthetic construct
  misc_feature 1..449: note = Synthetic Nucleic Acid
  source 1..449: mol_type = other DNA
cccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 11 - DNA, 1214 bp, synthetic construct
  misc_feature 1..1214: note = Synthetic Nucleic Acid
  source 1..1214: mol_type = other DNA
gctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgattaactgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgaattaatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattgcggtaagtatcaaggttacaagacaggtttaaggagaccaatagaaactgggcttgtcgagacagagaagactcttgcgtttctgataggcacctattggtcttactgacatccactttgcctttctctccacagacctgggacagtgaat

SEQ ID NO: 12 - DNA, 1214 bp, synthetic construct; identical to SEQ ID NO: 11
  misc_feature 1..1214: note = Synthetic Nucleic Acid
  source 1..1214: mol_type = other DNA

SEQ ID NO: 13 - DNA, 861 bp, synthetic construct
  misc_feature 1..861: note = Synthetic Nucleic Acid
  source 1..861: mol_type = other DNA
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 14 - DNA, 146 bp, synthetic construct
  misc_feature 1..146: note = Synthetic Nucleic Acid
  source 1..146: mol_type = other DNA
gttaatcattaagtcgttaatttttgtggcccttgcgatgtttgctctggttaataatctcaggacaaacagaggttaataattttccagatctctctgagcaatagtataaaaggccagcagcagcctgaccacatctcatcctc

SEQ ID NO: 15 - DNA, 716 bp, synthetic construct
  misc_feature 1..716: note = Synthetic Nucleic Acid
  source 1..716: mol_type = other DNA
gctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgagatctgctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 16 - DNA, 1224 bp, synthetic construct
  misc_feature 1..1224: note = Synthetic Nucleic Acid
  source 1..1224: mol_type = other DNA
tcgtcgacgggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgattaactgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgaattaatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattgcggtaagtatcaaggttacaagacaggtttaaggagaccaatagaaactgggcttgtcgagacagagaagactcttgcgtttctgataggcacctattggtcttactgacatccactttgcctttctctccacagacctgggacagtgaat

SEQ ID NO: 17 - protein, 750 aa, synthetic construct
  REGION 1..750: note = Synthetic Nucleic Acid
MLRAKNQLFLLSPHYLRQVKESSGSRLIQQRLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 18 - protein, 749 aa, synthetic construct
  REGION 1..749: note = Synthetic Nucleic Acid
LRAKNQLFLLSPHYLRQVKESSGSRLIQQRLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 19 - DNA, 2250 bp, synthetic construct
  misc_feature 1..2250: note = Synthetic Nucleic Acid
  source 1..2250: mol_type = other DNA
atgctgcgggccaagaatcaactgttcctgctgtcccctcactacctgcggcaagtgaaagagagcagcggcagcagactgatccagcagagactgctgcatcagcagcagccactgcaccctgaatgggccgctctggctaagaagcagctcaagggcaagaaccccgaggacctgatctggcacacaccagagggcatcagcatcaagcccctgtactccaagcgggacacaatggatctgcccgaggaactgcctggcgtgaagccttttacaagaggcccctatcctaccatgtataccttcagaccctggaccatccggcagtacgccggcttttctaccgtggaagagagcaacaagttctacaaggacaacatcaaggccggccagcagggactgagcgtggcatttgatctggctacccacaggggctacgacagcgacaaccctagagtgcggggagatgttggaatggccggcgtggcaatcgacacagtggaagataccaagatcctgttcgacggcatccctctggaaaagatgagcgtgtccatgaccatgaacggcgctgtgatccccgtgctggctaactttattgtgaccggcgaggaacagggcgtgcccaaagaaaagctgaccggcaccatccagaacgacatcctgaaagagttcatggttcgaaacacctacatcttcccacctgagccgagcatgaagatcattgccgacatcttcgagtacaccgccaagcacatgcccaagttcaacagcatctccatcagcggctaccacatgcaagaggctggcgccgatgccatcctggaactggcttatacactggccgacggcctggaatactccagaacaggactgcaagccggcctgaccatcgatgagtttgcccctagactgagcttcttctggggcatcggcatgaacttctacatggaaatcgccaagatgagagccggcagacggctgtgggctcacctgatcgagaagatgttccagcctaagaacagcaagagcctgctcctgagagcccactgtcagacaagtggctggtccctgactgagcaggacccctacaacaacatcgtgcgcacagccatcgaagctatggccgccgtgtttggcggaacacagagcctgcacaccaacagctttgacgaggctctgggcctgcctaccgtgaagtctgccagaatcgcccggaacacccagatcatcatccaagaggaaagcggcatccccaaggtggcagatccttggggcggcagctacatgatggaatgcctgaccaacgacgtgtacgacgccgctctgaagctgatcaacgagatcgaagagatgggcggcatggctaaggctgtggccgagggaatccccaagctgagaatcgaggaatgcgccgccagacggcaggccagaattgatagcggaagcgaagtgatcgtgggcgtgaacaagtaccagctcgaaaaagaggacgccgtcgaggtcctggctatcgacaataccagcgtgcggaaccggcagattgagaagctgaagaagatcaagagcagccgcgatcaggccctggccgaaagatgtcttgctgccctgacagagtgtgccgccagcggcgacggaaatattctggctctggccgtggatgccagccgggctagatgtaccgtgggcgagattacagacgccctgaagaaggtgttcggcgagcacaaggccaacgacagaatggtgtctggcgcctacagacaagagtttggcgagagcaaagagatcaccagcgccatcaagcgggtccacaagttcatggaaagagaaggcaggcggcccagactgctggtggctaagatgggacaagacggccatgacagaggcgccaaagtgatcgccacaggctttgccgatctgggcttcgacgtggacatcggccctctgtttcagacccctagagaggtggcacagcaggccgttgatgccgatgttcacgctgtgggcatctctacactggctgccggacacaagacactggtgcccgaactgatcaaagagctgaacagcctgggcagacccgacatccttgtgatgtgtggcggagtgatcccaccgcaggactacgagttcctgtttgaagtgggcgtgtccaacgtgttcggccctggcacaagaatccctaaagccgccgtgcaggttctggacgacatcgagaagtgcctggaaaaaaagcagcagagcgtg

SEQ ID NO: 20 - DNA, 2247 bp, synthetic construct
  misc_feature 1..2247: note = Synthetic Nucleic Acid
  source 1..2247: mol_type = other DNA
ctgcgggccaagaatcaactgttcctgctgtcccctcactacctgcggcaagtgaaagagagcagcggcagcagactgatccagcagagactgctgcatcagcagcagccactgcaccctgaatgggccgctctggctaagaagcagctcaagggcaagaaccccgaggacctgatctggcacacaccagagggcatcagcatcaagcccctgtactccaagcgggacacaatggatctgcccgaggaactgcctggcgtgaagccttttacaagaggcccctatcctaccatgtataccttcagaccctggaccatccggcagtacgccggcttttctaccgtggaagagagcaacaagttctacaaggacaacatcaaggccggccagcagggactgagcgtggcatttgatctggctacccacaggggctacgacagcgacaaccctagagtgcggggagatgttggaatggccggcgtggcaatcgacacagtggaagataccaagatcctgttcgacggcatccctctggaaaagatgagcgtgtccatgaccatgaacggcgctgtgatccccgtgctggctaactttattgtgaccggcgaggaacagggcgtgcccaaagaaaagctgaccggcaccatccagaacgacatcctgaaagagttcatggttcgaaacacctacatcttcccacctgagccgagcatgaagatcattgccgacatcttcgagtacaccgccaagcacatgcccaagttcaacagcatctccatcagcggctaccacatgcaagaggctggcgccgatgccatcctggaactggcttatacactggccgacggcctggaatactccagaacaggactgcaagccggcctgaccatcgatgagtttgcccctagactgagcttcttctggggcatcggcatgaacttctacatggaaatcgccaagatgagagccggcagacggctgtgggctcacctgatcgagaagatgttccagcctaagaacagcaagagcctgctcctgagagcccactgtcagacaagtggctggtccctgactgagcaggacccctacaacaacatcgtgcgcacagccatcgaagctatggccgccgtgtttggcggaacacagagcctgcacaccaacagctttgacgaggctctgggcctgcctaccgtgaagtctgccagaatcgcccggaacacccagatcatcatccaagaggaaagcggcatccccaaggtggcagatccttggggcggcagctacatgatggaatgcctgaccaacgacgtgtacgacgccgctctgaagctgatcaacgagatcgaagagatgggcggcatggctaaggctgtggccgagggaatccccaagctgagaatcgaggaatgcgccgccagacggcaggccagaattgatagcggaagcgaagtgatcgtgggcgtgaacaagtaccagctcgaaaaagaggacgccgtcgaggtcctggctatcgacaataccagcgtgcggaaccggcagattgagaagctgaagaagatcaagagcagccgcgatcaggccctggccgaaagatgtcttgctgccctgacagagtgtgccgccagcggcgacggaaatattctggctctggccgtggatgccagccgggctagatgtaccgtgggcgagattacagacgccctgaagaaggtgttcggcgagcacaaggccaacgacagaatggtgtctggcgcctacagacaagagtttggcgagagcaaagagatcaccagcgccatcaagcgggtccacaagttcatggaaagagaaggcaggcggcccagactgctggtggctaagatgggacaagacggccatgacagaggcgccaaagtgatcgccacaggctttgccgatctgggcttcgacgtggacatcggccctctgtttcagacccctagagaggtggcacagcaggccgttgatgccgatgttcacgctgtgggcatctctacactggctgccggacacaagacactggtgcccgaactgatcaaagagctgaacagcctgggcagacccgacatccttgtgatgtgtggcggagtgatcccaccgcaggactacgagttcctgtttgaagtgggcgtgtccaacgtgttcggccctggcacaagaatccctaaagccgccgtgcaggttctggacgacatcgagaagtgcctggaaaaaaagcagcagagcgtg

SEQ ID NO: 21 - protein, 354 aa, synthetic construct
  REGION 1..354: note = Synthetic Nucleic Acid
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 22 - DNA, 1062 bp, synthetic construct
  misc_feature 1..1062: note = Synthetic Nucleic Acid
  source 1..1062: mol_type = other DNA
atgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttc

SEQ ID NO: 23 - DNA, 1062 bp, synthetic construct
  misc_feature 1..1062: note = Synthetic Nucleic Acid
  source 1..1062: mol_type = other DNA
atgctgttcaacctgcgaatcctgctgaacaatgccgcttttcggaacgggcacaatttcatggtgaggaactttcgctgcggacagcccctccagaacaaggtccagctgaagggcagggacctgctgaccctgaaaaatttcacaggggaggaaatcaagtacatgctgtggctgtcagccgatctgaagttccggatcaagcagaagggcgaatatctgcctctgctccagggcaaaagcctggggatgatcttcgaaaagcgcagtactcggaccagactgtcaacagagactggattcgcactgctgggaggacacccatgttttctgaccacacaggacattcatctgggagtgaacgagtccctgaccgacacagcacgcgtcctgagctccatggctgatgcagtgctggctcgagtctacaaacagtctgacctggataccctggccaaggaagcttctatcccaatcattaatggcctgagtgacctgtatcaccccatccagattctggccgattacctgaccctccaggagcattattctagtctgaaagggctgacactgagctggattggggacggaaacaatatcctgcactccattatgatgagcgccgccaagtttggaatgcacctccaggctgcaaccccaaaaggctacgaacccgatgcctccgtgacaaagctggcagaacagtatgccaaagagaacggcactaagctgctgctcaccaatgaccctctggaggccgctcacggaggcaacgtgctgatcactgatacctggattagtatgggacaggaggaagagaagaagaagcggctccaggccttccagggctaccaggtgacaatgaaaactgctaaggtcgcagccagcgactggacctttctgcattgcctgcccagaaagcctgaagaggtggacgatgaggtcttctactcacccagaagcctggtgtttcctgaagctgagaataggaagtggacaatcatggcagtgatggtcagcctgctgactgattattcccctcagctccagaaaccaaagttc

SEQ ID NO: 24 - protein, 395 aa, synthetic construct
  REGION 1..395: note = Synthetic Nucleic Acid
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 25 - DNA, 1185 bp, synthetic construct
  misc_feature 1..1185: note = Synthetic Nucleic Acid
  source 1..1185: mol_type = other DNA
atgggggtccaggtggaaacaatctctccgggggatgggcggacattccctaaaaggggccagacctgcgtggtgcattacaccggcatgctggaagatggcaagaaggtggacagcagccgggacagaaacaagcccttcaagttcatgctgggcaagcaagaagtgatcagaggctgggaagagggcgtcgcccagatgtctgttggacagagagccaagctgacaatcagccccgattacgcctatggcgccacaggacaccctggcatcattcctccacatgccacactggtgttcgacgtggaactgctgaagctggaaggcggcggaggatctggctttggagatgtgggagccctggaaagcctgagaggcaatgccgatctggcctacatcctgagcatggaaccttgcggccactgcctgattatcaacaacgtgaacttctgtagagagagcggcctgcggaccagaaccggcagcaatatcgattgcgagaagctgcggcggagattcagcagcctgcacttcatggtggaagtgaagggcgacctgaccgccaagaaaatggtgctggctctgctggaactggcccagcaagatcatggcgccctggattgctgtgtggtcgtgatcctgtctcacggctgtcaggccagccaccttcaattccctggcgccgtgtatggcacagatggctgtcctgtgtccgtggaaaagatcgtgaacatcttcaacggcaccagctgtcctagcctcggcggaaagcccaagctgttcttcatccaagcctgtggcggcgagcagaaggatcacggatttgaggtggccagcacaagccccgaggatgagtctcctggaagcaaccctgagcctgacgccacacctttccaagagggcctgagaaccttcgaccagctggacgctatcagctccctgcctacacctagcgacatcttcgtgtcctacagcacattccccggctttgtgtcttggcgggaccctaagtctggctcttggtacgtggaaaccctggacgacatctttgagcagtgggctcacagcgaggacctccagtctctgctgctgagagtggccaatgccgtgtccgtgaagggcatctacaagcagatgcctggctgcttcaacttcctgcggaagaagctgtttttcaagaccagc

SEQ ID NO: 26 - DNA, 139 bp, synthetic construct
  misc_feature 1..139: note = Synthetic Nucleic Acid
  source 1..139: mol_type = other DNA
attccgataacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 27 - DNA, 192 bp, synthetic construct
  misc_feature 1..192: note = Synthetic Nucleic Acid
  source 1..192: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttt

SEQ ID NO: 28 - protein, 18 aa, synthetic construct
  REGION 1..18: note = Synthetic Nucleic Acid
EGRGSLLTCGDVEENPGP

SEQ ID NO: 29 - protein, 21 aa, synthetic construct
  REGION 1..21: note = Synthetic Nucleic Acid
GSGEGRGSLLTCGDVEENPGP

SEQ ID NO: 30 - DNA, 63 bp, synthetic construct
  misc_feature 1..63: note = Synthetic Nucleic Acid
  source 1..63: mol_type = other DNA
ggatctggagagggaaggggaagcctgctgacctgtggagacgtggaggaaaacccaggacca

SEQ ID NO: 31 - DNA, 63 bp, synthetic construct
  misc_feature 1..63: note = Synthetic Nucleic Acid
  source 1..63: mol_type = other DNA
ggatccggcgaaggcagaggctcactgcttacttgtggcgacgtggaggagaaccccggacct

SEQ ID NO: 32 - DNA, 63 bp, synthetic construct
  misc_feature 1..63: note = Synthetic Nucleic Acid
  source 1..63: mol_type = other DNA
ggctccggagaaggacggggaagcctgcttacatgcggagatgtggaggagaatcctggtccc

SEQ ID NO: 33 - protein, 20 aa, synthetic construct
  REGION 1..20: note = Synthetic Nucleic Acid
QCTNYALLKLAGDVESNPGP

SEQ ID NO: 34 - protein, 23 aa, synthetic construct
  REGION 1..23: note = Synthetic Nucleic Acid
GSGQCTNYALLKLAGDVESNPGP

SEQ ID NO: 35 - protein, 22 aa, synthetic construct
  REGION 1..22: note = Synthetic Nucleic Acid
VKQTLNFDLLKLAGDVESNPGP

SEQ ID NO: 36 - protein, 25 aa, synthetic construct
  REGION 1..25: note = Synthetic Nucleic Acid
GSGVKQTLNFDLLKLAGDVESNPGP

SEQ ID NO: 37 - protein, 19 aa, synthetic construct
  REGION 1..19: note = Synthetic Nucleic Acid
ATNFSLLKQAGDVEENPGP

SEQ ID NO: 38 - protein, 22 aa, synthetic construct
  REGION 1..22: note = Synthetic Nucleic Acid
GSGATNFSLLKQAGDVEENPGP

SEQ ID NO: 39 - protein, 594 aa, synthetic construct
  REGION 1..594: note = Synthetic Nucleic Acid
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 40 - protein, 594 aa, synthetic construct
  REGION 1..594: note = Synthetic Nucleic Acid
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 41 - protein, 593 aa, synthetic construct
  REGION 1..593: note = Synthetic Nucleic Acid
GSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 42 - protein, 593 aa, synthetic construct
  REGION 1..593: note = Synthetic Nucleic Acid
GSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 43 - protein, 7 aa, synthetic construct
  REGION 1..7: note = Synthetic Nucleic Acid
PKKKRKV

SEQ ID NO: 44 - DNA, 21 bp, synthetic construct
  misc_feature 1..21: note = Synthetic Nucleic Acid
  source 1..21: mol_type = other DNA
cccaagaagaagcggaaagtt

SEQ ID NO: 45 - protein, 5 aa, synthetic construct
  REGION 1..5: note = Synthetic Nucleic Acid
GGGGS

SEQ ID NO: 46 - DNA, 15 bp, synthetic construct
  misc_feature 1..15: note = Synthetic Nucleic Acid
  source 1..15: mol_type = other DNA
ggcggcggaggcagc

SEQ ID NO: 47 - protein, 605 aa, synthetic construct
  REGION 1..605: note = Synthetic Nucleic Acid
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 48 - DNA, 1815 bp, synthetic construct
  misc_feature 1..1815: note = Synthetic Nucleic Acid
  source 1..1815: mol_type = other DNA
atggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttc

SEQ ID NO: 49 - protein, 603 aa, synthetic construct
  REGION 1..603: note = Synthetic Nucleic Acid
PKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 50 - DNA, 1809 bp, synthetic construct
  misc_feature 1..1809: note = Synthetic Nucleic Acid
  source 1..1809: mol_type = other DNA
cccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttc

SEQ ID NO: 51 - protein, 340 aa, synthetic construct
  REGION 1..340: note = Synthetic Nucleic Acid
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 52 - protein, 339 aa, synthetic construct
  REGION 1..339: note = Synthetic Nucleic Acid
GKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 53 - protein, 340 aa, synthetic construct
  REGION 1..340: note = Synthetic Nucleic Acid
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 54 - protein, 339 aa, synthetic construct
  REGION 1..339: note = Synthetic Nucleic Acid
GKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 55 - protein, 1496 aa, synthetic construct
  REGION 1..1496: note = Synthetic Nucleic Acid
MSKEQLLIQRSSAAERCRRYRQKMSAEQRASDLERRRRLQQNVSEEQLLEKRRSEAEKQRRHRQKMSKDQRAFEVERRRWRRQNMSREQSSTSTTNTGRNCLLSKNGVHEDAILEHSCGGMTVRCEFCLSLNFSDEKPSDGKFTRCCSKGKVCPNDIHFPDYPAYLKRLMTNEDSDSKNFMENIRSINSSFAFASMGANIASPSGYGPYCFRIHGQVYHRTGTLHPSDGVSRKFAQLYILDTAEATSKRLAMPENQGCSERLMININNLMHEINELTKSYKMLHEVEKEAQSEAAAKGIAPTEVTMAIKYDRNSDPGRYNSPRVTEVAVIFRNEDGEPPFERDLLIHCKPDPNNPNATKMKQISILFPTLDAMTYPILFPHGEKGWGTDIALRLRDNSVIDNNTRQNVRTRVTQMQYYGFHLSVRDTFNPILNAGKLTQQFIVDSYSKMEANRINFIKANQSKLRVEKYSGLMDYLKSRSENDNVPIGKMIILPSSFEGSPRNMQQRYQDAMAIVTKYGKPDLFITMTCNPKWADITNNLQRWQKVENRPDLVARVFNIKLNALLNDICKFHLFGKVIAKIHVIEFQKRGLPHAHILLILDSESKLRSEDDIDRIVKAEIPDEDQCPRLFQIVKSNMVHGPCGIQNPNSPCMENGKCSKGYPKEFQNATIGNIDGYPKYKRRSGSTMSIGNKVVDNTWIVPYNPYLCLKYNCHINVEVCASIKSVKYLFKYIYKGHDCANIQISEKNIINHDEVQDFIDSRYVSAPEAVWRLFAMRMHDQSHAITRLAIHLPNDQNLYFHTDDFAEVLDRAKRHNSTLMAWFLLNREDSDARNYYYWEIPQHYVFNNSLWTKRRKGGNKVLGRLFTVSFREPERYYLRLLLLHVKGAISFEDLRTVGGVTYDTFHEAAKHRGLLLDDTIWKDTIDDAIILNMPKQLRQLFAYICVFGCPSAADKLWDENKSHFIEDFCWKLHRREGACVNCEMHALNEIQEVFTLHGMKCSHFKLPDYPLLMNANTCDQLYEQQQAEVLINSLNDEQLAAFQTITSAIEDQTVHPKCFFLDGPGGSGKTYLYKVLTHYIRGRGGTVLPTASTGIAANLLLGGRTFHSQYKLPIPLNETSISRLDIKSEVAKTIKKAQLLIIDECTMASSHAINAIDRLLREIMNLNVAFGGKVLLLGGDFRQCLSIVPHAMRSAIVQTSLKYCNVWGCFRKLSLKTNMRSEDSAYSEWLVKLGDGKLDSSFHLGMDIIEIPHEMICNGSIIEATFGNSISIDNIKNISKRAILCPKNEHVQKLNEEILDILDGDFHTYLSDDSIDSTDDAEKENFPIEFLNSITPSGMPCHKLKLKVGAIIMLLRNLNSKWGLCNGTRFIIKRLRPNIIEAEVLTGSAEGEVVLIPRIDLSPSDTGLPFKLIRRQFPVMPAFAMTINKSQGQTLDRVGIFLPEPVFAHGQLYVAFSRVRRACDVKVKVVNTSSQGKLVKHSESVFTLNVVYREILE

SEQ ID NO: 56 - protein, 1495 aa, synthetic construct
  REGION 1..1495: note = Synthetic Nucleic Acid
SKEQLLIQRSSAAERCRRYRQKMSAEQRASDLERRRRLQQNVSEEQLLEKRRSEAEKQRRHRQKMSKDQRAFEVERRRWRRQNMSREQSSTSTTNTGRNCLLSKNGVHEDAILEHSCGGMTVRCEFCLSLNFSDEKPSDGKFTRCCSKGKVCPNDIHFPDYPAYLKRLMTNEDSDSKNFMENIRSINSSFAFASMGANIASPSGYGPYCFRIHGQVYHRTGTLHPSDGVSRKFAQLYILDTAEATSKRLAMPENQGCSERLMININNLMHEINELTKSYKMLHEVEKEAQSEAAAKGIAPTEVTMAIKYDRNSDPGRYNSPRVTEVAVIFRNEDGEPPFERDLLIHCKPDPNNPNATKMKQISILFPTLDAMTYPILFPHGEKGWGTDIALRLRDNSVIDNNTRQNVRTRVTQMQYYGFHLSVRDTFNPILNAGKLTQQFIVDSYSKMEANRINFIKANQSKLRVEKYSGLMDYLKSRSENDNVPIGKMIILPSSFEGSPRNMQQRYQDAMAIVTKYGKPDLFITMTCNPKWADITNNLQRWQKVENRPDLVARVFNIKLNALLNDICKFHLFGKVIAKIHVIEFQKRGLPHAHILLILDSESKLRSEDDIDRIVKAEIPDEDQCPRLFQIVKSNMVHGPCGIQNPNSPCMENGKCSKGYPKEFQNATIGNIDGYPKYKRRSGSTMSIGNKVVDNTWIVPYNPYLCLKYNCHINVEVCASIKSVKYLFKYIYKGHDCANIQISEKNIINHDEVQDFIDSRYVSAPEAVWRLFAMRMHDQSHAITRLAIHLPNDQNLYFHTDDFAEVLDRAKRHNSTLMAWFLLNREDSDARNYYYWEIPQHYVFNNSLWTKRRKGGNKVLGRLFTVSFREPERYYLRLLLLHVKGAISFEDLRTVGGVTYDTFHEAAKHRGLLLDDTIWKDTIDDAIILNMPKQLRQLFAYICVFGCPSAADKLWDENKSHFIEDFCWKLHRREGACVNCEMHALNEIQEVFTLHGMKCSHFKLPDYPLLMNANTCDQLYEQQQAEVLINSLNDEQLAAFQTITSAIEDQTVHPKCFFLDGPGGSGKTYLYKVLTHYIRGRGGTVLPTASTGIAANLLLGGRTFHSQYKLPIPLNETSISRLDIKSEVAKTIKKAQLLIIDECTMASSHAINAIDRLLREIMNLNVAFGGKVLLLGGDFRQCLSIVPHAMRSAIVQTSLKYCNVWGCFRKLSLKTNMRSEDSAYSEWLVKLGDGKLDSSFHLGMDIIEIPHEMICNGSIIEATFGNSISIDNIKNISKRAILCPKNEHVQKLNEEILDILDGDFHTYLSDDSIDSTDDAEKENFPIEFLNSITPSGMPCHKLKLKVGAIIMLLRNLNSKWGLCNGTRFIIKRLRPNIIEAEVLTGSAEGEVVLIPRIDLSPSDTGLPFKLIRRQFPVMPAFAMTINKSQGQTLDRVGIFLPEPVFAHGQLYVAFSRVRRACDVKVKVVNTSSQGKLVKHSESVFTLNVVYREILE

SEQ ID NO: 57 - protein, 649 aa, synthetic construct
  REGION 1..649: note = Synthetic Nucleic Acid
MEEVCDSSAAASSTVQNQPQDQEHPWPYLREFFSLSGVNKDSFKMKCVLCLPLNKEISAFKSSPSNLRKHIERMHPNYLKNYSKLTAQKRKIGTSTHASSSKQLKVDSVFPVKHVSPVTVNKAILRYIIQGLHPFSTVDLPSFKELISTLQPGISVITRPTLRSKIAEAALIMKQKVTAAMSEVEWIATTTDCWTARRKSFIGVTAHWINPGSLERHSAALACKRLMGSHTFEVLASAMNDIHSEYEIRDKVVCTTTDSGSNFMKAFRVFGVENNDIETEARRCESDDTDSEGCGEGSDGVEFQDASRVLDQDDGFEFQLPKHQKCACHLLNLVSSVDAQKALSNEHYKKLYRSVFGKCQALWNKSSRSALAAEAVESESRLQLLRPNQTRWNSTFMAVDRILQICKEAGEGALRNICTSLEVPMFNPAEMLFLTEWANTMRPVAKVLDILQAETNTQLGWLLPSVHQLSLKLQRLHHSLRYCDPLVDALQQGIQTRFKHMFEDPEIIAAAILLPKFRTSWTNDETIIKRGMDYIRVHLEPLDHKKELANSSSDDEDFFASLKPTTHEASKELDGYLACVSDTRESLLTFPAICSLSIKTNTPLPASAACERLFSTAGLLFSPKRARLDTNNFENQLLLKLNLRFYNFE

SEQ ID NO: 58 - protein, 648 aa, synthetic construct
  REGION 1..648: note = Synthetic Nucleic Acid
EEVCDSSAAASSTVQNQPQDQEHPWPYLREFFSLSGVNKDSFKMKCVLCLPLNKEISAFKSSPSNLRKHIERMHPNYLKNYSKLTAQKRKIGTSTHASSSKQLKVDSVFPVKHVSPVTVNKAILRYIIQGLHPFSTVDLPSFKELISTLQPGISVITRPTLRSKIAEAALIMKQKVTAAMSEVEWIATTTDCWTARRKSFIGVTAHWINPGSLERHSAALACKRLMGSHTFEVLASAMNDIHSEYEIRDKVVCTTTDSGSNFMKAFRVFGVENNDIETEARRCESDDTDSEGCGEGSDGVEFQDASRVLDQDDGFEFQLPKHQKCACHLLNLVSSVDAQKALSNEHYKKLYRSVFGKCQALWNKSSRSALAAEAVESESRLQLLRPNQTRWNSTFMAVDRILQICKEAGEGALRNICTSLEVPMFNPAEMLFLTEWANTMRPVAKVLDILQAETNTQLGWLLPSVHQLSLKLQRLHHSLRYCDPLVDALQQGIQTRFKHMFEDPEIIAAAILLPKFRTSWTNDETIIKRGMDYIRVHLEPLDHKKELANSSSDDEDFFASLKPTTHEASKELDGYLACVSDTRESLLTFPAICSLSIKTNTPLPASAACERLFSTAGLLFSPKRARLDTNNFENQLLLKLNLRFYNFE

SEQ ID NO: 59 - protein, 636 aa, synthetic construct
  REGION 1..636: note = Synthetic Nucleic Acid
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLENAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 60 - protein, 635 aa, synthetic construct
  REGION 1..635: note = Synthetic Nucleic Acid
MLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLENAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 61 - protein, 239 aa, synthetic construct
  REGION 1..239: note = Synthetic Nucleic Acid
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 62 - protein, 238 aa, synthetic construct
  REGION 1..238: note = Synthetic Nucleic Acid
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 63 - DNA, 717 bp, synthetic construct
  misc_feature 1..717: note = Synthetic Nucleic Acid
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgtgatcacatggtcctgctggagttcgtgaccgccgccgggatcactctcggcatggacgagctgtacaag

SEQ ID NO: 64 - DNA, 714 bp, synthetic construct
  misc_feature 1..714: note = Synthetic Nucleic Acid
  source 1..714: mol_type = other DNA
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgtgatcacatggtcctgctggagttcgtgaccgccgccgggatcactctcggcatggacgagctgtacaag

SEQ ID NO: 65 - protein, 550 aa, synthetic construct
  REGION 1..550: note = Synthetic Nucleic Acid
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 66 - protein, 549 aa, synthetic construct
  REGION 1..549: note = Synthetic Nucleic Acid
EDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 67 - DNA, 1650 bp, synthetic construct
  misc_feature 1..1650: note = Synthetic Nucleic Acid
  source 1..1650: mol_type = other DNA
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtg

SEQ ID NO: 68 - DNA, 1647 bp, synthetic construct
  misc_feature 1..1647: note = Synthetic Nucleic Acid
  source 1..1647: mol_type = other DNA
gaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtg

SEQ ID NO: 69 - DNA, 869 bp, synthetic construct
  misc_feature 1..869: note = Synthetic Nucleic Acid
  source 1..869: mol_type = other DNA
tgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagc

SEQ ID NO: 70 - DNA, 1866 bp, synthetic construct
  misc_feature 1..1866: note = Synthetic Nucleic Acid
  source 1..1866: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 71 - DNA, 2214 bp, synthetic construct
  misc_feature 1..2214: note = Synthetic Nucleic Acid
  source 1..2214: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttccgggttacaaatacctcggacccggcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaacctgttaagacggctccgggaaaaaagaggccggtagagcactctcctgtggagccagactcctcctcgggaaccggaaaggcgggccagcagcctgcgaggaagcgactcaactttggtcagactggagacaccgactccgccgctgacccccagcctctcggagaaccaccagcagccccctctggtctgggaactggtacaatggctgcaggcggtggcgctccaatggcagacaataacgaaggcgccgacggagtgggtaatgcctcgggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtactttgacttcaaccgcttccactgccacttctcaccacgtgactggcaaagactcatcaacaacaactggggattccggcccaagagactcaacttcaagctcttcaacatccaggtcaaggaggtcacgcagaatgaaggcaccaagaccatcgccaataaccttaccagcacggttcaggtgtttactgactcggagtaccagctgccgtacgttctcggctctgcccaccagggctgcctgcctccgttcccggcggacgtgttcatgattccgcagtacggctacctaacgctcaacaatggcagccaggcgatgggtcgctcgtccttctactgcctggagtactttccgtcgcagatgctgagaaccggcaacaacttccagtttacttacaccttcgaggacgtgcctttccacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggcggctttggcctgaaacatcctccgcctcagatcctgatcaagaacacgcctgtacctgcggatcctccaacggccttcaacaaggacaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 72 - DNA, 1866 bp, synthetic construct
  misc_feature 1..1866: note = Synthetic Nucleic Acid
  source 1..1866: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 73 - DNA, 2208 bp, synthetic construct
  misc_feature 1..2208: note = Synthetic Nucleic Acid
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggcaagacaggccagcagcccgctaaaaagagactcaattttggtcagactggcgactcagagtcagtcccagaccctcaacctctcggagaaccaccagcagccccctctggtctgggaactaatacgatggctacaggcagtggcgcaccaatggcagacaataacgagggtgccgatggagtgggtaattcctcaggaaattggcattgcgattcccaatggctgggcgacagagtcatcaccaccagcacccgaacctgggccctgcccacctacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtgggctggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcgacacacaaggcgttcttccaggcatggtatggcaggacagagatgtgtaccttcagggacccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactcaacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 74 - DNA, 16 bp, synthetic construct
  misc_feature 1..16: note = Synthetic Nucleic Acid
  source 1..16: mol_type = other DNA
atgatttaaatcaggt

SEQ ID NO: 75 - DNA, 5336 bp, synthetic construct
  misc_feature 1..5336: note = Synthetic Nucleic Acid
  source 1..5336: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcg

SEQ ID NO: 76 - DNA, 3944 bp, synthetic construct
  misc_feature 1..3944: note = Synthetic Nucleic Acid
  source 1..3944: mol_type = other DNA
cccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacg

SEQ ID NO: 77 - DNA, 77 bp, synthetic construct
  misc_feature 1..77: note = Synthetic Nucleic Acid
  source 1..77: mol_type = other DNA
cccagggatgtacgtccctaacccgctagggggcagcacccaggcctgcactgccgcctgccggcaggggtccagtc

SEQ ID NO: 78 - DNA, 77 bp, synthetic construct
  misc_feature 1..77: note = Synthetic Nucleic Acid
  source 1..77: mol_type = other DNA
gactggacccctgccggcaggcggcagtgcaggcctgggtgctgccccctagcgggttagggacgtacatccctggg

SEQ ID NO: 79 - DNA, 190 bp, synthetic construct
  misc_feature 1..190: note = Synthetic Nucleic Acid
  source 1..190: mol_type = other DNA
gtgcgggccaggcccccgagggccttatcggccccagaggcgcttgctgtcgggccgggcgctcccggcacgggcgggcggaggggtggcgcccgcctggggaccgcagattacaagagcacctcctcccccaaccccaggaggccccgctccccaggcctcggccggcgcggacccctggttgccccgg

SEQ ID NO: 80 - DNA, 190 bp, synthetic construct
  misc_feature 1..190: note = Synthetic Nucleic Acid
  source 1..190: mol_type = other DNA
ccggggcaaccaggggtccgcgccggccgaggcctggggagcggggcctcctggggttgggggaggaggtgctcttgtaatctgcggtccccaggcgggcgccacccctccgcccgcccgtgccgggagcgcccggcccgacagcaagcgcctctggggccgataaggccctcgggggcctggcccgcac

SEQ ID NO: 81 - protein, 353 aa, synthetic construct
  REGION 1..353: note = Synthetic Nucleic Acid
LFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 82 - DNA, 1059 bp, synthetic construct
  misc_feature 1..1059: note = Synthetic Nucleic Acid
  source 1..1059: mol_type = other DNA
ctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttc

SEQ ID NO: 83 - DNA, 1059 bp, synthetic construct
  misc_feature 1..1059: note = Synthetic Nucleic Acid
  source 1..1059: mol_type = other DNA
ctgttcaacctgcgaatcctgctgaacaatgccgcttttcggaacgggcacaatttcatggtgaggaactttcgctgcggacagcccctccagaacaaggtccagctgaagggcagggacctgctgaccctgaaaaatttcacaggggaggaaatcaagtacatgctgtggctgtcagccgatctgaagttccggatcaagcagaagggcgaatatctgcctctgctccagggcaaaagcctggggatgatcttcgaaaagcgcagtactcggaccagactgtcaacagagactggattcgcactgctgggaggacacccatgttttctgaccacacaggacattcatctgggagtgaacgagtccctgaccgacacagcacgcgtcctgagctccatggctgatgcagtgctggctcgagtctacaaacagtctgacctggataccctggccaaggaagcttctatcccaatcattaatggcctgagtgacctgtatcaccccatccagattctggccgattacctgaccctccaggagcattattctagtctgaaagggctgacactgagctggattggggacggaaacaatatcctgcactccattatgatgagcgccgccaagtttggaatgcacctccaggctgcaaccccaaaaggctacgaacccgatgcctccgtgacaaagctggcagaacagtatgccaaagagaacggcactaagctgctgctcaccaatgaccctctggaggccgctcacggaggcaacgtgctgatcactgatacctggattagtatgggacaggaggaagagaagaagaagcggctccaggccttccagggctaccaggtgacaatgaaaactgctaaggtcgcagccagcgactggacctttctgcattgcctgcccagaaagcctgaagaggtggacgatgaggtcttctactcacccagaagcctggtgtttcctgaagctgagaataggaagtggacaatcatggcagtgatggtcagcctgctgactgattattcccctcagctccagaaaccaaagttc

SEQ ID NO: 84 - protein, 394 aa, synthetic construct
  REGION 1..394: note = Synthetic Nucleic Acid
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 85 - DNA, 1182 bp, synthetic construct
  misc_feature 1..1182: note = Synthetic Nucleic Acid
  source 1..1182: mol_type = other DNA
ggggtccaggtggaaacaatctctccgggggatgggcggacattccctaaaaggggccagacctgcgtggtgcattacaccggcatgctggaagatggcaagaaggtggacagcagccgggacagaaacaagcccttcaagttcatgctgggcaagcaagaagtgatcagaggctgggaagagggcgtcgcccagatgtctgttggacagagagccaagctgacaatcagccccgattacgcctatggcgccacaggacaccctggcatcattcctccacatgccacactggtgttcgacgtggaactgctgaagctggaaggcggcggaggatctggctttggagatgtgggagccctggaaagcctgagaggcaatgccgatctggcctacatcctgagcatggaaccttgcggccactgcctgattatcaacaacgtgaacttctgtagagagagcggcctgcggaccagaaccggcagcaatatcgattgcgagaagctgcggcggagattcagcagcctgcacttcatggtggaagtgaagggcgacctgaccgccaagaaaatggtgctggctctgctggaactggcccagcaagatcatggcgccctggattgctgtgtggtcgtgatcctgtctcacggctgtcaggccagccaccttcaattccctggcgccgtgtatggcacagatggctgtcctgtgtccgtggaaaagatcgtgaacatcttcaacggcaccagctgtcctagcctcggcggaaagcccaagctgttcttcatccaagcctgtggcggcgagcagaaggatcacggatttgaggtggccagcacaagccccgaggatgagtctcctggaagcaaccctgagcctgacgccacacctttccaagagggcctgagaaccttcgaccagctggacgctatcagctccctgcctacacctagcgacatcttcgtgtcctacagcacattccccggctttgtgtcttggcgggaccctaagtctggctcttggtacgtggaaaccctggacgacatctttgagcagtgggctcacagcgaggacctccagtctctgctgctgagagtggccaatgccgtgtccgtgaagggcatctacaagcagatgcctggctgcttcaacttcctgcggaagaagctgtttttcaagaccagc

SEQ ID NO: 86 - DNA, 309 bp, synthetic construct
  misc_feature 1..309: note = Synthetic Nucleic Acid
  source 1..309: mol_type = other DNA
gatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctaggg

SEQ ID NO: 87 - DNA, 35 bp, synthetic construct
  misc_feature 1..35: note = Synthetic Nucleic Acid
  source 1..35: mol_type = other DNA
ccctagaaagatagtctgcgtaaaattgacgcatg

SEQ ID NO: 88 - DNA, 63 bp, synthetic construct
  misc_feature 1..63: note = Synthetic Nucleic Acid
  source 1..63: mol_type = other DNA
ccctagaaagataatcatattgtgacgtacgttaaagataatcatgcgtaaaattgacgcatg

SEQ ID NO: 89 - DNA, 64 bp, synthetic construct
  misc_feature 1..64: note = Synthetic Nucleic Acid
  source 1..64: mol_type = other DNA
ccctagaaagataatcatattgtgacgtacgttaaagataatcatgcgtaaaattgacgcatgc

SEQ ID NO: 90 - DNA, 237 bp, synthetic construct
  misc_feature 1..237: note = Synthetic Nucleic Acid
  source 1..237: mol_type = other DNA
ccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaactttta

SEQ ID NO: 91 - DNA, 94 bp, synthetic construct
  misc_feature 1..94: note = Synthetic Nucleic Acid
  source 1..94: mol_type = other DNA
ggtaccgtgcacgtcgactctagacgcgtagaggggcggaagggacgttaggagggaggcagggaggcagggaggcagggaggaacggagggag

SEQ ID NO: 92 - DNA, 94 bp, synthetic construct
  misc_feature 1..94: note = Synthetic Nucleic Acid
  source 1..94: mol_type = other DNA
ctccctccgttcctccctgcctccctgcctccctgcctccctcctaacgtcccttccgcccctctacgcgtctagagtcgacgtgcacggtacc

SEQ ID NO: 93 - DNA, 119 bp, synthetic construct
  misc_feature 1..119: note = Synthetic Nucleic Acid
  source 1..119: mol_type = other DNA
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 94 - DNA, 133 bp, synthetic construct
  misc_feature 1..133: note = Synthetic Nucleic Acid
  source 1..133: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctg

SEQ ID NO: 95 - DNA, 238 bp, synthetic construct; identical to SEQ ID NO: 6
  misc_feature 1..238: note = Synthetic Nucleic Acid
  source 1..238: mol_type = other DNA

SEQ ID NO: 96 - DNA, 309 bp, synthetic construct; identical to SEQ ID NO: 86
  misc_feature 1..309: note = Synthetic Nucleic Acid
  source 1..309: mol_type = other DNA

SEQ ID NO: 97 - DNA, 122 bp, synthetic construct
  misc_feature 1..122: note = Synthetic Nucleic Acid
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 98 - DNA, 20 bp, synthetic construct
  misc_feature 1..20: note = Synthetic Nucleic Acid
  source 1..20: mol_type = other DNA
aagctgcaataaacaagtta

SEQ ID NO: 99 - DNA, 15 bp, synthetic construct
  misc_feature 1..15: note = Synthetic Nucleic Acid
  source 1..15: mol_type = other DNA
ctgcgcatgcgcttg

SEQ ID NO: 100 - DNA, 21 bp, synthetic construct
  misc_feature 1..21: note = Synthetic Nucleic Acid
  source 1..21: mol_type = other DNA
gtaatcatggtcatagctgtt

SEQ ID NO: 101 - DNA, 18 bp, synthetic construct
  misc_feature 1..18: note = Synthetic Nucleic Acid
  source 1..18: mol_type = other DNA
agctagccttaagggcgc

SEQ ID NO: 102 - DNA, 34 bp, synthetic construct
  misc_feature 1..34: note = Synthetic Nucleic Acid
  source 1..34: mol_type = other DNA
acagtgtacatcatgaccatggtctagagtagac

SEQ ID NO: 103 - DNA, 977 bp, synthetic construct
  misc_feature 1..977: note = Synthetic Nucleic Acid
  source 1..977: mol_type = other DNA
acagtgtacatcatgaccatggtctagagtagacgataaccacactgacgctaagacatcgttatttttttagacactttaccctctgttattatccccgccgggaatggctacccggatcatatacaccgacgtgccaagttcccgccctgtctccttatttccgctcggtgcaagattacagacgtccgaagtgtattagagtagacgcatcagtatcacttgtgtccttaaaccgacaggagaagtgctagtacgctgtgtaaaggatcctgaatataatataggacaccgtctattatatgtatataggaaacttatgtcgtatttgggcggaccaccaatccgattccaaaccccggttggaacgtgtcggatcgacactcgcccaaaatgaaatttaggctcttaaatgcaaaatctggttcgtaaattcagtcgggatagagacagttaacggttgcttgtctccatgacctacacatggtgcagggtgtcttggggataccagattgttatataacggtgatcaaacaagtagttccaaaagaaatatacagaagatccacagctaggccatatcctttaagagttagttatcacagcaacctcgctaactggcgtagagcgtaagccgcggttcgcaggtcataccactttatgattagtccttgtcgaatgtgtacctactaaccctaaccgggtaaggctgataaatgccttcacgtagccaagtgtattgaggaatctcccggccgggggtaccgtctcgggctcccgttgcagttacttacgatggtctaaggtgcacctcccttcacatggttttccgcaacggaacccctcctgctcacctttaaagggggcaatttcaaatccatgatagctcattcatgtctgcgatcagtacctgttagacaatactaggcgcatttccccgatttaatagtatttatactgggactcagggccacttt

SEQ ID NO: 104 - DNA, 4599 bp, synthetic construct
  misc_feature 1..4599: note = Synthetic Nucleic Acid
  source 1..4599: mol_type = other DNA
cagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgagtactcctaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaaagcttattggacgtcgcttagcggtaccgccaccatgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttctaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcacagtgtacatcatgaccatggtctagagtagacgataaccacactgacgctaagacatcgttatttttttagacactttaccctctgttattatccccgccgggaatggctacccggatcatatacaccgacgtgccaagttcccgccctgtctccttatttccgctcggtgcaagattacagacgtccgaagtgtattagagtagacgcatcagtatcacttgtgtccttaaaccgacaggagaagtgctagtacgctgtgtaaaggatcctgaatataatataggacaccgtctattatatgtatataggaaacttatgtcgtatttgggcggaccaccaatccgattccaaaccccggttggaacgtgtcggatcgacactcgcccaaaatgaaatttaggctcttaaatgcaaaatctggttcgtaaattcagtcgggatagagacagttaacggttgcttgtctccatgacctacacatggtgcagggtgtcttggggataccagattgttatataacggtgatcaaacaagtagttccaaaagaaatatacagaagatccacagctaggccatatcctttaagagttagttatcacagcaacctcgctaactggcgtagagcgtaagccgcggttcgcaggtcataccactttatgattagtccttgtcgaatgtgtacctactaaccctaaccgggtaaggctgataaatgccttcacgtagccaagtgtattgaggaatctcccggccgggggtaccgtctcgggctcccgttgcagttacttacgatggtctaaggtgcacctcccttcacatggttttccgcaacggaacccctcctgctcacctttaaagggggcaatttcaaatccatgatagctcattcatgtctgcgatcagtacctgttagacaatactaggcgcatttccccgatttaatagtatttatactgggactcagggccactttgggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctagctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattacctaggttcctgcagg

SEQ ID NO: 105 - DNA, 182 bp, synthetic construct
  misc_feature 1..182: note = Synthetic Nucleic Acid
  source 1..182: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctag

SEQ ID NO: 106 - DNA, 178 bp, synthetic construct; identical to SEQ ID NO: 2
  misc_feature 1..178: note = Synthetic Nucleic Acid
  source 1..178: mol_type = other DNA

SEQ ID NO: 107 - DNA, 256 bp, synthetic construct
  misc_feature 1..256: note = Synthetic Nucleic Acid
  source 1..256: mol_type = other DNA
gcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggatatccgaggacagggccctgtctcctcagcttcaggcaccaccactgacctgggacagt

SEQ ID NO: 108 - DNA, 139 bp, synthetic construct
  misc_feature 1..139: note = Synthetic Nucleic Acid
  source 1..139: mol_type = other DNA
tgtttattgcagcttataatggttacaaataaagcaatagcatcacaaatttcacaaataaagcatttttttcactgcattctagttgtggtttgtccaaactcatcaatgtatcttatcatgtctggcgctagcagca

SEQ ID NO: 109 - DNA, 1906 bp, synthetic construct
  misc_feature 1..1906: note = Synthetic Nucleic Acid
  source 1..1906: mol_type = other DNA
cctcgcaggaggcctggccggcctccggattacttccggtgcaggcgtcagctgcatcttgctggccagctcggaggactgtccccgtaactcccgtccgctgtgtatcaacacagctctagccaaattgttgaccgaacgacggggttcgattgtttcattcctcacatccaacacttgagtagtacaatgccttgtctaagtgcgcttgggacccagtctattttgtttgtgtgcacaagacgatactcctcgatcacgcaccaagcgcaggctggccctaacgacgtggtattctcgtattggagagccccagattgcataaggcactcatcacctgcgtttatgacgctggacgcctgcgtatgttgcaggactcctatgatgtcgcttgcctgatgcgttgttatgtgattgaggtttagtacttggcaatatcattagatggaactaatacgcggtctaatcgttacaggtttccgacgattgagaagtatcacctgattctcgggagcgacttgtaacactactacacttattgatttggtactcctatgtgttgctatcttgactccgtaaatcgccgtgatacactctcttcgtttctcggcaatctatggctctaaactatacacaggcattctaagaataatctgacctgtccctgtagtactggtaggttactaccagtacggcgacaatgtgggagctacggcgtggtagaaccgggtacagacacagtccctgatcccgatagtaacactgtgcccgtagcagtgataatcattgcgcctcaacctgaggaagataagggaggcagtatatcgtgctattccctctattctcccacatgcgtatggcctgaggtcaatgttcaggtacatgccgtaatatgaggttttacgtagccgatcacgaaaaggttgctaaatacttctcgtcatatcccagctcattgttgggattatcgctaaacaggctaagttctaatgcccgtgggcatggcctcacgatctatggtgatacagacagcccgatttagcaatgcttcagagcacagtggtaaatattggttgatacgtagaaccatgatcactgctcaattcgtgatgaagttgttcaatatacctagaagccactcaaggattctctctgagtcactaatagaggtggtagagtgtttggaagccggttccacacagatttagagtgtcacctaattgccgtttatctaaggaattacgacaggagataccgaagtaattaggctattgcagagatacaggctaggtgaggtggagggtgtcttgcggtcttcgtccagccgccttgaaagcggggtttttttcaagcatcgctgcgtcattgcgtggtcgagacttatgcgccctacgaagttcgtctgaagtagtccaggaaagacctactttgcagttatcttcgcattcccacactcaccactacaactactcttccctcaatttcccggttagtttcgctaagctccgaccttgggttactgtgttgcatccgactcgctgcggctttctagtacgctgtactgtttcattcttctgtaggtctggttccgtaagtccgaatttccaggccgtggtctagtcctaattattttctgtcccggtagctatatttagccgagggtttgtccatttgcccggcgtagagcgccgcgtttgcgaacatttgcgcccgtaatacgtagggacaccgtcgggtaatggatggcaaaagccgaaaacggcgtcttccggcgcttggattcagcgctcttgagccataaaccgcgttgcttctttggttaattcgtattaatgatcctaagcgccagcttattcgttaagaggcactaggcgcgccgcggcatgcgatcgccagcatggctacg

SEQ ID NO: 110 - DNA, 7302 bp, synthetic construct
  misc_feature 1..7302: note = Synthetic Nucleic Acid
  source 1..7302: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggagatcggtacttcgcgaatgcgtcgagatggtaccaccggtactgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcttaagcctaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggatatccgaggacagggccctgtctcctcagcttcaggcaccaccactgacctgggacagtgaatcgcaagaattcacgcgtcaattgctcgaggccaccatggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttctgatgagatgcattcgaagcggccgcgagctcaagcttgcaattccgataacttgtttattgcagcttataatggttacaaataaagcaatagcatcacaaatttcacaaataaagcatttttttcactgcattctagttgtggtttgtccaaactcatcaatgtatcttatcatgtctggcgctagcagcacctcgcaggaggcctggccggcctccggattacttccggtgcaggcgtcagctgcatcttgctggccagctcggaggactgtccccgtaactcccgtccgctgtgtatcaacacagctctagccaaattgttgaccgaacgacggggttcgattgtttcattcctcacatccaacacttgagtagtacaatgccttgtctaagtgcgcttgggacccagtctattttgtttgtgtgcacaagacgatactcctcgatcacgcaccaagcgcaggctggccctaacgacgtggtattctcgtattggagagccccagattgcataaggcactcatcacctgcgtttatgacgctggacgcctgcgtatgttgcaggactcctatgatgtcgcttgcctgatgcgttgttatgtgattgaggtttagtacttggcaatatcattagatggaactaatacgcggtctaatcgttacaggtttccgacgattgagaagtatcacctgattctcgggagcgacttgtaacactactacacttattgatttggtactcctatgtgttgctatcttgactccgtaaatcgccgtgatacactctcttcgtttctcggcaatctatggctctaaactatacacaggcattctaagaataatctgacctgtccctgtagtactggtaggttactaccagtacggcgacaatgtgggagctacggcgtggtagaaccgggtacagacacagtccctgatcccgatagtaacactgtgcccgtagcagtgataatcattgcgcctcaacctgaggaagataagggaggcagtatatcgtgctattccctctattctcccacatgcgtatggcctgaggtcaatgttcaggtacatgccgtaatatgaggttttacgtagccgatcacgaaaaggttgctaaatacttctcgtcatatcccagctcattgttgggattatcgctaaacaggctaagttctaatgcccgtgggcatggcctcacgatctatggtgatacagacagcccgatttagcaatgcttcagagcacagtggtaaatattggttgatacgtagaaccatgatcactgctcaattcgtgatgaagttgttcaatatacctagaagccactcaaggattctctctgagtcactaatagaggtggtagagtgtttggaagccggttccacacagatttagagtgtcacctaattgccgtttatctaaggaattacgacaggagataccgaagtaattaggctattgcagagatacaggctaggtgaggtggagggtgtcttgcggtcttcgtccagccgccttgaaagcggggtttttttcaagcatcgctgcgtcattgcgtggtcgagacttatgcgccctacgaagttcgtctgaagtagtccaggaaagacctactttgcagttatcttcgcattcccacactcaccactacaactactcttccctcaatttcccggttagtttcgctaagctccgaccttgggttactgtgttgcatccgactcgctgcggctttctagtacgctgtactgtttcattcttctgtaggtctggttccgtaagtccgaatttccaggccgtggtctagtcctaattattttctgtcccggtagctatatttagccgagggtttgtccatttgcccggcgtagagcgccgcgtttgcgaacatttgcgcccgtaatacgtagggacaccgtcgggtaatggatggcaaaagccgaaaacggcgtcttccggcgcttggattcagcgctcttgagccataaaccgcgttgcttctttggttaattcgtattaatgatcctaagcgccagcttattcgttaagaggcactaggcgcgccgcggcatgcgatcgccagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagactagtgtcgacatcggatgccgggaccgacgagtgcagaggcgtgcaagcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 111 - DNA, 2256 bp, synthetic construct
  misc_feature 1..2256: note = Synthetic Nucleic Acid
  source 1..2256: mol_type = other DNA
atgctgcgggccaagaatcaactgttcctgctgtcccctcactacctgcggcaagtgaaagagagcagcggcagcagactgatccagcagagactgctgcatcagcagcagccactgcaccctgaatgggccgctctggctaagaagcagctcaagggcaagaaccccgaggacctgatctggcacacaccagagggcatcagcatcaagcccctgtactccaagcgggacacaatggatctgcccgaggaactgcctggcgtgaagccttttacaagaggcccctatcctaccatgtataccttcagaccctggaccatccggcagtacgccggcttttctaccgtggaagagagcaacaagttctacaaggacaacatcaaggccggccagcagggactgagcgtggcatttgatctggctacccacaggggctacgacagcgacaaccctagagtgcggggagatgttggaatggccggcgtggcaatcgacacagtggaagataccaagatcctgttcgacggcatccctctggaaaagatgagcgtgtccatgaccatgaacggcgctgtgatccccgtgctggctaactttattgtgaccggcgaggaacagggcgtgcccaaagaaaagctgaccggcaccatccagaacgacatcctgaaagagttcatggttcgaaacacctacatcttcccacctgagccgagcatgaagatcattgccgacatcttcgagtacaccgccaagcacatgcccaagttcaacagcatctccatcagcggctaccacatgcaagaggctggcgccgatgccatcctggaactggcttatacactggccgacggcctggaatactccagaacaggactgcaagccggcctgaccatcgatgagtttgcccctagactgagcttcttctggggcatcggcatgaacttctacatggaaatcgccaagatgagagccggcagacggctgtgggctcacctgatcgagaagatgttccagcctaagaacagcaagagcctgctcctgagagcccactgtcagacaagtggctggtccctgactgagcaggacccctacaacaacatcgtgcgcacagccatcgaagctatggccgccgtgtttggcggaacacagagcctgcacaccaacagctttgacgaggctctgggcctgcctaccgtgaagtctgccagaatcgcccggaacacccagatcatcatccaagaggaaagcggcatccccaaggtggcagatccttggggcggcagctacatgatggaatgcctgaccaacgacgtgtacgacgccgctctgaagctgatcaacgagatcgaagagatgggcggcatggctaaggctgtggccgagggaatccccaagctgagaatcgaggaatgcgccgccagacggcaggccagaattgatagcggaagcgaagtgatcgtgggcgtgaacaagtaccagctcgaaaaagaggacgccgtcgaggtcctggctatcgacaataccagcgtgcggaaccggcagattgagaagctgaagaagatcaagagcagccgcgatcaggccctggccgaaagatgtcttgctgccctgacagagtgtgccgccagcggcgacggaaatattctggctctggccgtggatgccagccgggctagatgtaccgtgggcgagattacagacgccctgaagaaggtgttcggcgagcacaaggccaacgacagaatggtgtctggcgcctacagacaagagtttggcgagagcaaagagatcaccagcgccatcaagcgggtccacaagttcatggaaagagaaggcaggcggcccagactgctggtggctaagatgggacaagacggccatgacagaggcgccaaagtgatcgccacaggctttgccgatctgggcttcgacgtggacatcggccctctgtttcagacccctagagaggtggcacagcaggccgttgatgccgatgttcacgctgtgggcgtgtctacactggctgccggacacaagacactggtgcccgaactgatcaaagagctgaacagcctgggcagacccgacatccttgtgatgtgtggcggagtgatcccaccgcaggactacgagttcctgtttgaagtgggcgtgtccaacgtgttcggccctggcacaagaatccctaaagccgccgtgcaggttctggacgacatcgagaagtgcctggaaaaaaagcagcagagcgtgggatcc

SEQ ID NO: 112 - DNA, 2253 bp, synthetic construct
  misc_feature 1..2253: note = Synthetic Nucleic Acid
  source 1..2253: mol_type = other DNA
ctgcgggccaagaatcaactgttcctgctgtcccctcactacctgcggcaagtgaaagagagcagcggcagcagactgatccagcagagactgctgcatcagcagcagccactgcaccctgaatgggccgctctggctaagaagcagctcaagggcaagaaccccgaggacctgatctggcacacaccagagggcatcagcatcaagcccctgtactccaagcgggacacaatggatctgcccgaggaactgcctggcgtgaagccttttacaagaggcccctatcctaccatgtataccttcagaccctggaccatccggcagtacgccggcttttctaccgtggaagagagcaacaagttctacaaggacaacatcaaggccggccagcagggactgagcgtggcatttgatctggctacccacaggggctacgacagcgacaaccctagagtgcggggagatgttggaatggccggcgtggcaatcgacacagtggaagataccaagatcctgttcgacggcatccctctggaaaagatgagcgtgtccatgaccatgaacggcgctgtgatccccgtgctggctaactttattgtgaccggcgaggaacagggcgtgcccaaagaaaagctgaccggcaccatccagaacgacatcctgaaagagttcatggttcgaaacacctacatcttcccacctgagccgagcatgaagatcattgccgacatcttcgagtacaccgccaagcacatgcccaagttcaacagcatctccatcagcggctaccacatgcaagaggctggcgccgatgccatcctggaactggcttatacactggccgacggcctggaatactccagaacaggactgcaagccggcctgaccatcgatgagtttgcccctagactgagcttcttctggggcatcggcatgaacttctacatggaaatcgccaagatgagagccggcagacggctgtgggctcacctgatcgagaagatgttccagcctaagaacagcaagagcctgctcctgagagcccactgtcagacaagtggctggtccctgactgagcaggacccctacaacaacatcgtgcgcacagccatcgaagctatggccgccgtgtttggcggaacacagagcctgcacaccaacagctttgacgaggctctgggcctgcctaccgtgaagtctgccagaatcgcccggaacacccagatcatcatccaagaggaaagcggcatccccaaggtggcagatccttggggcggcagctacatgatggaatgcctgaccaacgacgtgtacgacgccgctctgaagctgatcaacgagatcgaagagatgggcggcatggctaaggctgtggccgagggaatccccaagctgagaatcgaggaatgcgccgccagacggcaggccagaattgatagcggaagcgaagtgatcgtgggcgtgaacaagtaccagctcgaaaaagaggacgccgtcgaggtcctggctatcgacaataccagcgtgcggaaccggcagattgagaagctgaagaagatcaagagcagccgcgatcaggccctggccgaaagatgtcttgctgccctgacagagtgtgccgccagcggcgacggaaatattctggctctggccgtggatgccagccgggctagatgtaccgtgggcgagattacagacgccctgaagaaggtgttcggcgagcacaaggccaacgacagaatggtgtctggcgcctacagacaagagtttggcgagagcaaagagatcaccagcgccatcaagcgggtccacaagttcatggaaagagaaggcaggcggcccagactgctggtggctaagatgggacaagacggccatgacagaggcgccaaagtgatcgccacaggctttgccgatctgggcttcgacgtggacatcggccctctgtttcagacccctagagaggtggcacagcaggccgttgatgccgatgttcacgctgtgggcgtgtctacactggctgccggacacaagacactggtgcccgaactgatcaaagagctgaacagcctgggcagacccgacatccttgtgatgtgtggcggagtgatcccaccgcaggactacgagttcctgtttgaagtgggcgtgtccaacgtgttcggccctggcacaagaatccctaaagccgccgtgcaggttctggacgacatcgagaagtgcctggaaaaaaagcagcagagcgtgggatcc

SEQ ID NO: 113 - DNA, 2250 bp, synthetic construct; identical to SEQ ID NO: 19
  misc_feature 1..2250: note = Synthetic Nucleic Acid
  source 1..2250: mol_type = other DNA

SEQ ID NO: 114 - DNA, 2247 bp, synthetic construct; identical to SEQ ID NO: 20
  misc_feature 1..2247: note = Synthetic Nucleic Acid
  source 1..2247: mol_type = other DNA

SEQ ID NO: 115 - DNA, 2253 bp, synthetic construct
  misc_feature 1..2253: note = Synthetic Nucleic Acid
  source 1..2253: mol_type = other DNA
atgctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaa

SEQ ID NO: 116 - DNA, 2250 bp, synthetic construct
  misc_feature 1..2250: note = Synthetic Nucleic Acid
  source 1..2250: mol_type = other DNA
ctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaa

SEQ ID NO: 117 - DNA, 2253 bp, synthetic construct
  misc_feature 1..2253: note = Synthetic Nucleic Acid
  source 1..2253: mol_type = other DNA
atgttaagagctaagaatcagctttttttactttcacctcattacctgaggcaggtaaaagaatcatcaggctccaggctcatacagcaacgacttctacaccagcaacagccccttcacccagaatgggctgccctggctaaaaagcagctgaaaggcaaaaacccagaagacctaatatggcacaccccggaagggatctctataaaacccttgtattccaagagagatactatggacttacctgaagaacttccaggagtgaagccattcacacgtggaccatatcctaccatgtatacctttaggccctggaccatccgccagtatgctggttttagtactgtggaagaaagcaataagttctataaggacaacattaaggctggtcagcagggattatcagttgcctttgatctggcgacacatcgtggctatgattcagacaaccctcgagttcgtggtgatgttggaatggctggagttgctattgacactgtggaagataccaaaattctttttgatggaattcctttagaaaaaatgtcagtttccatgactatgaatggagcagttattccagttcttgcaaattttatagtaactggagaagaacaaggtgtacctaaagagaagcttactggtaccatccaaaatgatatactaaaggaatttatggttcgaaatacatacatttttcctccagaaccatccatgaaaattattgctgacatatttgaatatacagcaaagcacatgccaaaatttaattcaatttcaattagtggataccatatgcaggaagcaggggctgatgccattctggagctggcctatactttagcagatggattggagtactctagaactggactccaggctggcctgacaattgatgaatttgcaccaaggttgtctttcttctggggaattggaatgaatttctatatggaaatagcaaagatgagagctggtagaagactctgggctcacttaatagagaaaatgtttcagcctaaaaactcaaaatctcttcttctaagagcacactgtcagacatctggatggtcacttactgagcaggatccctacaataatattgtccgtactgcaatagaagcaatggcagcagtatttggagggactcagtctttgcacacaaattcttttgatgaagctttgggtttgccaactgtgaaaagtgctcgaattgccaggaacacacaaatcatcattcaagaagaatctgggattcccaaagtggctgatccttggggaggttcttacatgatggaatgtctcacaaatgatgtttatgatgctgctttaaagctcattaatgaaattgaagaaatgggtggaatggccaaagctgtagctgagggaatacctaaacttcgaattgaagaatgtgctgcccgaagacaagctagaatagattctggttctgaagtaattgttggagtaaataagtaccagttggaaaaagaagacgctgtagaagttctggcaattgataatacttcagtgcgaaacaggcagattgaaaaacttaagaagatcaaatccagcagggatcaagctttggctgaacgttgtcttgctgcactaaccgaatgtgctgctagcggagatggaaatatcctggctcttgcagtggatgcatctcgggcaagatgtacagtgggagaaatcacagatgccctgaaaaaggtatttggtgaacataaagcgaatgatcgaatggtgagtggagcatatcgccaggaatttggagaaagtaaagagataacatctgctatcaagagggttcataaattcatggaacgtgaaggtcgcagacctcgtcttcttgtagcaaaaatgggacaagatggccatgacagaggagcaaaagttattgctacaggatttgctgatcttggttttgatgtggacataggccctcttttccagactcctcgtgaagtggcccagcaggctgtggatgcggatgtgcatgctgtgggcataagcaccctcgctgctggtcataaaaccctagttcctgaactcatcaaagaacttaactcccttggacggccagatattcttgtcatgtgtggaggggtgataccacctcaggattatgaatttctgtttgaagttggtgtttccaatgtatttggtcctgggactcgaattccaaaggctgccgttcaggtgcttgatgatattgagaagtgtttggaaaagaagcagcaatctgtataa

SEQ ID NO: 118 - DNA, 2250 bp, synthetic construct
  misc_feature 1..2250: note = Synthetic Nucleic Acid
  source 1..2250: mol_type = other DNA
ttaagagctaagaatcagctttttttactttcacctcattacctgaggcaggtaaaagaatcatcaggctccaggctcatacagcaacgacttctacaccagcaacagccccttcacccagaatgggctgccctggctaaaaagcagctgaaaggcaaaaacccagaagacctaatatggcacaccccggaagggatctctataaaacccttgtattccaagagagatactatggacttacctgaagaacttccaggagtgaagccattcacacgtggaccatatcctaccatgtatacctttaggccctggaccatccgccagtatgctggttttagtactgtggaagaaagcaataagttctataaggacaacattaaggctggtcagcagggattatcagttgcctttgatctggcgacacatcgtggctatgattcagacaaccctcgagttcgtggtgatgttggaatggctggagttgctattgacactgtggaagataccaaaattctttttgatggaattcctttagaaaaaatgtcagtttccatgactatgaatggagcagttattccagttcttgcaaattttatagtaactggagaagaacaaggtgtacctaaagagaagcttactggtaccatccaaaatgatatactaaaggaatttatggttcgaaatacatacatttttcctccagaaccatccatgaaaattattgctgacatatttgaatatacagcaaagcacatgccaaaatttaattcaatttcaattagtggataccatatgcaggaagcaggggctgatgccattctggagctggcctatactttagcagatggattggagtactctagaactggactccaggctggcctgacaattgatgaatttgcaccaaggttgtctttcttctggggaattggaatgaatttctatatggaaatagcaaagatgagagctggtagaagactctgggctcacttaatagagaaaatgtttcagcctaaaaactcaaaatctcttcttctaagagcacactgtcagacatctggatggtcacttactgagcaggatccctacaataatattgtccgtactgcaatagaagcaatggcagcagtatttggagggactcagtctttgcacacaaattcttttgatgaagctttgggtttgccaactgtgaaaagtgctcgaattgccaggaacacacaaatcatcattcaagaagaatctgggattcccaaagtggctgatccttggggaggttcttacatgatggaatgtctcacaaatgatgtttatgatgctgctttaaagctcattaatgaaattgaagaaatgggtggaatggccaaagctgtagctgagggaatacctaaacttcgaattgaagaatgtgctgcccgaagacaagctagaatagattctggttctgaagtaattgttggagtaaataagtaccagttggaaaaagaagacgctgtagaagttctggcaattgataatacttcagtgcgaaacaggcagattgaaaaacttaagaagatcaaatccagcagggatcaagctttggctgaacgttgtcttgctgcactaaccgaatgtgctgctagcggagatggaaatatcctggctcttgcagtggatgcatctcgggcaagatgtacagtgggagaaatcacagatgccctgaaaaaggtatttggtgaacataaagcgaatgatcgaatggtgagtggagcatatcgccaggaatttggagaaagtaaagagataacatctgctatcaagagggttcataaattcatggaacgtgaaggtcgcagacctcgtcttcttgtagcaaaaatgggacaagatggccatgacagaggagcaaaagttattgctacaggatttgctgatcttggttttgatgtggacataggccctcttttccagactcctcgtgaagtggcccagcaggctgtggatgcggatgtgcatgctgtgggcataagcaccctcgctgctggtcataaaaccctagttcctgaactcatcaaagaacttaactcccttggacggccagatattcttgtcatgtgtggaggggtgataccacctcaggattatgaatttctgtttgaagttggtgtttccaatgtatttggtcctgggactcgaattccaaaggctgccgttcaggtgcttgatgatattgagaagtgtttggaaaagaagcagcaatctgtataa

SEQ ID NO: 119 - DNA, 2250 bp, synthetic construct
  misc_feature 1..2250: note = Synthetic Nucleic Acid
  source 1..2250: mol_type = other DNA
atgctgcgggccaagaaccagctgttcctgctgagccctcactacctgcggcaggtgaaggagagcagcggcagccggctgatccagcagcggctgctgcaccagcagcagcccctgcaccccgagtgggccgccctggccaagaagcagctgaagggcaagaaccccgaggacctgatctggcacacgcccgagggcatcagcatcaagcccctgtacagcaagcgggacaccatggacctgcccgaggagctgcccggcgtgaagcccttcacccggggcccctaccccaccatgtacaccttccggccctggaccatccggcagtacgccggcttcagcaccgtggaggagagcaacaagttctacaaggacaacatcaaggccggccagcagggcctgagcgtggccttcgacctggccacccaccggggctacgacagcgacaacccacgggtgcggggcgacgtgggcatggccggcgtggccatcgacaccgtggaggacaccaagatcctgttcgacggcatccctctggagaagatgagcgtgagcatgaccatgaacggcgccgtgatccccgtgctggccaacttcatcgtgaccggcgaggagcagggcgtgcccaaggagaagctgaccggcaccatccagaacgacatcctgaaggagttcatggtgcggaacacctacatcttccctcccgagcccagcatgaagatcatcgccgacatcttcgagtacaccgccaagcacatgcccaagttcaacagcatcagcatcagcggctaccacatgcaggaggccggcgccgacgccatcctggagctggcctacaccctggccgacggcctggagtacagccggaccggcctgcaggccggcctgaccatcgacgagttcgcgccccggctgagcttcttctggggcatcggcatgaacttctacatggagatcgccaagatgcgggccggccggcggctgtgggcccacctgatcgagaagatgttccagcccaagaacagcaagagcctgctgctgcgggcccactgccagaccagcggctggagcctgaccgagcaggacccctacaacaacatcgtgcggaccgccatcgaggccatggccgccgtgttcggcggcacccagagcctgcacaccaacagcttcgacgaggccctgggcctgcccaccgtgaagagcgcccggatcgcccggaacacccagatcatcatccaggaggagagcggcatccccaaggtggccgacccctggggcggcagctacatgatggagtgcctgaccaacgacgtgtacgacgccgccctgaagctgatcaacgagatcgaggagatgggcggcatggccaaggccgtggccgagggcatccccaagctgcggatcgaggagtgcgccgcccggcggcaggcccggatcgacagcggcagcgaggtgatcgtgggcgtgaacaagtaccagctggagaaggaggacgccgtggaggtgctggccatcgacaacaccagcgtgcggaaccggcagatcgagaagctgaagaagatcaagagcagccgggaccaggccctggccgagcggtgcctggccgccctgaccgagtgcgccgccagcggcgacggcaacatcctggccctggccgtggacgccagccgggcccggtgcaccgtgggcgagatcaccgacgccctgaagaaggtgttcggcgagcacaaggccaacgaccggatggtgagcggcgcctaccggcaggagttcggcgagagcaaggagatcaccagcgccatcaagcgggtgcacaagttcatggagcgggagggccggcggccccggctgctggtggccaagatgggccaggacggccacgaccggggcgccaaggtgatcgccaccggcttcgccgacctgggcttcgacgtggacatcggcccactgttccagacgccccgggaggtggcccagcaggccgtggacgccgacgtgcacgccgtgggcgtgagcaccctggccgccggccacaagaccctggtgcccgagctgatcaaggagctgaacagcctgggccggcccgacatcctggtgatgtgcggcggcgtgatcccgccccaggactacgagttcctgttcgaggtgggcgtgagcaacgtgttcggccccggcacccggatccccaaggccgccgtgcaggtgctggacgacatcgagaagtgcctggagaagaagcagcagagcgtg

SEQ ID NO: 120 - DNA, 2247 bp, synthetic construct
  misc_feature 1..2247: note = Synthetic Nucleic Acid
  source 1..2247: mol_type = other DNA
ctgcgggccaagaaccagctgttcctgctgagccctcactacctgcggcaggtgaaggagagcagcggcagccggctgatccagcagcggctgctgcaccagcagcagcccctgcaccccgagtgggccgccctggccaagaagcagctgaagggcaagaaccccgaggacctgatctggcacacgcccgagggcatcagcatcaagcccctgtacagcaagcgggacaccatggacctgcccgaggagctgcccggcgtgaagcccttcacccggggcccctaccccaccatgtacaccttccggccctggaccatccggcagtacgccggcttcagcaccgtggaggagagcaacaagttctacaaggacaacatcaaggccggccagcagggcctgagcgtggccttcgacctggccacccaccggggctacgacagcgacaacccacgggtgcggggcgacgtgggcatggccggcgtggccatcgacaccgtggaggacaccaagatcctgttcgacggcatccctctggagaagatgagcgtgagcatgaccatgaacggcgccgtgatccccgtgctggccaacttcatcgtgaccggcgaggagcagggcgtgcccaaggagaagctgaccggcaccatccagaacgacatcctgaaggagttcatggtgcggaacacctacatcttccctcccgagcccagcatgaagatcatcgccgacatcttcgagtacaccgccaagcacatgcccaagttcaacagcatcagcatcagcggctaccacatgcaggaggccggcgccgacgccatcctggagctggcctacaccctggccgacggcctggagtacagccggaccggcctgcaggccggcctgaccatcgacgagttcgcgccccggctgagcttcttctggggcatcggcatgaacttctacatggagatcgccaagatgcgggccggccggcggctgtgggcccacctgatcgagaagatgttccagcccaagaacagcaagagcctgctgctgcgggcccactgccagaccagcggctggagcctgaccgagcaggacccctacaacaacatcgtgcggaccgccatcgaggccatggccgccgtgttcggcggcacccagagcctgcacaccaacagcttcgacgaggccctgggcctgcccaccgtgaagagcgcccggatcgcccggaacacccagatcatcatccaggaggagagcggcatccccaaggtggccgacccctggggcggcagctacatgatggagtgcctgaccaacgacgtgtacgacgccgccctgaagctgatcaacgagatcgaggagatgggcggcatggccaaggccgtggccgagggcatccccaagctgcggatcgaggagtgcgccgcccggcggcaggcccggatcgacagcggcagcgaggtgatcgtgggcgtgaacaagtaccagctggagaaggaggacgccgtggaggtgctggccatcgacaacaccagcgtgcggaaccggcagatcgagaagctgaagaagatcaagagcagccgggaccaggccctggccgagcggtgcctggccgccctgaccgagtgcgccgccagcggcgacggcaacatcctggccctggccgtggacgccagccgggcccggtgcaccgtgggcgagatcaccgacgccctgaagaaggtgttcggcgagcacaaggccaacgaccggatggtgagcggcgcctaccggcaggagttcggcgagagcaaggagatcaccagcgccatcaagcgggtgcacaagttcatggagcgggagggccggcggccccggctgctggtggccaagatgggccaggacggccacgaccggggcgccaaggtgatcgccaccggcttcgccgacctgggcttcgacgtggacatcggcccactgttccagacgccccgggaggtggcccagcaggccgtggacgccgacgtgcacgccgtgggcgtgagcaccctggccgccggccacaagaccctggtgcccgagctgatcaaggagctgaacagcctgggccggcccgacatcctggtgatgtgcggcggcgtgatcccgccccaggactacgagttcctgttcgaggtgggcgtgagcaacgtgttcggccccggcacccggatccccaaggccgccgtgcaggtgctggacgacatcgagaagtgcctggagaagaagcagcagagcgtg

SEQ ID NO: 121 - protein, 750 aa, synthetic construct [sequence omitted]

SEQ ID NO: 122 - protein, 749 aa, synthetic construct
  REGION 1..749: note = Synthetic Nucleic Acid
LRAKNQLFLLSPHYLRQVKESSGSRLIQQRLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGVSTLAAGHKTLVPELIKELNSLGRPDILVMCGGVIPPQDYEFLFEVGVSNVFGPGTRIPKAAVQVLDDIEKCLEKKQQSV

SEQ ID NO: 123 - protein, 354 aa, synthetic construct
  REGION 1..354: note = Synthetic Sequence
MLSNLRILLNNAALRKGHTSVVRHFWCGKPVQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 124 - protein, 353 aa, synthetic construct
  REGION 1..353: note = Synthetic Sequence
LSNLRILLNNAALRKGHTSVVRHFWCGKPVQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 125 - DNA, 175 bp, synthetic construct
  misc_feature 1..175: note = Synthetic Construct
  source 1..175: mol_type = other DNA
tgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaactttta

SEQ ID NO: 126 - DNA, 255 bp, synthetic construct
  misc_feature 1..255: note = Synthetic Sequence
  source 1..255: mol_type = other DNA
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgca

SEQ ID NO: 127 - DNA, 141 bp, synthetic construct
  misc_feature 1..141: note = Synthetic Sequence
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 128 - DNA, 122 bp, synthetic construct; identical to SEQ ID NO: 97
  misc_feature 1..122: note = Synthetic Sequence
  source 1..122: mol_type = other DNA

SEQ ID NO: 129 - DNA, 1445 bp, synthetic construct
  misc_feature 1..1445: note = Synthetic Sequence
  source 1..1445: mol_type = other DNA
tttcctggactacttcagacgaacttcgtagggcgcataagtctcgaccacgcaatgacgcagcgatgcttgaaaaaaacaccgctttcaaggcggctggacgaagaccgcaagacaccctccacctcacctagcctgtatctctgcaatagcctaattacttcggaatctcctgtcgtaattccttagataaacggcaattaggtgacactctaaatctgtgtggaaccggcttccaaacactctaccacctctattagtgacacagagagaatccttgagtggcttctaggtatattgaacaacttcatcacgaattgagcagtgatcatggttctacgtatcaaccaatattaaccactgtgctctgtagcattgctaaatcgggctgtctgtttcaccatagatcgtgaggccatgcccacgggcattagaacttagcctgtttagcgataatcccaacaatgagctgggatatgacgagaagtatttagcaaccttttcgtgatcggctacgtaaaacctcatattacggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtactggtagtaacctaccagtactacagggacaggtcagattattcttagaatgcctgtgtatagtttagagccatagattgccgagaaacgaagagagtgtatcacggcgatttacggagtcaagatagcaacacataggagtaccaaatcaataagtgtagtagagttacaagtcgctcccgagaatcaggtgatacttctcaatcgtcggaaacctgtaacgattagaccgcgtattagttccatctaatgatattgccaagtactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgc

SEQ ID NO: 130 - DNA, 1445 bp, synthetic construct
  misc_feature 1..1445: note = Synthetic Sequence
  source 1..1445: mol_type = other DNA
tttcctggactacttcagacgaacttcgtagggcgcataagtctcgaccacgcaatgacgcagcgatgcttgaaaaaaaccccgctttcaaggcggctggacgaagaccgcaagacaccctccacctcacctagcctgtatctctgcaatagcctaattacttcggaatctcctgtcgtaattccttagataaacggcaattaggtgacactctaaatctgtgtggaaccggcttccaaacactctaccacctctattagtgacacagagagaatccttgagtggcttctaggtatattgaacaacttcatcacgaattgagcagtgatcatggttctacgtatcaaccaatattaaccactgtgctctgtagcattgctaaatcgggctgtctgtttcaccatagatcgtgaggccatgcccacgggcattagaacttagcctgtttagcgataatcccaacaatgagctgggatatgacgagaagtatttagcaaccttttcgtgatcggctacgtaaaacctcatattacggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtactggtagtaacctaccagtactacagggacaggtcagattattcttagaatgcctgtgtatagtttagagccatagattgccgagaaacgaagagagtgtatcacggcgatttacggagtcaagatagcaacacataggagtaccaaatcaataagtgtagtagagttacaagtcgctcccgagaatcaggtgatacttctcaatcgtcggaaacctgtaacgattagaccgcgtattagttccatctaatgatattgccaagtactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgc

SEQ ID NO: 131 - DNA, 927 bp, synthetic construct
  misc_feature 1..927: note = Synthetic Sequence
  source 1..927: mol_type = other DNA
tttcctgtcgacggaggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtactggtagtaacctaccagtactacagggacaggtcagattattcttagaatgcctgtgtatagtttagagccatagattgccgagaaacgaagagagtgtatcacggcgatttacggagtcaagatagcaacacataggagtaccaaatcaataagtgtagtagagttacaagtcgctcccgagaatcaggtgatacttctcaatcgtcggaaacctgtaacgattagaccgcgtattagttccatctaatgatattgccaagtactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgc

SEQ ID NO: 132 - DNA, 435 bp, synthetic construct
  misc_feature 1..435: note = Synthetic Sequence
  source 1..435: mol_type = other DNA
aaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 133 - DNA, 717 bp, synthetic construct
  misc_feature 1..717: note = Synthetic Sequence
  source 1..717: mol_type = other DNA
atggtttccaagggcgaagaactgtttaccggcgtggtgcccatcctggtggaactggatggcgacgttaacggacacaagttcagcgtcagcggagaaggcgaaggcgacgccacatacggaaagctgacactgaagtttatctgcaccaccggcaagctgcccgtgccttggcctacactggtcaccacactgacatacggcgtgcagtgcttcagcagataccccgaccacatgaagcagcacgatttcttcaagagcgccatgcctgagggctacgtgcaagagcggaccatcttcttcaaggacgacgggaactacaagaccagagccgaagtgaagttcgagggcgacaccctcgtgaaccggatcgagctgaagggcatcgacttcaaagaggacggaaacatcctgggccacaaacttgagtacaactacaacagccacaacgtctacatcatggccgacaagcagaaaaacggcatcaaagtgaacttcaagatccggcacaacatcgaggacggctctgtgcagctggctgaccactaccagcagaacacacccatcggagatggccctgtgctgctgcccgataaccactacctgagcacacagagcgccctgagcaaggaccccaacgagaagagggatcacatggtgctgctggaatttgtgaccgctgccggcatcaccctcggcatggatgaactgtacaag

SEQ ID NO: 134 - DNA, 628 bp, synthetic construct
  misc_feature 1..628: note = Synthetic Sequence
  source 1..628: mol_type = other DNA
atggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgatctagagatctcatatgcctttaattaaacactagttctatagtgtcacctaaattccctttagtgagggttaatggccgtaggccgccagaattgggtccagacatga

SEQ ID NO: 135 - DNA, 63 bp, synthetic construct
  misc_feature 1..63: note = Synthetic Sequence
  source 1..63: mol_type = other DNA
ggatccggagaaggacggggaagcctgcttacatgcggagatgtggaggagaatcctggtccc

SEQ ID NO: 136 - DNA, 122 bp, synthetic construct
  misc_feature 1..122: note = Synthetic Sequence
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 137 - DNA, 1906 bp, synthetic construct
  misc_feature 1..1906: note = Synthetic Sequence
  source 1..1906: mol_type = other DNA
cctcgcaggaggcctggccggcctccggattacttccggtgcaggcgtcagctgcatcttgctggccagctcggaggactgtccccgtaactcccgtccgctgtgtatcaacacagctctagccaaattgttgaccgaacgacggggttcgattgtatcattcctcacatccaacacttgagtagtacaatgccttgtctaagtgcgcttgggacccagtctattttgtttgtgtgcacaagacgatactcctcgatcacgtaccaagcgcaggctggccctaacgacgtggtattctcgtattggtgtgccccagattgcataaggcactcatcacctgcgtttatgacgctggacgcctgcgtatgttgcaggactcctatgatgtcgctagcctgatgcgttgttatgtgattgaggtttagtacttggcaatatcattagatggaactaatacgcggtctaatcgttacaggtttccgacgattgagaagtatcacctgattctcgggagcgacttgtaactctactacacttattgatttggtactcctatgtgttgctatcttgactccgtaaatcgccgtgatacactctcttcgtttctcggcaatctatggctctaaactatacacaggcattctaagaataatctgacctgtccctgtagtactggtaggttactaccagtacggcgacaatgtgggagctacggcgtggtagaaccgggtacagacacagtccctgatcccgatagtaacactgtgcccgtagcagtgataatcattgcgcctcaacctgaggaagataagggaggcagtatatcgtgctattccctctattctcccacatgcgtatggcctgaggtcaatgatcaggtacatgccgtaatatgaggttttacgtagccgatcacgaaaaggttgctaaatacttctcgtcatatcccagctcattgttgggattatcgctaaacaggctaagttctaatgcccgtgggcatggcctcacgatctatggtgaaacagacagcccgatttagcaatgctacagagcacagtggttaatattggttgatacgtagaaccatgatcactgctcaattcgtgatgaagttgttcaatatacctagaagccactcaaggattctctctgtgtcactaatagaggtggtagagtgtttggaagccggttccacacagatttagagtgtcacctaattgccgtttatctaaggaattacgacaggagattccgaagtaattaggctattgcagagatacaggctaggtgaggtggagggtgtcttgcggtcttcgtccagccgccttgaaagcggggtttttttcaagcatcgctgcgtcattgcgtggtcgagacttatgcgccctacgaagttcgtctgaagtagtccaggaaagacctactttgcagttatcttcgcattcccacactcaccactacaactactcttccctcaatttcccggttagtttcgctaagctccgaccttgggttactgtgttgcatccgactcgctgcggctttctagtacgctgtactgtttcattcttctgtaggtctggttccgtaagtccgaatttccaggccgtggtctagtcctaattattttctgtcccggtagctatatttagccgagggtttgtccatttgcccggcgtagagcgccgcgtttgcgaacatttgcgcccgtaatacgtagggacaccgtcgggtaatggatggcaaaagccgaaaacggcgtcttccggcgcttggattcagcgctcttgagccataaaccgcgttgcttctttggttaattcgtattaatgatcctaagcgccagcttattcgttaagaggcactaggcgcgccgcggcatgcgatcgccagcatggctacg

SEQ ID NO: 138 - DNA, 4596 bp, synthetic construct
  misc_feature 1..4596: note = Synthetic Sequence
  source 1..4596: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgagtactcctaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaaagcttattggacgtcgcttagcggtaccgccaccatgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttctaatgaagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctttcctggactacttcagacgaacttcgtagggcgcataagtctcgaccacgcaatgacgcagcgatgcttgaaaaaaacaccgctttcaaggcggctggacgaagaccgcaagacaccctccacctcacctagcctgtatctctgcaatagcctaattacttcggaatctcctgtcgtaattccttagataaacggcaattaggtgacactctaaatctgtgtggaaccggcttccaaacactctaccacctctattagtgacacagagagaatccttgagtggcttctaggtatattgaacaacttcatcacgaattgagcagtgatcatggttctacgtatcaaccaatattaaccactgtgctctgtagcattgctaaatcgggctgtctgtttcaccatagatcgtgaggccatgcccacgggcattagaacttagcctgtttagcgataatcccaacaatgagctgggatatgacgagaagtatttagcaaccttttcgtgatcggctacgtaaaacctcatattacggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtactggtagtaacctaccagtactacagggacaggtcagattattcttagaatgcctgtgtatagtttagagccatagattgccgagaaacgaagagagtgtatcacggcgatttacggagtcaagatagcaacacataggagtaccaaatcaataagtgtagtagagttacaagtcgctcccgagaatcaggtgatacttctcaatcgtcggaaacctgtaacgattagaccgcgtattagttccatctaatgatattgccaagtactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 139 - DNA, 4790 bp, synthetic construct
  misc_feature 1..4790: note = Synthetic Sequence
  source 1..4790: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgagtactcctaggcgcgcccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcaagcttattggacgtcgcttagcggtaccgccaccatgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttctaatgaagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctttcctggactacttcagacgaacttcgtagggcgcataagtctcgaccacgcaatgacgcagcgatgcttgaaaaaaaccccgctttcaaggcggctggacgaagaccgcaagacaccctccacctcacctagcctgtatctctgcaatagcctaattacttcggaatctcctgtcgtaattccttagataaacggcaattaggtgacactctaaatctgtgtggaaccggcttccaaacactctaccacctctattagtgacacagagagaatccttgagtggcttctaggtatattgaacaacttcatcacgaattgagcagtgatcatggttctacgtatcaaccaatattaaccactgtgctctgtagcattgctaaatcgggctgtctgtttcaccatagatcgtgaggccatgcccacgggcattagaacttagcctgtttagcgataatcccaacaatgagctgggatatgacgagaagtatttagcaaccttttcgtgatcggctacgtaaaacctcatattacggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtactggtagtaacctaccagtactacagggacaggtcagattattcttagaatgcctgtgtatagtttagagccatagattgccgagaaacgaagagagtgtatcacggcgatttacggagtcaagatagcaacacataggagtaccaaatcaataagtgtagtagagttacaagtcgctcccgagaatcaggtgatacttctcaatcgtcggaaacctgtaacgattagaccgcgtattagttccatctaatgatattgccaagtactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 140 - DNA, 4700 bp, synthetic construct
  misc_feature 1..4700: note = Synthetic Sequence
  source 1..4700: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgagtactcctaggagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccagtggcaaagcttattggacgtcgcttagcggtaccgccaccatgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttctaatgaagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctttcctgtcgacggaggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtactggtagtaacctaccagtactacagggacaggtcagattattcttagaatgcctgtgtatagtttagagccatagattgccgagaaacgaagagagtgtatcacggcgatttacggagtcaagatagcaacacataggagtaccaaatcaataagtgtagtagagttacaagtcgctcccgagaatcaggtgatacttctcaatcgtcggaaacctgtaacgattagaccgcgtattagttccatctaatgatattgccaagtactaaacctcaatcacataacaacgcatcaggctagcgacatcataggagtcctgcaacatacgcaggcgtccagcgtcataaacgcaggtgatgagtgccttatgcaatctggggcacaccaatacgagaataccacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 141 - DNA, 4531 bp, synthetic construct
  misc_feature 1..4531: note = Synthetic Sequence
  source 1..4531: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgagtactcctaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaaagcttattggacgtcgcttagcggtaccgccaccatgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttcggatccggcgaaggcagaggctcactgcttacttgtggcgacgtggaggagaaccccggacctatggtttccaagggcgaagaactgtttaccggcgtggtgcccatcctggtggaactggatggcgacgttaacggacacaagttcagcgtcagcggagaaggcgaaggcgacgccacatacggaaagctgacactgaagtttatctgcaccaccggcaagctgcccgtgccttggcctacactggtcaccacactgacatacggcgtgcagtgcttcagcagataccccgaccacatgaagcagcacgatttcttcaagagcgccatgcctgagggctacgtgcaagagcggaccatcttcttcaaggacgacgggaactacaagaccagagccgaagtgaagttcgagggcgacaccctcgtgaaccggatcgagctgaagggcatcgacttcaaagaggacggaaacatcctgggccacaaacttgagtacaactacaacagccacaacgtctacatcatggccgacaagcagaaaaacggcatcaaagtgaacttcaagatccggcacaacatcgaggacggctctgtgcagctggctgaccactaccagcagaacacacccatcggagatggccctgtgctgctgcccgataaccactacctgagcacacagagcgccctgagcaaggaccccaacgagaagagggatcacatggtgctgctggaatttgtgaccgctgccggcatcaccctcggcatggatgaactgtacaagggctccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgatctagagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 142 - DNA, 4725 bp, synthetic construct
  misc_feature 1..4725: note = Synthetic Sequence
  source 1..4725: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgagtactcctaggcgcgcccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcaagcttattggacgtcgcttagcggtaccgccaccatgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttcggatccggcgaaggcagaggctcactgcttacttgtggcgacgtggaggagaaccccggacctatggtttccaagggcgaagaactgtttaccggcgtggtgcccatcctggtggaactggatggcgacgttaacggacacaagttcagcgtcagcggagaaggcgaaggcgacgccacatacggaaagctgacactgaagtttatctgcaccaccggcaagctgcccgtgccttggcctacactggtcaccacactgacatacggcgtgcagtgcttcagcagataccccgaccacatgaagcagcacgatttcttcaagagcgccatgcctgagggctacgtgcaagagcggaccatcttcttcaaggacgacgggaactacaagaccagagccgaagtgaagttcgagggcgacaccctcgtgaaccggatcgagctgaagggcatcgacttcaaagaggacggaaacatcctgggccacaaacttgagtacaactacaacagccacaacgtctacatcatggccgacaagcagaaaaacggcatcaaagtgaacttcaagatccggcacaacatcgaggacggctctgtgcagctggctgaccactaccagcagaacacacccatcggagatggccctgtgctgctgcccgataaccactacctgagcacacagagcgccctgagcaaggaccccaacgagaagagggatcacatggtgctgctggaatttgtgaccgctgccggcatcaccctcggcatggatgaactgtacaagggctccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgatctagagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 143 - DNA, 4363 bp, synthetic construct
  misc_feature 1..4363: note = Synthetic Sequence
  source 1..4363: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgagtactcctaggagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgaagcttattggacgtcgcttagcggtaccgccaccatgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttcggatccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgatctagagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 144 - DNA, 4651 bp, synthetic construct
  misc_feature 1..4651: note = Synthetic Sequence
  source 1..4651: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatggcgcgcctccagatatcatacactcgagcaactttgtatagaaaagttggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaagaattcacgcgtcaattgctcgaggccaccatggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttctgatgagatgcattcgaagcggccgcgagctcaagcttgcaattccgataacttgtttattgcagcttataatggttacaaataaagcaatagcatcacaaatttcacaaataaagcatttttttcactgcattctagttgtggtttgtccaaactcatcaatgtatcttatcatgtctggcgctagcagcacaagtttgtacaaaaaagcaggctcctcgcaggaggcctggccggcctccggattacttccggtgcaggcgtcagctgcatcttgctggccagctcggaggactgtccccgtaactcccgtccgctgtgtatcaacacagctctagccaaattgttgaccgaacgacggggttcgattgtatcattcctcacatccaacacttgagtagtacaatgccttgtctaagtgcgcttgggacccagtctattttgtttgtgtgcacaagacgatactcctcgatcacgtaccaagcgcaggctggccctaacgacgtggtattctcgtattggtgtgccccagattgcataaggcactcatcacctgcgtttatgacgctggacgcctgcgtatgttgcaggactcctatgatgtcgctagcctgatgcgttgttatgtgattgaggtttagtacttggcaatatcattagatggaactaatacgcggtctaatcgttacaggtttccgacgattgagaagtatcacctgattctcgggagcgacttgtaactctactacacttattgatttggtactcctatgtgttgctatcttgactccgtaaatcgccgtgatacactctcttcgtttctcggcaatctatggctctaaactatacacaggcattctaagaataatctgacctgtccctgtagtactggtaggttactaccagtacggcgacaatgtgggagctacggcgtggtagaaccgggtacagacacagtccctgatcccgatagtaacactgtgcccgtagcagtgataatcattgcgcctcaacctgaggaagataagggaggcagtatatcgtgctattccctctattctcccacatgcgtatggcctgaggtcaatgatcaggtacatgccgtaatatgaggttttacgtagccgatcacgaaaaggttgctaaatacttctcgtcatatcccagctcattgttgggattatcgctaaacaggctaagttctaatgcccgtgggcatggcctcacgatctatggtgaaacagacagcccgatttagcaatgctacagagcacagtggttaatattggttgatacgtagaaccatgatcactgctcaattcgtgatgaagttgttcaatatacctagaagccactcaaggattctctctgtgtcactaatagaggtggtagagtgtttggaagccggttccacacagatttagagtgtcacctaattgccgtttatctaaggaattacgacaggagattccgaagtaattaggctattgcagagatacaggctaggtgaggtggagggtgtcttgcggtcttcgtccagccgccttgaaagcggggtttttttcaagcatcgctgcgtcattgcgtggtcgagacttatgcgccctacgaagttcgtctgaagtagtccaggaaagacctactttgcagttatcttcgcattcccacactcaccactacaactactcttccctcaatttcccggttagtttcgctaagctccgaccttgggttactgtgttgcatccgactcgctgcggctttctagtacgctgtactgtttcattcttctgtaggtctggttccgtaagtccgaatttccaggccgtggtctagtcctaattattttctgtcccggtagctatatttagccgagggtttgtccatttgcccggcgtagagcgccgcgtttgcgaacatttgcgcccgtaatacgtagggacaccgtcgggtaatggatggcaaaagccgaaaacggcgtcttccggcgcttggattcagcgctcttgagccataaaccgcgttgcttctttggttaattcgtattaatgatcctaagcgccagcttattcgttaagaggcactaggcgcgccgcggcatgcgatcgccagcatggctacgacccagctttcttgtacaaagtggtgatggccggccgcttcgagttaattaatccaaccggttaccgcctaggatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 145 - DNA, 689 bp, synthetic construct
  misc_feature 1..689: note = Synthetic Sequence
  source 1..689: mol_type = other DNA
cgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgccacc

SEQ ID NO: 146 - DNA, 336 bp, synthetic construct
  misc_feature 1..336: note = Synthetic Sequence
  source 1..336: mol_type = other DNA
gtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacag